(19) United States
(12) Patent Application Publication
(10) Pub. No.: US 2026/0347765 A1
(43) Pub. Date: Nov. 5, 2026

(54) BOVINE TROPHOBLAST STEM CELLS AND USES THEREOF

(71) Applicant: Board of Supervisors of Louisiana State University and Agricultural and Mechanical College, Baton Rouge, LA (US)

(72) Inventor: Zongliang Carl Jiang, Baton Rouge, LA (US)

(21) Appl. No.: 19/099,806

(22) PCT Filed: Aug. 2, 2023
(86) PCT No.: PCT/US2023/029334

§ 371 Date: Jan. 30, 2025

Related U.S. Application Data
(60) Provisional application No. 63/370,192, filed on Aug. 2, 2022.
(60) Provisional application No. 63/413,789, filed on Oct. 6, 2022.

(51) Int. Cl. C12N 5/073; C12N 5/0735; G01N 33/50

(52) U.S. Cl. CPC C12N 5/0605; C12N 5/0606; G01N 33/5073; C12N 2501/235; C12N 2501/50; C12N 2501/727; C12N 2501/734; C12N 2502/1323; C12N 2503/02; C12N 2533/52; C12N 2533/54

(57) ABSTRACT

The present invention is directed to bovine trophoblast stem cells and uses thereof.

This application is an International Application which claims priority from U.S. Provisional Patent Application No. 63/370,192 filed on Aug. 2, 2022, and U.S. Provisional Patent Application No. 63/413,789 filed on Oct. 6, 2022, respectively, the contents of which are incorporated by reference herein in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under Grant No. 2019-67016-29863, awarded by the USDA National Institute of Food and Agriculture, and Grant No. R01HD102533, awarded by the NIH Eunice Kennedy Shriver National Institute of Child Health and Human Development (NICHD). The government has certain rights in the invention.

### FIELD OF THE INVENTION

This invention is directed to bovine trophoblast stem cells and uses thereof.

### SUMMARY OF THE INVENTION

An aspect of the invention is directed to bovine trophoblast stem cells and uses thereof.

In embodiments, aspects of the invention are drawn towards a method of culturing, expanding or growing a population of cells derived from a mammalian blastocyst. For example, the method comprises culturing the cells derived from a mammalian blastocyst for a period of time in a culture medium, wherein the culture medium comprises human leukemia inhibitory factor (hLIF), an inhibitor of glycogen synthase kinase-3 (GSK-3), an antagonist of muscarinic M2 and histamine H1 receptors, an inhibitor of matrix metalloproteinase (MMP), or any combination thereof.

In embodiments, the cultured cells remain in an undifferentiated state.

In embodiments, the cultured cells are capable of differentiation.

In embodiments, the method further comprises placing a mammalian blastocyst in a vessel seeded with fibroblast cells and adding the culture medium, thereby providing a population of cells derived from a mammalian blastocyst. For example, the fibroblast cells comprise mouse embryonic fibroblast cells. In embodiments, the trophoblast stem cells are cultured without fibroblast feeder cells.

In embodiments, the population of cells comprises trophoblast stem cells, trophoblast stem-like cells, or derivatives thereof.

In embodiments, the mammal is a bovine.

In embodiments, the GSK-3 inhibitor comprises CHIR99021. For example, the amount of the glycogen synthase kinase-3 inhibitor is about 3 μM.

In embodiments, the antagonist of muscarinic M2 and histamine H1 receptors comprises dimethindene maleate (DiM). For example, the amount of the muscarinic M2 and histamine H1 receptor antagonist is about 2 μM.

In embodiments, the inhibitor of matrix metalloproteinase (MMP) comprises minocycline hydrochloride (MiH). For example, the amount of the matrix metalloproteinase inhibitor is about 2 μM.

For example, the amount of the human leukemia inhibitory factor (hLIF) is about 10 ng/ml.

In embodiments, the vessel comprises a dish, a flask, a well, a tube, or a plate. In embodiments, the vessel comprises a solid surface or a porous surface.

In embodiments, the cells are cultured on a surface coated with extracellular matrix or a component of extracellular matrix. For example, the extracellular matrix is Matrigel™ or a Matrigel™-like substance. For example, the surface is not Matrigel™.

Aspects of the invention are also drawn towards an in vitro cell culture. For example, the in vitro cell culture comprises a population of cells derived from a mammalian blastocyst produced by methods as described herein.

In embodiments, the cells of the in vitro cell culture comprise trophoblast stem cells, trophoblast stem cell-like cells, or derivatives.

In embodiments, the cells of the in vitro cell culture comprise undifferentiated cells.

In embodiments, the cells of the in vitro cell culture are capable of self-renewal.

Aspects of the invention are also drawn towards an isolated cell. In embodiments, the isolated cell is derived from a mammalian blastocyst.

In embodiments, the isolated cell expresses at least one marker of pluripotency. For example, the at least one marker comprises GATA3, CDX2, ELF3, TFAP2A, KLF5, KRT8, SFN, DNMT1, DNMT3A, PAG2, PAG11, PAG12, CYP17A1, HSD3B1, HAND1, or any combination thereof. For example, the at least one marker comprises a marker of the Wnt signaling pathway, the LIF signaling pathway, the HIF-1 signaling pathway, the AMPK signaling pathway, or any combination thereof.

In embodiments, the isolated cell comprises a trophoblast stem cell, a trophoblast stem cell-like cell, or a derivative thereof.

In embodiments, the isolated cell is undifferentiated.

In embodiments, the isolated cell is capable of self-renewal.

In embodiments, the isolated cell is capable of differentiation into cells of the trophoblast lineage in vitro and in vivo.

In embodiments, the isolated cell is a bovine cell.

Aspects of the invention are further drawn towards a method of evaluating a candidate compound.

In embodiments, the method comprises contacting an in vitro cell culture or an isolated cell as described herein with an amount of the candidate compound and evaluating a characteristic of the in vitro cell culture or isolated cell. For example, the characteristic is cell growth, cell development, differentiation, apoptosis, trophoblast development, trophoblast activity, or any combination thereof.

Still further, aspects of the invention are drawn towards a cell culture comprising a population of bovine embryonic stem cells in a medium.

In embodiments, the medium comprises one or more factors selected from the group consisting of a human leukemia inhibitory factor (hLIF), an inhibitor of glycogen synthase kinase-3 (GSK-3), an antagonist of muscarinic M2 and histamine H1 receptors, and an inhibitor of matrix metalloproteinase (MMP), or any combination thereof.

For example, the amount of the human leukemia inhibitory factor (hLIF) is about 10 ng/ml.

For example, the amount of the glycogen synthase kinase-3 inhibitor is about 3 μM.

For example, the amount of the muscarinic M2 and histamine H1 receptor antagonist is about 2 μM.

For example, the amount of the matrix metalloproteinase inhibitor is about 2 μM.

In embodiments, the cell culture is in a microwell plate.

In embodiments, the cell culture further comprises a population of trophoblast stem cells. For example, the population of trophoblast stem cells comprises a population of bovine trophoblast stem cells.

Other objects and advantages of this invention will become readily apparent from the ensuing description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 provides a representation showing the derivation and characterization of bovine trophoblast stem cells (bTSC) in vitro. Panel A provides a schematic diagram representing derivation of bovine TSC from blastocyst. Panel B provides bright field images of the outgrowths of blastocysts and typical morphologies of bovine TSC on feeder or feeder-free. D5: outgrowth after 5 days culture; P15: passage 15; P60: passage 60. (Scale bar: 100 μm) Panel C provides a representation showing immunofluorescence staining for GATA3, KRT8, CDX2 and SOX2 in bovine Day 7 IVF blastocysts and bTSC. (Scale bar: 50 μm.) Panel D provides RT-PCR analysis of expression of CDX2, SFN, ELF5, GATA3, ASCL2, GATA2, ETS2 and GAPDH in bovine TSC. BEF: bovine embryonic fibroblast; bESC: bovine embryonic stem cells. Panel E provides a representation showing flow cytometry analysis of GATA3 in bTSC.

FIG. 2 provides a representation showing bovine TSC in vitro developmental potential. Panel A provides a representation of immunofluorescent (IF) images showing binucleation in differentiated-bTSC (P27). (Scale bar: 100 μm) Panel B provides a representation showing IF staining images of differentiated-bTSC (P27) for PTGS2 and PL-1. (Scale bar: 75 μm) Panel C provides a representation showing IFNT activity secreted by bTSC and trophoblast lineages (from Day 2 to Day 6 of bTSC in vitro differentiation), n=6. IFNT: interferon tau. Panel D provides a representation showing expression levels of IFNT in bTSC and during in vitro differentiation. Panel E provides a representation showing expression levels of binuclear trophoblast markers: BEVR-k1 env, ERVE-A and PAG1. Panel F provides a representation showing expression levels of trophoblast markers: PAG2, PAG11 and PAG12 according to RNAseq data. Panel G provides a representation showing gene expression dynamics of functional trophoblast cells markers: CYP11A1, CYP17A1, FURIN, HAND1, PTGS2 and HSD3B1 during differentiation according to RNAseq data. Panel H provides a representation showing top 10 enriched gene ontology (GO) terms in Diff_D4 trophoblast compared with bTSC. Panel I provides a representation showing gene set enrichment analysis (GSEA) of bTSC and Diff_D4 trophoblast cells. Green line shows enrichment profile. Vertical black bars show where genes from a given gene set are located. Data are presented as the mean±SD of three independent experiments. *P<0.05, **P<0.01, ***P<0.001.

FIG. 3 provides a representation showing engraftment of bTSCs into NOD-SCID mice. Panel A provides a representation showing NOD-SCID mice with tumor formed after bTSCs were injected. (Left) Tumors removed from mice after 9 days injection. (Right) Panel B provides a representation showing hematoxylin and eosin (H&E) staining in TS-derived lesion. Asterisk: necrotic area. (Scale bar: 200 μm) (Panel C) Blood-filled lacunae (arrow). (Panel D) Binucleate cells. (Panel E) IF images stained for MMP2, PL-1 and PTGS2. (Scale bar: 75 μm).

FIG. 4 provides a representation showing transcriptomic features of bovine TSC. Panel A provides a representation showing principal-component analysis (PCA) of global gene expression (RNA-seq) of bTSC, trophectoderm of day 7 IVF blastocyst (D7_TE), day 7 IVF blastocyst (BL), trophoblast from day 14 elongated embryos (D14_TE), bESC and bEPSC. Panel B provides a representation showing a PCA plot showing RNA-seq data from mouse ESC/TSC, human ESC/TSC and bovine ESC/TSC. Panel C provides a representation showing expression pattern of trophoblast and pluripotency marker genes in bTSC, D7_TE, BL, D14_TE, bESC and bEPSC. Panel D provides a representation showing a heatmap of highly expressed genes in bTSC. Panel E provides a representation showing KEGG pathway enriched in highly expressed genes in bTSC. Panel F provides a representation showing Top 5 enriched and depleted GO terms in bTSC compared with D7_TE or D14_TE. Panel G provides a representation showing GSEA comparison between bTSC and bESC and bEPSC^{ES}. Genes with Hippo signaling pathway, lysosome and Tight junction were upregulated in bTSC.

FIG. 5 provides a representation showing ATAC-seq-based chromatin accessibility of bTSCs. Panel A provides a representation showing a sample Pearson correlation analysis for bTSC, D7_TE, D14_TE and Diff_D4 trophoblast cells. Panel B provides a representation showing the enrichment of ATAC-seq peaks at transcription start sites (TSS) in bTSC, D7 TE and D14_TE. Panel C provides a representation showing enriched motifs of bTSC. Panel D provides a representation showing genomic views of selected genes according to ATAC-seq data in bTSC, D7_TE and D14_TE. Panel E and Panel F show enriched and depleted KEGG in bTSC compared with D7_TE (Panel E) or D14_TE (Panel F) according to chromatin accessibility.

FIG. 6 provides a representation showing DNA methylome profiling of bTSCs. Panel A provides a representation showing Pearson correlation coefficients for comparing DNA methylation levels between bTSC, D7_TE, D14_TE and bEPSC. Panel B provides a representation showing DNA methylation levels in bTSC, D7_TE, D14_TE and bEPSC. (bTSC, D7_TE and bEPSC: 2 replicates; D14_TE: 3 replicates). Panel C provides a representation showing Methylation levels at genomic features: promoter, exon, intron and intergenic in bTSC, D7_TE, D14_TE and bEPSC. Panel D provides a representation showing expression of DNA methylation genes in bTSC, D7_TE, D14_TE and bEPSC. Panel E provides a representation showing numbers of DMRs and corresponding genes between different groups. Panel F and Panel G provide a representation enriched KEGG of hypermethylated DMRs in D7_TE (Panel F) and D14_TE (Panel G).

FIG. 7 provides a representation showing LCDM supports bESCs. Panel A provides a schematic diagram showing the transition of primed bESC into LCDM-ESC. (Scale bar: 100 μm). Panel B provides a representation showing IF staining images of SOX2, NANOG, CDX2 and GATA3 in LCDM-ESC, primed ESC and bTSC. (Scale bar: 75 μm). Panel C provides a representation showing PCA analysis of bovine inner cell mass (ICM), ESC, EPSCES, EPSCiPS and TSC. Panel D provides a representation showing Left: Venn diagram of upregulated genes in three groups. Right: Top 10 GO terms of upregulated genes in LCDM-ESC. Panel E provides a representation showing Left: Heatmap of specific genes in bESC, bEPSC^{ES}, LCDM-ESC and bTSC respectively. Right: enriched GO terms of LCDM-ESC specific genes.

FIG. 8. Panel A provides a representation showing a screening of basal mediums, growth factors and inhibitors required for bovine TSC in vitro and outgrowth rate for the media. C1: combination 1; DM: Dimethindene maleate; MH: Minocycline hydrochloride. Panel B provides a representation showing the outgrowths of blastocysts after 7 days culture (top row) and Passage 3 (P3) cells (bottom row) in C9, C10 and C11 mediums. (Scale bar: 100 μm.)

FIG. 9 Panel A provides a representation showing IF analysis of GATA3, KRT8, CDX2 and SOX2 in bTSC at Passage 10 (P10) and Passage 55 (P55). (Scale bar: 50 μm.) Panel B provides a representation showing the karyotype for bTSC at passage 15 and 45, respectively. Panel C provides a representation showing bright field image of differentiated-TSC. (Scale bar: 50 μm.) Panel D provides a representation of IF images showing binucleation in differentiated-bTSC (P55). (Scale bar: 25 μm) Panel E and Panel F provide a representation showing IF staining images of differentiated-bTSC (P55) for PL-1 and PTGS2. (Scale bar: 25 μm)

FIG. 10 Panel A provides a representation showing PCA of global gene expression (RNA-seq) of bTSC and differentiated-TSC. Panel B provides a representation showing Pearson correlation analysis for bTSC and differentiated-TSC. Panel C provides a representation showing Pearson correlation analysis for bTSC, Day 7 blastocyst (BL), D7_TE and D14_TE. Panel D provides a representation showing differential expression profile between bTSC and Diff_D4. Panel E provides a representation showing GO analysis for top 50 upregulated genes in Diff_D4.

FIG. 11 Panel A provides a representation showing expression levels of SOX2, OCT4 and NANOG in bESC and LCDM-ESC. Panel B provides a representation showing relative expression levels of tight-junction-related Claudin family genes in bESC and LCDM-ESC. Panel C and Panel D provide a representation showing transcriptome analysis of selected primed (Panel C) and naïve (Panel D) pluripotency markers in bESC and LCDM-ESC (bESC: 2 replicates; LCDM-ESC: 3 replicates). Data are presented as the mean±SD of three independent experiments. *P<0.05, **P<0.01, ***P<0.001.

FIG. 12 provides a representation showing the derivation and characterization of bovine TSCs. Panel A provides an illustration of the derivation of bTSCs from blastocysts. Panel B provides bright-field images of the outgrowths of blastocysts and typical morphologies of bTSCs on feeder or feeder free. D7, outgrowth after 7 days of culture; P15, passage 15; P60, passage 60. Scale bar: 100 μm. Panel C provides immunostaining for epiblast marker SOX2 and trophoblast markers (GATA3, KRT8, and CDX2) in bovine day 7 IVF blastocysts and bTSCs. Scale bar: 50 μm. Panel D provides flow cytometry quantification of GATA3+ cell population in bTSCs. Panel E provides immunostaining for bovine mature trophoblast markers (PL-1 and PTGS2) in differentiated-bTSCs (P55). Arrow: binucleate cells. Scale bar: 25 μm. Panel F provides a graph showing IFNτ activity secreted by bTSCs and trophoblast cells differentiated from bTSCs from day 2 to day 6 (n=6). IFNτ, interferon t. Panel G provides graphs showing expression levels of IFNτ, ERVE-A, BEVR-k1 env, and PAG1 during in vitro differentiation (n=3). Panel H provides a representation showing the expression dynamics of mature trophoblast cell marker genes (PAG1, PAG11, PAG12, CYP17A1, HAND1, PTGS2, CYP11A1, FURIN, and HSD3B1) during bTSC in vitro differentiation (n=2). Panel I provides H&E staining analysis of bovine TSC-derived lesion. Asterisk: necrotic area; arrow: blood-filled lacunae; arrowhead: binucleate cells. Scale bar: 200 μm. Panel J provides immunostaining for mature trophoblast markers (PL-1 and PTGS2) and trophoblast-endometrial regulator (MMP2) in TSC-derived lesion. Scale bar: 75 μm. (Panel F and Panel G) Data are presented as the mean±SD. *p<0.001, **p<0.0001, ***p<0.00001, ****p<0.000001.

FIG. 13 provides a representation showing transcriptomic features of bovine TSCs. Panel A provides principal-component analysis (PCA) of global gene expression (RNA-seq) of bTSCs, trophectoderm of day 7 IVF blastocysts (D7_TE), day 7 IVF blastocysts (BLs), trophoblast from day 14 elongated embryos (D14_TE), bESCs, and bEPSCs. Panel B provides a PCA plot showing RNA-seq data from mouse ESCs/TSCs, human ESCs/TSCs, and bovine ESCs/TSCs. Panel C provides an expression pattern of trophoblast and pluripotency marker genes in bTSCs, D7_TE, BLs, D14_TE, bESCs, and bEPSCs. Panel D provides GSEA of transcriptomes between bTSCs, bESCs, and bEPSCs^{ES}. Genes with Hippo signaling pathway and tight junction were upregulated in bTSCs. Panel E provides a heatmap of highly expressed genes in bTSCs (left). Enriched KEGG of bTSCs' highly expressed genes (right). Panel F and Panel G provide representations showing the top 7 enriched and depleted KEGG in bTSCs compared with bEPSCs^{Xiang} (Panel F) or bEPSCs^{ES} (Panel G).

FIG. 14 provides a representation showing epigenomic features of bovine TSCs. Panel A provides a motif enrichment analysis of ATAC-seq peaks from bTSCs. Panel B provides a representation showing the pathways enriched in genes with more accessible or closed chromatin in bTSC compared with D7_TE. Panel C provides a graph showing the average genome-wide DNA methylation levels of bTSCs, D7_TE, D14_TE, and bEPSCs (bTSCs, D7_TE, and bEPSCs: n=2; D14_TE: n=3). Panel D provides a graph showing the expression levels of DNA methyltransferase (DNMT1, DNMT3A, and DNMT3B) in bTSCs, D7_TE, D14_TE, and bEPSCs. Panel E provides a representation showing the total number of identified differentially methylated regions (DMRs) and their annotated genes between bTSCs and D7_TE or D14_TE. Panel F and Panel G provides representations showing enriched pathways associated with genes annotated from hypomethylated DMRs in bTSCs compared to D7_TE (Panel F) or D14_TE (Panel G).

FIG. 15 provides a representation showing the characterization of bovine TSCs. Panel A provides representative images of the outgrowths of blastocysts after 7 days culture (top row) and cells after 3 passages (P3) (bottom row) in C9, C10 and C11 medium. Scale bar: 100 μm. Panel B provides karyotyping of bTSCs at passage 15 and 45, respectively. Panel C provides RT-PCR analysis of bovine trophoblast marker genes (CDX2, SFN, ELF5, GATA3, ASCL2, GATA2, and ETS2) in bovine TSCs. GAPDH serves as control. BEF: bovine embryonic fibroblast; bESC: bovine embryonic stem cells. Panel D provides immunostaining for epiblast marker SOX2, and trophoblast marker (GATA3, KRT8, CDX2) in bTSCs at passage 10 (P10) and passage 55 (P55) (Scale bar: 50 μm). Panel E provides a bright field image of differentiated-TSCs. Scale bar: 50 μm. Panel F provides representative immunostaining images showing binucleation in differentiated-bTSCs (P27). Scale bar: 100 μm. Panel G provides representative immunostaining of mature trophoblast markers (PTGS2 and PL-1) in differentiated-bTSCs (P27). Scale bar: 75 μm.

FIG. 16 provides a representation showing transcriptomic and epigenetic features of bovine TSCs. Panel A provides PCA analysis of transcriptomes of bTSC, D7_TE, D14_TE and differentiated-TSCs at day 2, 3, 4, 5, and 6. Panel B provides a representation showing the top 10 enriched gene ontology (GO) terms in Diff_D4 trophoblast compared with bTSCs. Panel C provides gene set enrichment analysis (GSEA) of bTSC and Diff_D4 trophoblast cells. Green line shows enrichment profile. Vertical black bars show where genes from a given gene set are located. Panel D provides a representation showing the enriched GO terms of upregulated genes in Diff_D5 or Diff_D6. Panel E provides a representation showing NOD-SCID mice with tumor formed after bTSC were injected (Top row). Tumors removed from mice after 9 days injection (Bottom row). Panel F provides a representation showing the top 5 enriched and depleted GO terms in bTSC compared to D7_TE or D14_TE. Panel G provides a representation showing pathways enriched in genes with more accessible or closed chromatin in bTSC compared to D14_TE. Panel H and Panel I provide representations showing enriched pathways associated with genes annotated from hypermethylated DMRs in bTSCs compared to D7_TE (Panel H) or D14_TE (Panel I).

FIG. 17 provides a schematic showing that bovine trophoblast stem cells (bTSCs) retain developmental potency to differentiate into mature trophoblast cells and exhibit transcriptomic and epigenetic features characteristic of trophectoderm cells from early bovine embryos.

### DETAILED DESCRIPTION OF THE INVENTION

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b, and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Aspects of the invention are drawn to methods of culturing, expanding, or growing a population of cells derived from a mammalian blastocyst. Once fertilized, a zygote travels down the fallopian tube and mitotically divides many times to form a population of cells called a blastocyst. The blastocyst consists of an inner mass that develops into the embryo, while the outer layer develops into tissue that nourishes and protects the embryo. The blastocyst attaches onto the wall of the uterus and receives nourishment through the mother's blood. The major systems structures of the calf develop during the embryonic period in a process called differentiation. During this stage, kidney, brain, spinal cord, nerve, heart, and blood cells start to develop, and the gastrointestinal tract begins to form.

Trophoblasts are cells that form the outer layer of a blastocyst. They provide nutrients to the embryo and develop into a large part of the placenta. They form during the first stage of pregnancy and are the first cells to differentiate from the fertilized egg to become extraembryonic structures and do not directly contribute to the embryo.

Embodiments as described herein comprise culturing cells derived from a mammalian blastocyst, for example, a bovine blastocyst. “Culturing” a cell or a population of cells can refer to propagating or nurturing a cell, collection of cells, tissue, or organ, by incubating for a period of time in an environment and under conditions which support cell viability or propagation. For example, culturing a cell can maintain the cell under conditions in which it can proliferate, differentiate, and avoid senescence. For example, the environment and conditions which support cell viability and/or propagation can include culturing a cell or a population of cells in a culture medium comprising human leukemia inhibitory factor (hLIF), an inhibitor of glycogen synthase kinase-3 (GSK-3), an antagonist of muscarinic M2 and histamine H1 receptors, an inhibitor of matrix metalloproteinase (MMP), or any combination thereof.

Culturing can include one or more of the steps of expanding and proliferating a cell or population of cells, and/or collecting a cell, a population of cells, a tissue, or and organ.

“Expanding” a population of cells can refer to culturing the cells for a period of time and under conditions that not only allow the cells to grow and develop, but also proliferate, so that at the end of the expansion, more cells are obtained than before the expansion. For example, one cell can be expanded by cell division to two cells. In embodiments, expansion of a population of cells can occur spontaneously as certain cells proliferate in a culture. In other embodiments, expansion of a population of cells can require certain growth conditions, including but not limited to a minimum cell density, cell confluence on the culture vessel surface, or the addition of chemical factors such as growth factors, differentiation factors, or signaling factors.

As described herein, embodiments comprise culturing cells derived from a mammalian blastocyst. As used herein, “mammal” or “mammalian” can refer to any mammal, non-limiting examples of which include a human, a primate, mouse, rat, dog, cat, bovine, cow, horse, pig, a fish, or a bird. For example, “bovine” can refer to an animal from the cattle group, non-limiting examples of which include cows, buffalo, and bison.

In embodiments, the method described herein comprises culturing mammalian cells in a culture medium. The terms “medium”, “cell culture medium”, “culture medium” can refer to a solution containing nutrients that nourish growing cells. In certain embodiments, the culture medium is useful for growing mammalian cells. A culture medium can provide essential and non-essential amino acids, vitamins, energy sources, lipids, and trace elements required by the cell for minimal growth and/or survival. A culture medium can also contain supplementary components (see discussion of “Supplementary components” below) that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds present at very low final concentrations), amino acids, lipids, and/or glucose or other energy source. In certain embodiments, a medium is advantageously formulated to a pH and salt concentration optimal for cell survival and proliferation.

In embodiments, the culture medium can comprise “supplementary components”, which can refer to components that enhance growth and/or survival above the minimal rate. Non-limiting examples of supplementary components include hormones and/or other growth factors, ions (such as sodium, chloride, calcium, magnesium, and phosphate), buffers, vitamins, nucleosides or nucleotides, trace elements (inorganic compounds present at very low final concentrations), amino acids, lipids, and/or glucose or other energy source. In certain embodiments, supplementary components are added to the initial cell culture. In certain embodiments, supplementary components are added after the beginning of the cell culture.

The term “defined medium” can refer to a medium in which the composition of the medium is both known and controlled. See, for example, the medium of Example 4.

In embodiments, the cell culture can comprise a “nutrient source”, which can refer to a composition, including the source itself, that nourishes growing mammalian cells. Non-limiting examples of nutrient sources comprise DMEM, IDMEM, MEM, M199, RPMI 1640, Ham's F12, DMEM/F12, Ham's F10, McCoy's 5A, NCTC 109, and NCTC 135.

A “culture medium” can refer to a solution for growing, storing, handling and maintaining a cell, a population of cells, and/or cell lines. In embodiments, solutions can include factors required for or assist with cell attachment, cell growth, cell proliferation, maintenance of a cell in an undifferentiated state, and/or maintenance of the cellular environment. Non-limiting examples of such factors include salts, nutrients, minerals, vitamins, amino acids, nucleic acids, proteins such as cytokines, growth factors and hormones.

In embodiments, the culture medium comprises a human leukemia inhibitory factor, an inhibitor of glycogen synthase kinase-3, an antagonist of muscarinic M2 and histamine H1 receptors, an inhibitor of matrix metalloproteinase, or any combination thereof.

In embodiments, the culture medium can be a liquid solution that supports the growth of stem cells, such as trophoblast stem cells, and/or maintains them in an undifferentiated state. For example, a “culture of mammalian cells” can refer to a liquid culture medium containing a plurality of mammalian cells that is maintained or proliferated under a controlled set of physical conditions. In embodiments, the culture medium can be a water-based medium.

A “cell culture” can refer to cells growing in suspension or adhered to a variety of surfaces or substrates in a vessel, such as a roller bottle, tissue culture flask, dish, multi-well plate, and the like. For example, the cell culture can refer to a population of cells, such as trophoblast stem cells, derived from a mammalian blastocyst and a culture medium comprising human leukemia inhibitory factor (hLIF), an inhibitor of glycogen synthase kinase-3 (GSK-3), an antagonist of muscarinic M2 and histamine H1 receptors, an inhibitor of matrix metalloproteinase (MMP), or any combination thereof.

In embodiments, the cell culture can comprise a plurality of cells in an undifferentiated state. For example, a cell in an “undifferentiated state” can refer to a cell that does not have specialized structures or functions. Thus, an undifferentiated cell can refer to a cell having differentiation ability that has not entered the process of differentiating into a cell having a specific function as a tissue or organ. In embodiments, the undifferentiated cells are capable of self-renewal.

In embodiments, the cell culture can comprise a plurality of cells capable of differentiation. “Differentiation” or “to differentiate” can refer to the process by which a less specialized cell (e.g., stem cells, embryonic cells) undergoes maturation to become more distinct in form and function, such as to acquire specialized structural and/or functional features characteristic of mature cells. For example, the less specialized cell (e.g., a stem cell or cell maintaining stemness) can progress from the stage of having the potential to differentiate into a cell of different cellular lineages to the stage of becoming a specialized and terminally differentiated cell. During differentiation, cellular structure alters and tissue-specific proteins appear.

Non-limiting examples of undifferentiated cells include pluripotent stem cells, embryonic stem cells, progenitor cells, induced pluripotent stem cells, germ stem cells, and the like.

Embodiments as described herein provide methods of culturing a population of cells derived from a mammalian blastocyst, wherein the population of cells maintain cell viability. “Cell viability” can refer to the ability of cells in culture to survive under a given set of culture conditions or experimental variations. The term can also refer to that portion of cells which are alive at a particular time in relation to the total number of cells, living and dead, in the culture at that time.

In embodiments, the method as described herein can comprise placing a mammalian blastocyst in a vessel, thereby providing a vessel comprising a population of cells derived from a mammalian blastocyst. Any suitable vessel can be used in the embodiments described herein. For example, a “vessel” can refer to a type of culture ware which provides a contamination barrier to protect the cultures from the external environment while maintaining the proper internal environment. Non-limiting examples of a vessel comprise a flask, a tube, a Petri dish, a roller bottle, and/or a multi-well plate.

For example, the population of cells derived from a mammalian blastocyst, such as a population of trophoblast stem cells, can be cultured in a microwell plate. In some cases, the microwell plate is a v-bottomed microwell plate. In some cases, the v-bottomed microwell plate is an AggreWell plate.

In embodiments, culturing a population of cells as described herein can comprise centrifuging a culture vessel comprising the population of cells and the culture media. For example, a culture vessel can be centrifuged at about 50×g after the cell and medium is added to the plate. For example, a culture vessel can be centrifuged at about 100×g after the cell and medium is added to the plate. For example, a culture vessel can be centrifuged at about 150×g after the cell and medium is added to the plate. For example, a culture vessel can be centrifuged at about 200×g after the cell and medium is added to the plate. For example, a culture vessel can be centrifuged at about 250×g after the cell and medium is added to the plate. For example, a culture vessel can be centrifuged at about 300×g after the cell and medium is added to the plate. For example, a culture vessel can be centrifuged at about 350×g after the cell and medium is added to the plate. For example, a culture vessel can be centrifuged at about 400×g after the cell and medium is added to the plate. For example, a culture vessel can be at about 450×g after the cell and medium is added to the plate. In some cases, the v-bottomed plate is centrifuged at about 500×g after the cell and medium is added to the plate.

In embodiments, the vessel can be seeded with fibroblast cells, such as mouse embryonic fibroblast cells. “Cell seeding” can refer to spreading cells into or onto a surface of a vessel. “Fibroblast cells” can refer to a cell that contributes to the formation of connective tissue and are not terminally differentiated. Fibroblasts are heterogeneous mesenchymal cells that play important roles in the production and maintenance of extracellular matrix.

In embodiments, the trophoblast stem cells can be cultured with or without fibroblast feeder cells. A “fibroblast feeder cell” can refer to a cell of one type that can be cultured with a cell of another type to provide an environment in which the cell of the second type can grow. For example, a trophoblast stem cell can be cultured with a MEF such that the trophoblast stem cell grows. The feeder cell can be a human feeder cell or can be a nonhuman feeder cell. In one embodiment, the feeder cell can be a mouse embryonic fibroblast.

The phrase “feeder cell support” as used herein refers to the ability of a feeder cell (e.g., fibroblasts) to maintain pluripotent stem cells in a proliferative and undifferentiated state when the pluripotent stem cells are co-cultured on the feeder cells or when the pluripotent stem cells are cultured on a matrix (e.g., an extracellular matrix, a synthetic matrix) in the presence of a conditioned medium generated by the feeder cells. The support of the feeder cells depends on the structure of the feeder cells while in culture (e.g., the three dimensional matrix formed by culturing the feeder cells in a tissue culture plate), function of the feeder cells (e.g., the secretion of growth factors, nutrients and hormones by the feeder cells, the growth rate of the feeder cells, the expansion ability of the feeder cells before senescence) and/or the attachment of the pluripotent stem cells to the feeder cell layer(s).

The phrase “absence of feeder cell support” as used herein refers to a culture medium and/or a cell culture being devoid of feeder cells and/or a conditioned medium generated thereby.

In embodiments, the vessel can comprise a “surface” to which the cell or population of cells can attach. For example, the surface can be a solid substrate, a porous substrate, or another non-solid substrate.

For example, the solid surface can be coated with an insoluble substrate that, optionally, can in turn be coated with one or more additional surface coats of a substrate, or any other chemical or biological material that allows the cells to proliferate or be stabilized in culture. Non-limiting examples of a substrate comprise any one or combination of polyornithine, laminin, poly-lysine, purified collagen, gelatin, fibronectin, tenascin, vitronectin, entactin, heparin sulfate proteoglycans, polyglycolytic acid (PGA), polylactic acid (PLA), and polylactic-glycolic acid (PLGA).

A “porous surface”, for example, can refer to a surface which allows for the partitioning of cellular microenvironments in vitro, while still allowing physical and biochemical crosstalk between cells.

In embodiments, the cells can be cultured on a surface coated with extracellular matrix or a component of extracellular matrix as described herein.

As described herein, aspects of the invention are drawn towards culturing a population of cells derived from a mammalian blastocyst. The terms “cell” and “population of cells” can refer to a plurality of cells (i.e., more than one cell). In embodiments, the population can be a pure population comprising one cell type. In other embodiments, the population can include multiple cell types. Accordingly, there is no limitation on the types of cells that the population of cells can contain. In embodiments, the population of cells can comprise trophoblast stem cells or trophoblast stem-like cells.

Any suitable population of cells can be used in methods for culturing, expanding, or growing a population of cells derived from a mammalian blastocyst, such as trophoblast stem cells as described herein. In embodiments, the population of cells can comprise reproductive cells, e.g., female germline stem cells and progeny thereof. Examples of reproductive cells include, but are not limited to, embryos, oocytes, zygotes, blastomeres, morulae, and blastocysts.

In embodiments, the population of cells can comprise somatic cells such as fibroblasts (e.g., embryonic fibroblasts or skin fibroblasts). Somatic cells can be obtained by well-known methods from different organs, e.g., skin, lung, pancreas, liver, stomach, intestine, heart, reproductive organs, bladder, kidney, urethra, and other urinary organs. Examples of somatic cells include, but are not limited to, adult stem cells, Sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B lymphocytes and T lymphocytes), erythrocytes, macrophages, monocytes, mononuclear cells, fibroblasts, cardiac muscle cells, and other muscle cells.

The suitable population of cells can be obtained from any suitable source. In embodiments, the population of cells can be obtained from a subject, for example, from tissue (e.g., embryotic tissue), bone (e.g., bone marrow), blood (e.g., peripheral blood or umbilical cord blood), bodily fluid (e.g., tear, urine, or saliva), serum, plasma, or protein, from a subject via any means known in the art. A subject includes, but is not limited to, a human or a nonhuman mammal such as a rodent (e.g., a mouse or a rat), an ungulate (e.g., a horse or a pig), or bovine (e.g., cow).

A “stem cell” can refer to an undifferentiated cell which is capable of essentially unlimited propagation in vivo or ex vivo and capable of differentiation to other cell types. This can be to certain differentiated, committed, immature, progenitor, or mature cell types present in the tissue from which it was isolated, or dramatically differentiated cell types that derive from a common precursor cell, or even to cell types at any stage in a tissue completely different from the tissue from which the stem cell is obtained. A stem cell can retain a constant potential for differentiation even after undergoing cell division. Examples of the stem cells include embryonic stem cells (ES cells) with pluripotency derived from a fertilized egg or a clone embryo, somatic stem cells and pluripotent stem cells that are present in tissues in a live body, hepatic stem cells, dermal stem cells, and germ stem cells that serve as the bases for respective tissues, pluripotent stem cells derived from a germ stem cell, pluripotent stem cells derived from a somatic cell that are obtained by nuclear reprogramming, and the like.

A “stem-like cell” can refer to cells that have some of the characteristics of stem cells. For example, they have some ability to self-renew. Examples of stem-like cells include, but are not limited to, progenitor cells, multipotent stem cells, cells undergoing process to induce pluripotency, cancer cells, cancer stem cells, hematopoietic stem cells, iPS, and some antibody producing hybridoma cells.

A “trophoblast stem cell” can refer to the precursor of the differentiated cells of the placenta, which mediate the interactions between the fetus and the mother.

A “pluripotent stem cell” can refer to a stem cell that permits cultivation in vitro. A pluripotent stem cell can differentiate into cells constituting the body. A “pluripotent stem cell” can be obtained from a fertilized egg, a clone embryo, a germ stem cell, or a stem cell in a tissue. Also included are cells having differentiation pluripotency similar to that of embryonic stem cells, conferred artificially by transferring several different genes to a somatic cell.

An “embryonic stem cell” can refer to a cell which is obtained from the embryonic tissue formed after gestation (e.g., blastocyst) before implantation (i.e., a pre-implantation blastocyst), extended blastocyst cells (EBCs) which are obtained from a post-implantation/pre-gastrulation stage blastocyst, and embryonic germ (EG) cells which are obtained from the genital tissue of a fetus any time during gestation, preferably before 10 weeks of gestation. A “fetus” can refer to a mammal in the developmental stage after the embryonic stage and before birth, with fully differentiated but not yet fully grown organs.

Examples of stem cells that can be used in embodiments described herein include mammalian embryonic stem cells or the like established by culturing a pre-implantation early embryo, embryonic stem cells established by culturing an early embryo prepared by nuclear-transplanting the nucleus of a somatic cell, trophoblast stem cells established from various species, including bovine, mouse, human, and nonhuman primates, and induced pluripotent stem cells (iPS cells) established by transferring several different genes to a somatic cell.

In embodiments, the cells are cultured on a surface coated with extracellular matrix or a component of extracellular matrix (ECM). For example, the ECM can be composed of a variety of polysaccharides, water, elastin, and glycoproteins. Non-limiting examples of glycoproteins can comprise collagen, entactin (nidogen), fibronectin, and laminin. ECM can be secreted by connective tissue cells. Different types of ECM are known, each of which comprise different compositions including different types of glycoproteins and/or different combination of glycoproteins. ECM can be provided by culturing ECM-producing cells, for example fibroblast cells, in a vessel prior to the removal of these cells and the addition of isolated tissue fragments or isolated epithelial stem cells, such as a mammalian blastocyst.

Non-limiting examples of extracellular matrix-producing cells comprise chondrocytes, producing mainly collagen and proteoglycans; fibroblast cells, producing mainly type IV collagen; laminin; interstitial procollagens; fibronectin; colonic myofibroblasts producing mainly collagens (type I, III, and V); chondroitin sulfate proteoglycan; hyaluronic acid; fibronectin; and tenascin-C.

In embodiments, ECM can be commercially provided. Non-limiting examples of commercially available extracellular matrices comprise extracellular matrix proteins (Invitrogen) and basement membrane preparations from Engelbreth-Holm-Swarm (EHS) mouse sarcoma cells (e.g., MATRIGEL™ (BD Biosciences)). A synthetic extracellular matrix material, such as ProNectin (Sigma Z378666) can be used. Mixtures of extracellular matrix materials can be used.

In embodiments, the use of an ECM for culturing stem cells can enhance the long-term survival of the stem cells and the continued presence of undifferentiated stem cells.

Aspects of the invention can be further drawn to an in vitro cell culture comprising a population of cells derived from a mammalian blastocyst and a medium as described herein. In embodiments, the in vitro population of cells are capable of self-renewal. “Self-renewal” can refer to the process by which stem cells perpetuate themselves, such as to replenish mature cells to maintain tissue homeostasis throughout the lifespan of an organism. Self-renewal is division with maintenance of the undifferentiated state. This can require cell cycle control and/or maintenance of multipotency or pluripotency, depending on the stem cell.

Aspects of the invention are further drawn to an isolated cell derived from a mammalian blastocyst. An “isolated cell” can refer to a cell that has been removed from its natural environment, for example, as a part of an organ, tissue, or organism. Markers (i.e., biomarkers) can be used to identify and isolate different cell types. For example, embodiments as described herein can comprise an isolated cell expressing at least one marker of pluripotency. “Pluripotency” can refer to the ability of a cell to develop into the body or lineages of the body (i.e., embryo body). For example, a pluripotent cell can develop into the three primary germ cell layers of the early embryo, and therefore into cells of the adult body. Pluripotent stem cells can undergo self-renewal and give rise to cells of the tissues of the body. Non-limiting examples of a marker of pluripotency comprise CDX2, GATA3, ELF3, TFAP2A, KLF5, KRT8, SFN, DNMT1, DNMT3A, PAG2, PAG11, PAG12, CYP17A1, HSD3B1, HAND1, or any combination thereof. Other exemplary markers the isolated cell can express include a marker of a signaling pathway. Non-limiting examples of a signaling pathway comprise Wnt signaling pathway, the LIF signaling pathway, the HIF-1 signaling pathway, the AKT signaling pathway, the AMPK signaling pathway, or any combination thereof.

In other embodiments described herein, the population of cells can comprise at least one marker of a trophoblast stem cell. Referring to Example 1, for example, the resulting cell lines have TSC characteristics, including but not limited to trophoblast marker gene expression, self-renewal, long-term stable morphology, karyotype, and transcriptomic and epigenomic features). Further, the population of cells can contribute to functional uninucleate and binuclear trophoblasts in vitro and in vivo.

Aspects of the invention are also drawn to methods of evaluating a candidate compound. A “candidate compound” can refer to a compound or agent that is to be tested for an activity of interest.

In embodiments, the method comprises contacting the cell culture or the isolated cell as described herein with an amount of the candidate compound, and evaluating a characteristic of the cell culture or isolated cell. Non-limiting examples of characteristics that can be evaluated comprise cell growth, cell development, differentiation, apoptosis, trophoblast development, trophoblast activity, or any combination thereof.

Aspects of the invention further provide a population of cells that can be used for assembly of artificial blastocysts for various Assisted Reproductive Technology (ART) applications. “Assisted reproductive technology” can refer to technology that assists in achieving pregnancy, including, but not limited to, in vitro fertilization (IVF), embryo transfer (e.g., transfer of embryos at any stage, including blastocysts), gamete intrafallopian transfer (GIFT), tubal embryo transfer (TET), intracytoplasmic sperm injection (ICSI) and intrauterine insemination (IUI).

In embodiments, the trophoblast stem cells can be used for the assembly of an artificial blastoid. A “blastoid” can refer to stem cell-based blastocyst-like structures which resemble blastocysts in terms of morphology, size, cell number, and lineage composition and allocation. A “blastocyst” can refer to a thin-walled hollow structure in early embryonic development that contains a cluster of cells called the inner cell mass from which the embryo arises.

As described herein, the culture medium can comprise a human leukemia inhibitory factor, an inhibitor of glycogen synthase kinase-3, an antagonist of muscarinic M2 and histamine H1 receptors, an inhibitor of matrix metalloproteinase, or any combination thereof. Non-limiting examples of the GSK-3 inhibitor include CHIR99021, CHIR98014, CHIR98023, SB-216763 and SB-415286. Non-limiting examples of a muscarinic M2 and histamine H1 receptors antagonist include dimethindene maleate (DiM). Non-limiting examples of an matrix metalloproteinase (MMP) inhibitor includes minocycline hydrochloride (MiH).

In embodiments, the medium comprises two or more factors selected from the group consisting of a human leukemia inhibitory factor (hLIF), an inhibitor of glycogen synthase kinase-3 (GSK-3), an antagonist of muscarinic M2 and histamine H1 receptors, and an inhibitor of matrix metalloproteinase (MMP).

In embodiments, the medium comprises three or more factors selected from the group consisting of a human leukemia inhibitory factor (hLIF), an inhibitor of glycogen synthase kinase-3 (GSK-3), an antagonist of muscarinic M2 and histamine H1 receptors, and an inhibitor of matrix metalloproteinase (MMP).

In embodiments, the medium comprises a human leukemia inhibitory factor (hLIF), an inhibitor of glycogen synthase kinase-3 (GSK-3), an antagonist of muscarinic M2 and histamine H1 receptors, and an inhibitor of matrix metalloproteinase (MMP). Referring to Example 1, for example, a modified chemical cocktail comprising bLCDM: hLIF, CHIR99021, Dimethindene maleate (DiM), Minocycline hydrochloride (MiH)) allows for the derivation and long-term culture of trophoblast stem cells (TSCs) from a large animal, bovine. In certain embodiments, long-term culture means that cells and/or aggregates of cells can be kept in viable state for durations longer than conventional methods of trophoblast stem cell culture, for example, for over 1 week to 6 weeks or longer.

The methods as described herein comprise culturing a population of cells derived from a mammalian blastocyst for a period of time in a culture medium comprising at least one human leukemia inhibitory factor, at least one inhibitor of glycogen synthase kinase-3 (GSK-3), at least one antagonist of muscarinic M2 and histamine H1 receptors, and at least one inhibitor of matrix metalloproteinase (MMP). For example, the population of cells can be cultured for a period of time sufficient for the assembly of artificial blastocysts. For example, the period of time can be at least 18 hours, at least about 24 hour, at least 36 hours, at least 48 hours, at least 60 hours, at least 72 hours, at least 84 hours, at least 96 hours, at least about 5 days, at least about 6 days, at least about 7 days, at least about 8 days, or longer. In further aspects of methods of culturing trophoblast stem cells, the population of cells is cultured for an appropriate period of time sufficient to form the blastoid. In some cases, the culturing is conducted for about 1 day, about 2 days, about 3 days, about 4 days, about 5 days, about 6 days, about 7 days, about 8 days, about 9 days, about 10 days, or more as needed. In some cases, the culturing is conducted for about 1-5 days, about 2-6 days, about 3-7 days, about 4-8 days, about 5-9 days, or about 6-10 days.

In embodiments, the human leukemia inhibitory factor, glycogen synthase kinase-3 (GSK-3) inhibitor, muscarinic M2 and histamine H1 receptor antagonist, and/or matrix metalloproteinase (MMP) inhibitor can be provided in the culture medium in an effective amount. An “effective amount,” “effective dose,” or an “amount effective to,” as used herein, can refer to an amount of an agent that is effective in providing at least one characteristic of trophoblast stem cells (e.g., cell growth, cell development, differentiation, apoptosis, trophoblast development, trophoblast activity, or any combination thereof). Such characteristics can be monitored by conventional methods or can be monitored according to methods described herein. An effective amount can vary depending on, for example, the human leukemia inhibitory factor, the inhibitor of glycogen synthase kinase-3 (GSK-3), the antagonist of muscarinic M2 and histamine H1 receptors, and the inhibitor of matrix metalloproteinase (MMP) used.

For example, the effective amount of the human leukemia inhibitory factor, the inhibitor of glycogen synthase kinase-3 (GSK-3), the antagonist of muscarinic M2 and histamine H1 receptors, and the inhibitor of matrix metalloproteinase (MMP) used as described herein can result in an increase in the proportion of cells in the formative stage of pluripotency by at least 10% or more, including, e.g., at least about 20%, at least about 30%, at least about 40%, at least about 50%, at least about 60%, at least about 70%, at least about 80%, at least about 90% or more, as compared to the proportion of cells in the formative stage of pluripotency when the population of cells is cultured without the human leukemia inhibitory factor, the inhibitor of glycogen synthase kinase-3 (GSK-3), the antagonist of muscarinic M2 and histamine H1 receptors, and the inhibitor of matrix metalloproteinase (MMP).

In embodiments, an effective amount of a human leukemia inhibitory factor can be between about 0.1 and about 10,000 ng/ml. For example, the effective amount of the human leukemia inhibitory factor can be between about 1 and about 10,000 ng/ml, between about 10 and about 10,000 ng/ml, between about 100 and about 10,000 ng/ml, between about 1,000 and about 10,000 ng/ml, between about 5,000 and about 10,000 ng/ml, between about 0.1 and about 5,000 ng/ml, between about 1 and about 5,000 ng/ml, between about 10 and about 5,000 ng/ml, between about 1,000 and about 5,000 ng/ml, or between about 2,500 and about 5,000 ng/ml. In some embodiments, the effective amount of the human leukemia inhibitory factor for the methods described herein can be between 0.1 and 100 μM. In some embodiments, the effective amount of the human leukemia inhibitory factor for the methods described herein can be between 0.1 and 90 μM, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between 0.1 and 10 μM, between 0.1 and 1 μM, and between 0.1 and 0.5 μM. In some embodiments, the effective amount of the human leukemia inhibitory factor for the methods described herein can be between 0.5 and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 μM, and between 90 and 100 μM.

An effective amount of an inhibitor of glycogen synthase kinase-3 (GSK-3) for the methods described herein can be between 0.1 and 10,000 ng/ml. In some embodiments, the effective amount of the inhibitor of glycogen synthase kinase-3 (GSK-3) for the methods described herein can be between 1 and 10,000 ng/ml, between 10 and 10,000 ng/ml, between 100 and 10,000 ng/ml, between 1,000 and 10,000 ng/ml, between 5,000 and 10,000 ng/ml, between 0.1 and 5,000 ng/ml, between 1 and 5,000 ng/ml, between 10 and 5,000 ng/ml, between 1,000 and 5,000 ng/ml, or between 2,500 and 5,000 ng/ml. In some embodiments, the effective amount of the inhibitor of glycogen synthase kinase-3 (GSK-3) for the methods described herein can be between 0.1 and 100 μM. In some embodiments, the effective amount of the inhibitor of glycogen synthase kinase-3 (GSK-3) for the methods described herein can be between 0.1 and 90 μM, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between 0.1 and 10 μM, between 0.1 and 1 μM, and between 0.1 and 0.5 μM. In some embodiments, the effective amount of the inhibitor of glycogen synthase kinase-3 (GSK-3) for the methods described herein can be between 0.5 and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 μM, and between 90 and 100 μM.

An effective amount of an antagonist of muscarinic M2 and histamine H1 receptors for the methods described herein can be between 0.1 and 10,000 ng/ml. In some embodiments, the effective amount of the antagonist of muscarinic M2 and histamine H1 receptors for the methods described herein can be between 1 and 10,000 ng/ml, between 10 and 10,000 ng/ml, between 100 and 10,000 ng/ml, between 1,000 and 10,000 ng/ml, between 5,000 and 10,000 ng/ml, between 0.1 and 5,000 ng/ml, between 1 and 5,000 ng/ml, between 10 and 5,000 ng/ml, between 1,000 and 5,000 ng/ml, or between 2,500 and 5,000 ng/ml. In some embodiments, the effective amount of the antagonist of muscarinic M2 and histamine H1 receptors for the methods described herein can be between 0.1 and 100 μM. In some embodiments, the effective amount of the antagonist of muscarinic M2 and histamine H1 receptors for the methods described herein can be between 0.1 and 90 μM, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between 0.1 and 10 μM, between 0.1 and 1 μM, and between 0.1 and 0.5 μM. In some embodiments, the effective amount of the antagonist of muscarinic M2 and histamine H1 receptors for the methods described herein can be between 0.5 and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 μM, and between 90 and 100 μM.

An effective amount of an inhibitor of matrix metalloproteinase (MMP) for the methods described herein can be between 0.1 and 10,000 ng/ml. In some embodiments, the effective amount of the inhibitor of matrix metalloproteinase (MMP) for the methods described herein can be between 1 and 10,000 ng/ml, between 10 and 10,000 ng/ml, between 100 and 10,000 ng/ml, between 1,000 and 10,000 ng/ml, between 5,000 and 10,000 ng/ml, between 0.1 and 5,000 ng/ml, between 1 and 5,000 ng/ml, between 10 and 5,000 ng/ml, between 1,000 and 5,000 ng/ml, or between 2,500 and 5,000 ng/ml. In some embodiments, the effective amount of the inhibitor of matrix metalloproteinase (MMP) for the methods described herein can be between 0.1 and 100 μM. In some embodiments, the effective amount of the inhibitor of matrix metalloproteinase (MMP) for the methods described herein can be between 0.1 and 90 M, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between 0.1 and 10 μM, between 0.1 and 1 μM, and between 0.1 and 0.5 μM. In some embodiments, the effective amount of the inhibitor of matrix metalloproteinase (MMP) for the methods described herein can be between 0.5 and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 μM, and between 90 and 100 μM.

In embodiments, the culture medium can be changed after culturing the population of cells for a period of time. For example, the contents of the culture medium can be changed after about 8 hours, about 12 hours, about 16 hours, about 24 hours, about 36 hours, or about 48 hours after culturing the population of cells. In embodiments, the medium is replaced with a medium without the human leukemia inhibitory factor. In embodiments, the medium is replaced with a medium without the GSK-3 inhibitor. In embodiments, the medium is replaced with a medium without the antagonist of muscarinic M2 and histamine H1 receptors. In embodiments, the medium is replaced with a medium without the inhibitor of matrix metalloproteinase.

In embodiments, trophoblast cells as described herein can be used in methods for determining drug toxicity. For example, the method can comprise (a) obtaining or providing a trophoblast cell produced by a method according to any herein described method (b) contacting the trophoblast cell as described herein with the drug; and (c) detecting signs of toxicity.

Methods described herein encompass genetic manipulation of any of the populations of cells described herein. A genetic manipulation includes modifying, inserting, or deleting at least one of the genes in the cells.

Genetic manipulation can include transduction with a vector such as a nonintegrating vector (e.g., an episomal vector) or an integrating vector (e.g., lentiviral vector). In some embodiments, methods described herein involve genetically manipulating a population of cells using an episomal vector. Accordingly, in some embodiments, the population of cells involved in the methods described herein are gene-modified cells.

A “vector,” as used herein is any nucleic acid vehicle (DNA or RNA) capable of facilitating the transfer of a nucleic acid molecule into cells. In general, vectors include, but are not limited to, episomal vectors, plasmids, phagemids, viral vectors, and other vehicles derived from viral or bacterial sources that have been manipulated by the insertion or incorporation of a target nucleotide sequence. Viral vectors include, but are not limited to, vectors comprising nucleotide sequences derived from the genome of the following viruses: retrovirus; lentivirus; adenovirus; adeno-associated virus; SV 40-type viruses; polyomaviruses; Epstein-Barr viruses; papilloma viruses; herpes virus; vaccinia virus; polio virus. One can readily employ other vectors not named but known to the art.

Methods described herein encompass reprogramming the population of cells (e.g., the population of somatic cells) to a less differentiated state. Reprogramming, as used herein, refers to a process that alters or reverses the differentiation status of a cell (e.g., a somatic cell), which can be partially or terminally differentiated. Reprogramming includes complete reversion, as well as partial reversion, of the differentiation status of a cell.

Aspects of the invention also provides for a kit for culturing, expanding, or growing a population of cells derived from a mammalian blastocyst. Non-limiting examples of components of the kit comprise cells, culture media, a vessel, and components as described herein, and instructions for use. The kit can be used to carry out the methods as described herein.

The cells can be packaged in the kit by any suitable means for transporting and storing cells. For example, the cells can be provided in frozen form, such as cryopreserved; dried form, such as lyophilized; or in liquid form, such as in a buffer. Cryopreserved cells, for example, can be viable after thawing.

A culture medium can be included in the kit. For example, the culture medium can comprise essential and non-essential amino acids, vitamins, energy sources, lipids, and trace elements required by the cell for minimal growth and/or survival. The culture medium can comprise a human leukemia inhibitory factor, an inhibitor of glycogen synthase kinase-3, an antagonist of muscarinic M2 and histamine H1 receptors, an inhibitor of matrix metalloproteinase, or any combination thereof. The culture medium can be packaged by any suitable means for transporting and storing media.

The vessel can be a type of culture ware which provides a contamination barrier to protect the cultures from the external environment while maintaining the proper internal environment. For example, a vessel can be a flask, a tube, a Petri dish, a roller bottle, and/or a multi-well plate.

The instructions can include one or more of: a description of the cells of the kit; methods for thawing or preparing cells; culturing schedule; precautions; warnings; and/or references. The instructions can be printed directly on the container (when present), or as a label applied to the container, or as a separate sheet, pamphlet, card, or folder supplied in or with the container. A kit as described herein also includes packaging. In some embodiments, the kit includes a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding cells or medicaments.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Trophoblast stem cells (TSCs) are the precursors of the differentiated cells of the placenta, which mediate the interactions between the fetus and the mother. Here, we show that a modified chemical cocktail (bLCDM: hLIF, CHIR99021, Dimethindene maleate (DiM), Minocycline hydrochloride (MiH)) allows for the derivation and long-term culture of trophoblast stem cells (TSCs) from a large animal, bovine. The resulting cell lines have TSC characteristics (trophoblast marker gene expression, self-renewal, long-term stable morphology, karyotype, and transcriptomic and epigenomic features), and can contribute to functional uninucleate and binuclear trophoblasts in vitro and in vivo. The bovine TSC established here will provide a powerful tool to study placental trophoblast differentiation and function, and early pregnancy failure. Together with embryonic stem cells, the established TSCs and the bLCDM condition can be used for assembly of artificial blastocysts for varies Assisted Reproductive Technology (ART) applications.

Uses of the bovine TSC cell lines include, for example, 1) pregnancy drug screening and testing, 2) used as an in vitro model for basic and translational research on placental development (deposited into ATCC for sale), 3) assembly of artificial bovine blastocysts used for assisted reproductive technologies (in vitro breeding).

Bovine TSCs and conditions to support bovine TSCs are not available to date.

The currently available bovine trophoblast cells including trophoblast cell line (CT-1 and CT-5, BT-1), and more recently an undifferentiated trophectoderm cell with the support of irradiated mouse embryonic fibroblast feeders (MEFs) have been derived from bovine blastocysts. However, none of these cells meet the TSC criteria, i.e., i) are able to maintain long-term self-renewal, and 2) have in vitro and in vivo developmental capacity to the functional trophoblasts. Thus, the generation of self-renewal and stable bovine TSC lines remains unexplored.

### Example 2

Placental trophoblasts play an essential role in communication between the fetus and mother. In the bovine, inadequate placental trophoblast development and subsequent dysfunction results in a range of adverse outcomes in conceptus/offspring; for example, the abnormalities seen in IVF or somatic cell nuclear transfer embryos. The most significant barrier to progress in this field in the bovine is the lesser feasibility of an in vivo experimental system or the lack of manipulatable in vitro cell culture models that recapitulate placental cell differentiation. To date, trophoblast stem cells (TSC) have been established from various species, including mouse, human, and nonhuman primates, but the generation of self-renewal and stable bovine TSC lines remains unexplored. Building upon signaling required for mouse and human TSC pluripotency, we screened 11 culture conditions and found that a culture condition containing a chemical cocktail of human leukemia inhibitory factor (hLIF), CHIR99021 (an inhibitor of glycogen synthase kinase-3 (GSK-3)), DiM (antagonist of muscarinic M2 and histamine H1 receptors), and MiH (inhibitor of matrix metalloproteinase (MMP)) allows for the long-term culture (over 55 passages) of bovine TSC without altered morphology and differentiation from bovine IVF embryos. Three stable cell lines were maintained and used for downstream characterizations (n=3). Real-time quantitative (qRT)-PCR and immunostaining assays showed that resulting cells highly express bovine trophectoderm markers including CDX2, GATA3, and KRT8. These cells had the capacity to differentiate into multinuclear trophoblast cells in vitro that secreted IFN-t and expressed high levels of binuclear trophoblast cell marker genes: PAG2, PAG11, PAG12, and uninucleate trophoblast cell marker genes: CYP17A1, HSD3B1, and HAND1. To validate the pluripotency state of bovine TSCs and to map bovine trophoblast differentiation, we generated transcriptomes and accessible chromatin of in vitro trophoblast cell cultures including TSCs, multinuclear cells from in vitro differentiation at Day 2 and Day 6, and trophoblast lineages including trophectoderm (TE) of Day 7 IVF blastocysts, and trophoblast cells of Day 14 elongating embryos by RNA-seq and assay for transposase-accessible chromatin (ATAC)-seq, respectively. Sequencing analysis was performed with at least two replicates per development stage (n ≥2). Transcriptomes of bovine pluripotent stem cell models that represent epiblasts, prime bovine embryonic stem cells (ESC) and expanded potential stem cells (EPSC) were also compared to identify the bovine trophoblast stem niche. Our analysis revealed important transcriptional factors (e.g., GATA3, ELF3, TFAP2A, KLF5, KRT8, SFN, DNMT1 and DNMT3A; adjusted P<0.05) and signaling pathways (Wnt, LIF, HIF-1 and AMPK signaling pathway; adjusted P<0.05) required for capturing bovine TSCs state, and reconstructed molecular trajectories of bovine placental trophoblast development. Without wishing to be bound by theory, the bovine TSC we established in this study provides a powerful model for bovine early placental establishment and early pregnancy failure.

### Example 3

Placental trophoblast cells are specialized cells in the placenta that mediate the interactions between the fetus and the mother and arise from the trophectoderm (TE) of the blastocyst. Here, we have developed a platform that allows for the derivation and long-term culture of trophoblast stem cells (TSCs) from a large animal, bovine. The resulting cell lines have TSC characteristics and can contribute to functional uninucleate and binuclear trophoblasts in vitro and in vivo. The bovine TSC established here will provide a powerful tool to study placental trophoblast differentiation and function, and early pregnancy failure. Together with embryonic stem cells, TSCs can also be used for assembly of artificial blastocysts for varies Assisted Reproductive Technology (ART) applications (e.g., in vitro breeding).

### Example 4

### Protocols/Conditions for Bovine Trophoblast Stem Cells

### Cell Culture Reagents:

| Reagent | Source | Identifier |
|---|---|---|
| N2 supplement | Gibco | 17502-048 |
| B27 supplement | Gibco | 17504-044 |
| GlutaMAX | Gibco | 35050-061 |
| AlbumiNZ ™ Bovine Albumin Low Free | MP | 02199899925 |
| Fatty Acid | biomedical | |
| MEM NEAA | Gibco | 11140-050 |
| 2-mercaptoethanol | Gibco | 21985-023 |
| Neurobasal medium | Gibco | 21103-049 |
| DME/F-12 | Cytiva | SH30023.02 |
| Recombinant Human LIF | Peprotech | 300-05 |
| CHIR-99021 (CT99021) | Sigma | SML1046 |
| (S)-(+)-Dimethindene maleate | Torcris | 1425 |
| Minocycline, Hydrochloride | Santa Cruz | sc-203339 |
| Accutase | Gibco | A11105-01 |
| ProFreeze Freezing medium | Lonza | 12-769E |
| Forskolin | Sigma | F3917 |
| Y27632 | Tocris | 1245 |
| KnockOut ™ Serum Replacement(KOSR) | Gibco | 10828028 |
| TrypLE | Gibco | 12605-010 |
| Col IV | Corning | 354233 |

| LCDM medium | Volume | |
|---|---|---|
| Neurobasal medium | 48 | mL |
| DME/F-12 | 48 | mL |
| MEM NEAA | 1 | mL |
| GlutaMAX | 1 | mL |
| N2 supplement | 0.5 | mL |
| B27 supplement | 1 | mL |
| Bovine Albumin Low Free Fatty Acid | 0.1 | g |
| 2-mercaptoethanol | 181.8 | μL |
| 10 ng/mL LIF (Stock: 200 μg/mL) | 5 | μL |
| 3 μM CHIR99021(Stock: 3 mM) | 100 | μL |
| 2 μM Dimethindene maleate (Stock: 10 mM) | 20 | μL |
| 2 μM Minocycline, Hydrochloride (Stock: 10 mM) | 20 | μL |

| Differentiation medium | Volume | |
|---|---|---|
| Neurobasal medium | 48 | mL |
| DME/F-12 | 48 | mL |
| MEM NEAA | 1 | mL |
| GlutaMAX | 1 | mL |
| N2 supplement | 0.5 | mL |
| B27 supplement | 1 | mL |
| Bovine Albumin Low Free Fatty Acid | 0.1 | g |
| 2-mercaptoethanol | 181.8 | μL |
| 2.5 μM Y27632 (Stock: 50 mM) | 5 | μL |
| 2 μM Forskolin (Stock: 10 mM) | 20 | μL |
| 4% KOSR | 4 | mL |

### Bovine IVF Embryo Production

The IVM-IVF embryos used in this study were produced using cumulus-oocyte complexes (COCs) and maintained with BO-Bioscience medium. Blastocysts were collected, and were removed the zona pellucida by pipetting, and were processed for TSC derivation.

### Derivation and Culture of Bovine TSCs

Each blastocyst was placed in a separate well of a 12-well plate that was seeded with mitomycin C-treated mouse embryonic fibroblast (MEF) cells (ATCC, Cat #: SCRC-1040).
bTSCs Thawing
1. prepare 5 mL 10% FBS in DMEM in a 15 mL tube and set the water bath to 37 °C.
2. thaw bTSC in vial in water bath and use 70% ETOH to sterilize, then wipe.
3. add entire medium of vial into 5 mL 10% FBS in DMEM.
4. centrifuge at 1000 rpm for 5 minutes.
5. use LCDM to resuspend cells and plate in 6-well plate with feeder.
bTSCs Passage
1. prepare MEF feeder one day before passage. Fresh feeder is better.
2, about 80-90% confluence, aspirate the medium and use 1 mL PBS to wash one time.
3. each well of 6-well plate is added 1 mL Accutae and put back into the incubator for 4-5 minutes. Then, use the same volume of LCDM medium to inactivate Accutase.
4. collect the cells and centrifuge at 1000 rpm for 5 minutes.
5. cells are resuspended in 1.5 mL of LCDM medium per well of 6-well plate. Normally the cells are passaged at 1:6 ratio every 6 days.
6. cells are cultured in 38.5 °C, 5% CO2 incubator.

### Changing Medium

1. warm medium at room temperature for at least 30 minutes.
2. aspirate old medium.
3. add 2 mL LCDM medium to the well and change the medium every day.
bTSCs Feeder-Free Culture
For feeder free condition, bTSCs cultured on feeder cells were passaged to Matrigel (Corning, 354234)-coated plates using MEF-conditioned-bTSC-medium (MEF-bTSC).
1. LCMD medium are incubated on MEF feeder for 24 hours, then are collected and stored at −20 °C, call it conditioned-LCDM.
2. matrigel coat plates for 1 hour at room temperature.
3. use Accutase to dissociate the cells follow passage steps.
4. aspirate Matrigel.
5. 10 μM Y-27632 is added to conditioned-LCDM, use this medium to resuspend cells and seed cells on Matrigel-coated plate. After 24 hours, remove Y-27632, and change fresh conditioned-LCDM every day.

### Cryopreserve

1. use 1 mL Accutase to dissociate the cells and 1 mL of LCDM medium to neutralize the reaction.
2. centrifuged at 1000 rpm for 5 minutes.
3. cryopreserved medium is 1:1 of (1.7 mL ProFreeze freezing medium+0.3 mL DMSO) and LCDM medium.
4. use 1 mL cryopreserved medium for each well of 6-well plate. Add the cells into cryovial.
5. place the tubes in-80 °C for 12-24h, then transfer tubes to liquid nitrogen tank.
bTSCs Differentiation
1. 2.5 μg/mL Col IV coat plate for 1 hour at room temperature.
2. 80-90% confluence bTSCs are dissociated with TrypLE for 15 minutes in incubator.
3. use the same volume of 10% FBS in DMEM to inactivate TrypLE.
4. centrifuge at 1000 rpm for 5 minutes.
5. aspirate Col IV and use PBS to rinse one time.
6. use differentiation medium to resuspend cells and seed the cells at a density of 1-1.5×10⁵ cells per well of 6-well plate.
7. the medium is changed every two days.

### Example 5

### Abstract

The human and mouse extended pluripotent stem (EPS) cells' capability to differentiate into trophoblast lineages has been debated. Here, we show that a modified chemical cocktail (LCDM: hLIF, CHIR99021, DiM and MiH) for human and mouse extended pluripotent stem (EPS) cells allows for the derivation and long-term culture of trophoblast stem cells (TSCs) from a large animal, bovine. The resulting cell lines have trophoblast stem cell characteristics and are capable of contributing to functional uninucleate and binuclear trophoblasts in vitro and in vivo. Molecular analyses of the transcriptome and epigenomics reconstruct trajectories of bovine placental trophoblast development and reveal enrichment for bovine trophoblast stem cell specific signatures. Remarkably, the LCDM condition supports long term culture of both bovine TSC and a formative ESC. The bovine TSC established in this study will provide a powerful model to study bovine early placental establishment and early pregnancy failure.

### Introduction

Placental trophoblast cells are specialized cells in the placenta that mediate the interactions between the fetus and the mother and arise from the trophectoderm (TE) of the blastocyst. In the bovine, the hatched blastocysts will enter a special stage, elongation, where trophoblast cells will differentiate and rapidly proliferate for optimal attachment to maternal caruncles. During this process, undifferentiated trophectoderm cells, or trophoblast stem cells will differentiate to uninucleate trophoblast cells and subsequently binucleate giant cells [1, 2], which drive embryo elongation and eventually fuse with uterine epithelial cells to form feta-maternal hybrid cells to establish the interface for fetus and mother [3]. Trophoblast development and function are pivotal for the success of pregnancy. Inadequate placental trophoblast development and subsequent dysfunction results in a range of adverse outcomes in conceptus/offspring, for example, the abnormities seen from bovine in vitro fertilized (IVF) or somatic cell nuclear transfer (SCNT) embryos [4-6]. A central gap in our understanding the bovine trophoblast differentiation and function is the less feasibility of in vivo experimental system or a lack of a manipulatable in vitro cell culture models that recapitulates placental cell differentiation.

To date, trophoblast stem cells (TSC) have been established from various species including mouse [7], human [8], and nonhuman primates [9]. Accordingly, in ruminants, a number of cell lines, including CT-1 and CT-5 [10], trophoblast cell line, BT-1 [11]), and more recently a undifferentiated trophectoderm cell with the support of irradiated mouse embryonic fibroblast feeders (MEFs) [12], have been derived from bovine blastocysts. However, none of these cells meet the TSC criteria, i.e., i) are able to maintain long-term self-renewal, and 2) have in vitro and in vivo developmental capacity to the functional trophoblasts. Thus, the generation of self-renewal and stable bovine TSC lines remains unexplored.

Conflict Results with EPS Conditions

One group derived EPS with in vivo embryonic and extraembryonic potency with LCDM condition [13].

Another group captured human trophoblast development with EPSCs [14].

Another group challenged human EPSCs' capability to differentiate into human trophoblast cells in vitro [15].

Still another group challenged mouse EPSCs' ability to generate trophoblast cells in vitro and in vivo [16].

In this study, we applied N2B27 basal medium supplemented with LIF, CHIR99021, Dimethindene maleate (DM), Minocycline hydrochloride (MH) to derive bovine TSCs. This medium robustly derives TSCs from bovine IVF blastocysts, and TSCs maintain long-term stable morphology, karyotype, transcriptomic and epigenomic features, and in vitro and in vivo developmental potential. The bovine TSCs we established in this study will provide a powerful model to study bovine early placental establishment and early pregnancy failure, and, without wishing to be bound by theory, can be useful for artificial reproductive technologies.

### Results

Derivation of Bovine Trophoblast Stem Cells (bTSCs) from In Vitro Produced Blastocysts

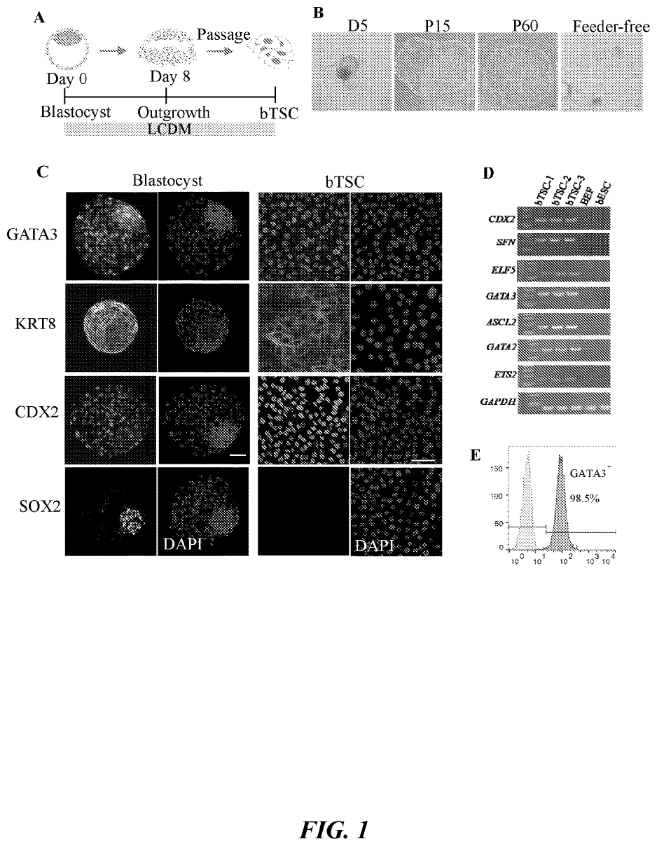

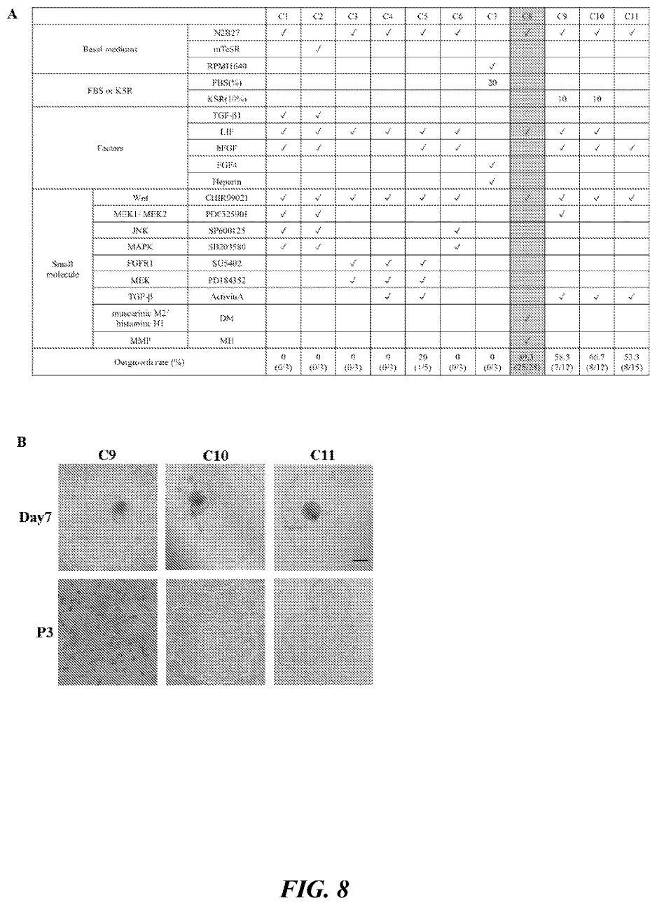

We first tested for culture conditions that allowed for robust growth bovine TE-derived cells from blastocysts (FIG. 1, panel A). We examined fourteen small molecules (eleven culture conditions) (FIG. 8, panel A) according to the important signaling pathways enriched in the primary undifferentiated trophoblast cells and the pathways involved in deriving mouse and human trophoblast stem cells and expanded potential stem cells [7, 8, 17, 18]. With the support of a layer of MEF feeder cells, four conditions (8 (C8: N2B27, LIF, and inhibitors to Wnt (CHIR99021), muscarinic M2/histamine H1 (DM), and MMP (MH); 9 (C9: N2B27, 10% KSR, LIF, bFGF, and inhibitors to Wnt (CHIR99021), MEK1/MEK2 (PD0325901), TGF-β (Activin A); 10 (C10: N2B27, 10% KSR, LIF, bFGF, and inhibitors to Wnt (CHIR99021) and TGF-β (Activin A); 11 (C11: N2B27, bFGF, and inhibitors to Wnt (CHIR99021) and TGF-β (Activin A)) can readily derive cells and form colonies from bovine blastocysts (FIG. 1, panel B and FIG. 8, panel B). However, C9 cannot support the TE derived cells beyond five passages without differentiation. The other three conditions (C8, C10, and C11) have allowed for blastocysts to attach to the feeder layers at Day 2 and form colonies at Day 7, with an outgrowth ratio of 89.3% (25/28), 66.7% (8/12) and 53.3% (8/15) for C8, C10, and C11, respectively (FIG. 8, panel A). Of note, under these three conditions, the unattached blastocysts at day 2 can be physically pressed to attach to the feeder layers and maintain outgrowth. Further maintenance of these TE derived cells showed that cells under C10 and C11 cannot survive cryopreservation, have low cell viability, and lower passage ratio (1:2). Overall, cells under C8 had a higher outgrowth rate, formed vesicles in the center of colony, self-renewed under a passage ratio of 1:6 without change of flat morphology, and maintained the normal morphology after long term culture and several rounds of cryopreservation (FIG. 1, panel B). Therefore, we refer to cells derived and maintained in C8 (LCDM: LIF, CHIR99021, DM, MH) medium as bovine trophoblast stem cells (bTSCs).

We also validated whether small molecules in C8 are required, our screening has shown that removal of any one of small molecules cannot maintain the morphology and self-renewal of bTSCs, indicating LCDM are indispensable for the derivation and maintenance of bTSC. In addition, bTSCs can be maintained under feeder free condition for long-term culture with supplementary of MEF-conditioned bTSC medium (FIG. 1, panel B).

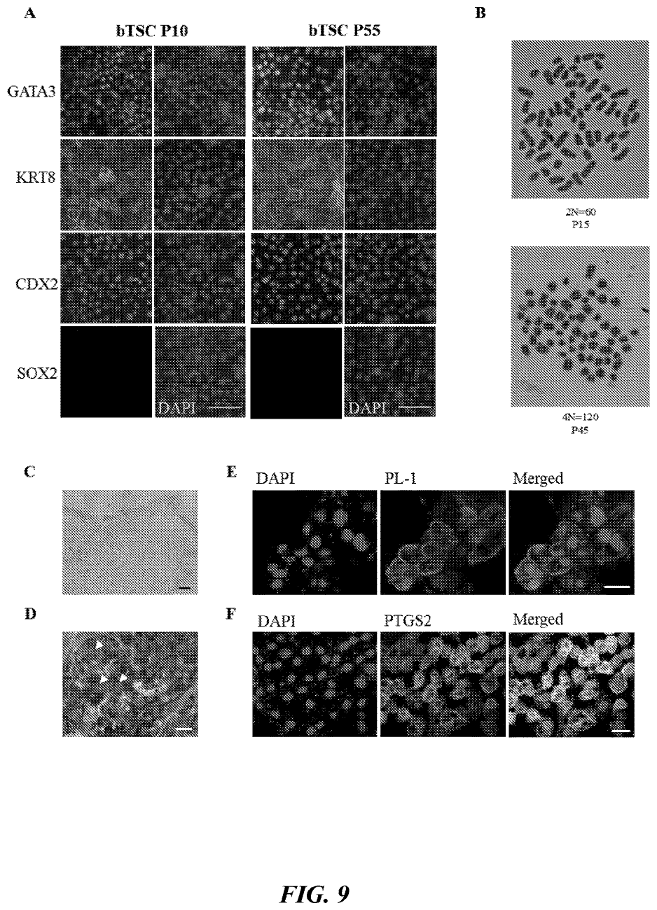

We successfully derived bTSCs and we maintained five lines for further characterization. First, bTSCs exhibited colony morphology of flattened trophoblasts and continued to proliferate for over 60 passages (FIG. 1, panel B). They had a normal karyotype during the long-term culture (FIG. 9, panel B). Second, bTSCs expressed bovine TE markers including CDX2, GATA3 and KRT8 but not the inner cell mass (ICM) marker SOX2, which had the same expression pattern with TE of blastocysts (FIG. 1, panel C and FIG. 9, panel A). Third, they expressed bovine trophectoderm associated transcriptional factors (CDX2, SFN, ELF3, GATA3, ASCL2, GATA2 and ETS2), (FIG. 1, panel D and panel E), and displayed homogeneity (FIG. 1, panel E). Collectively, these results indicate that LCDM medium supports the long-term culture of bTSCs.

Differentiation of bTSCs into Functional Trophoblast Cells

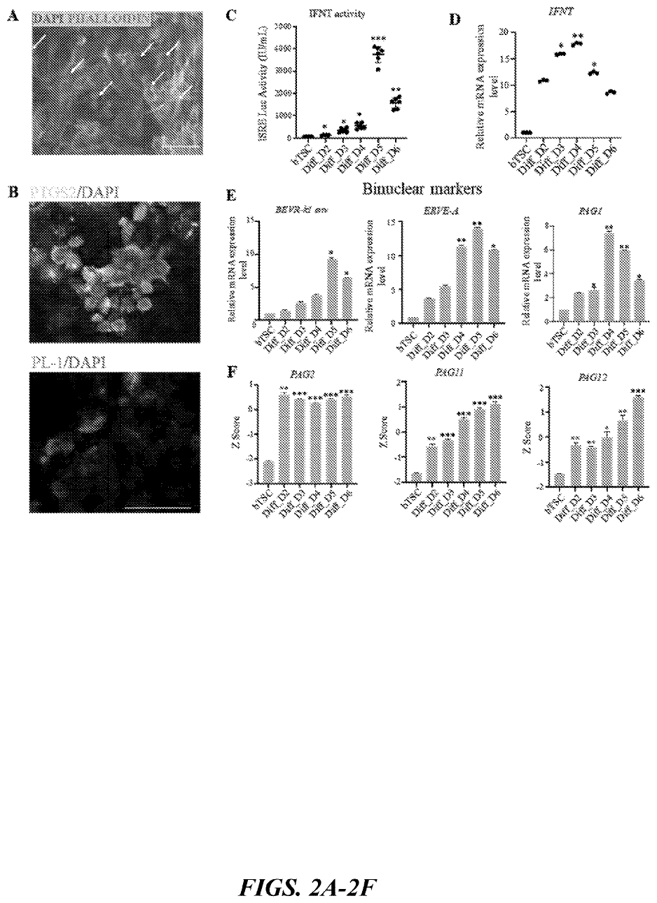

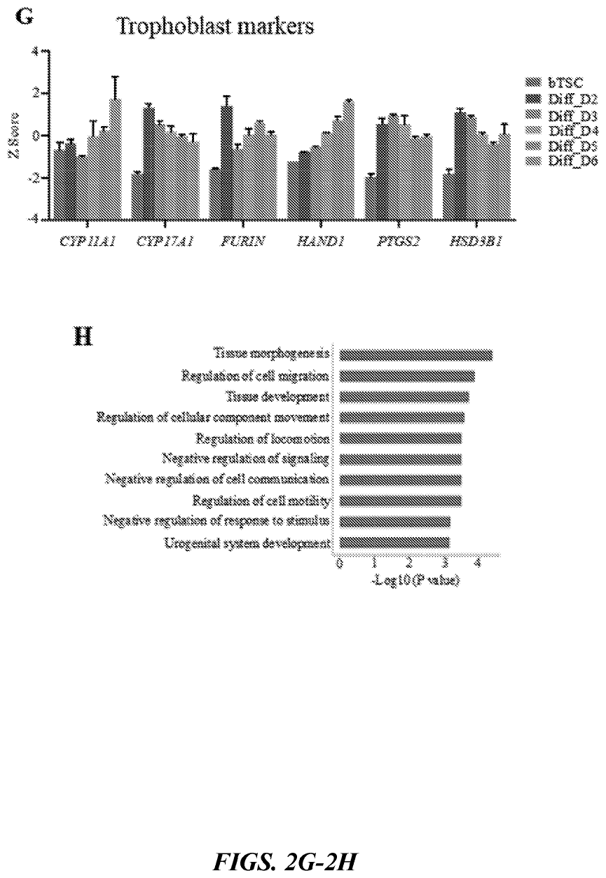

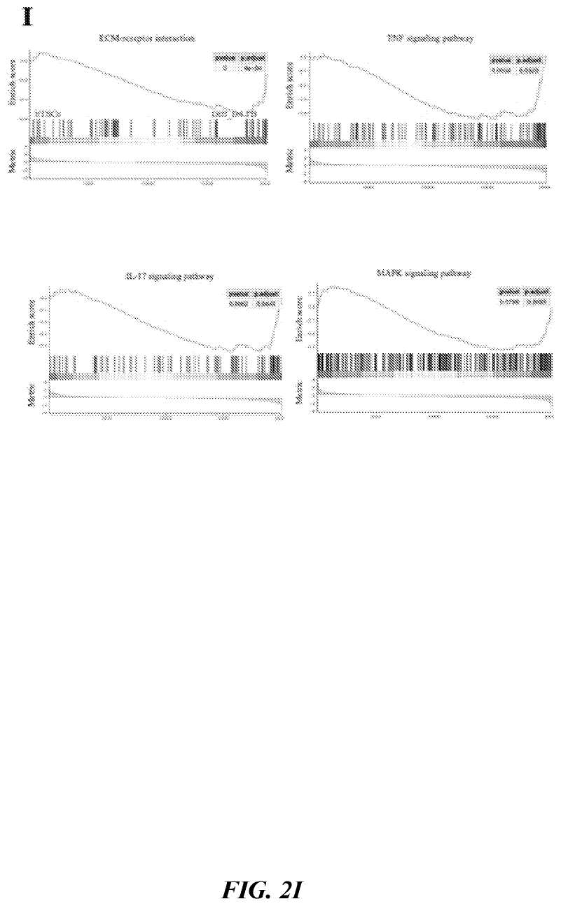

Bovine placenta consists of two cell populations, uninuclear and binuclear trophoblast cells. Binuclear trophoblast cells account for 20% of trophoblast cells throughout gestation [2]. Next, we validated whether bTSCs can differentiate into functional binuclear trophoblasts in vitro. Initial culture of bTSCs in a N2B27 basal medium cannot sustain of cell differentiation. As reported, forskolin, a cAMP agonist, can reduce lipid content and induce cell fusion [19, 20]. In a culture system containing forskolin, Y27632 and 4% KSR, bTSCs underwent differentiate into binuclear cells (FIG. 2, panel A, FIG. 9, panel C and FIG. 9, panel D). The differentiated cells expressed bovine trophoblast markers, PTGS2 and placental lactogen 1 (PL-1) (FIG. 2, panel B, FIG. 9, panel E and FIG. 9, panel F). The abundance of PTGS2 significantly increased during Day 16 to 19 compared to Day 7 to 13, which had the same pattern as IFNT2, showing a role in maternal recognition [21]. PL-1, expressed in bovine trophoblast cells, plays a vital role in placentation [22]. Interferon tau (IFNT), produced by mononuclear trophoblast cells of the conceptus in ruminants, is the signal for maternal recognition of pregnancy [23]. By testing IFNT activity using a Luciferase-based IFN stimulatory response element (ISRE) assay during differentiation [24], we found that daily release of IFNT significantly increased upon differentiation and peaked at Day 5 (FIG. 2, panel C). The mRNA expression level of IFNT was also significantly increased upon differentiation (FIG. 2, panel D). qRT-PCR analysis further showed that the expression of binuclear cell specific genes (BEVR-k1 env and bEPVE-A [25]) and pregnancy associated glycoproteins 1 (PAG1, one of the pregnancy associated glycoproteins (PAGs) that produced exclusively by the specialized trophoblastic binuclear cells of bovine placenta [26]) were significantly upregulated towards differentiation (FIG. 2, panel E). Together, these data indicate that bTSCs have the capacity contributing to functional binuclear trophoblasts.

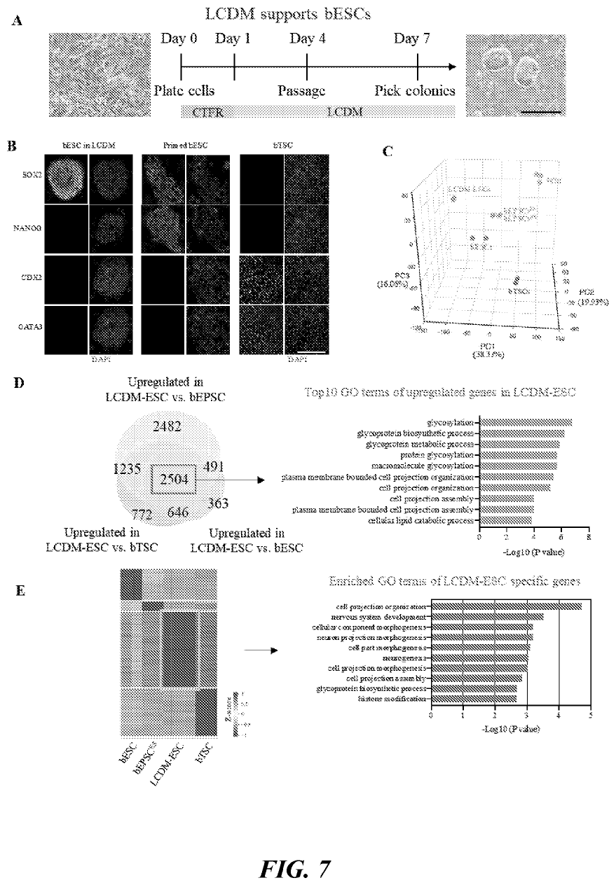

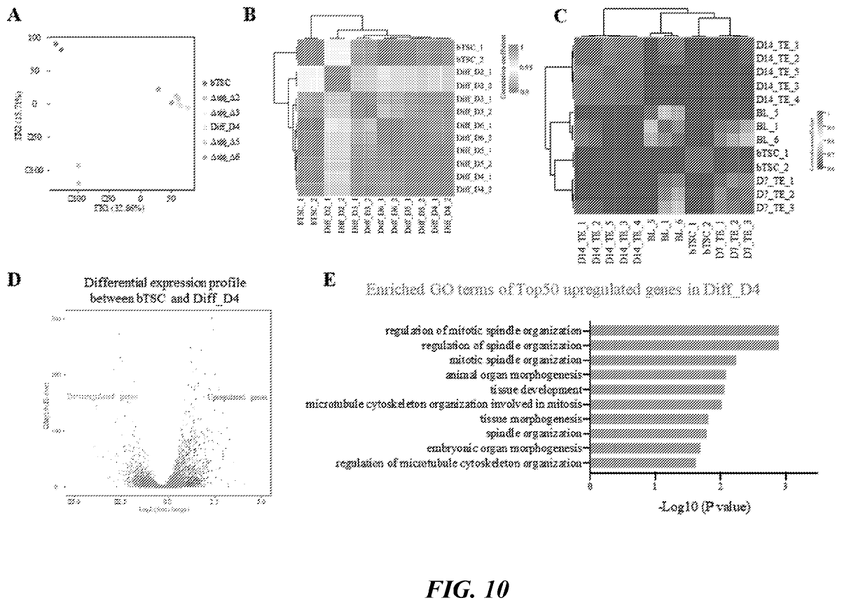

Next, we analyzed the global transcriptome over a 6-day time course of bTSC differentiation. Hierarchical clustering analysis (PCA) and Pearson correlation have confirmed that bTSCs enter into an intermediate differentiation stage on day 2, and then further differentiate into trophoblasts on day 3 to day 6 (FIG. 10, panel A and FIG. 10, panel B). RNA-seq analysis of trophoblast cell differentiation showed that PAG family genes, including PAG2, PAG11 and PAG12 exhibits the increased expression patterns during trophoblast differentiation (FIG. 2, panel F). Well known up-regulated and abundant genes in bovine placenta during pregnancy [26-28] showed elevated expression upon differentiation, including CYP11A1, CYP17A1, FURIN, HAND1, PTGS2 and HSD3B1 (FIG. 2, panel G). From PCA and Pearson correlation analysis, day 4 is most far away from bTSC (FIG. 7, panel A and FIG. 1, panel B). Annotation of the up-regulated genes in the trophoblast cells (Day 4) revealed significant enrichment of morphogenesis, cell migration, and locomotion (FIG. 2, panel H and FIG. 10, panel D). Moreover, enrichment of the top 50 differentially expressed genes in Day 4 trophoblast cells highlighted spindle organization, tissue development and morphogenesis, and microtubule cytoskeleton organization (FIG. 10, panel E). Above all confirm the presence of invasive type of trophoblast binuclear cells. In addition, in comparison to bTSCs, differentiated trophoblast cells had abundant genes involved in signaling pathways including ECM-receptor interaction, TNF, IL-17, and MAPK signaling (FIG. 2, panel I), which is consistent with the reported rise in ECM-receptor interaction, and TNF-α and IL-17 accompanying implantation and placental development in ruminants and humans, respectively [29-31].

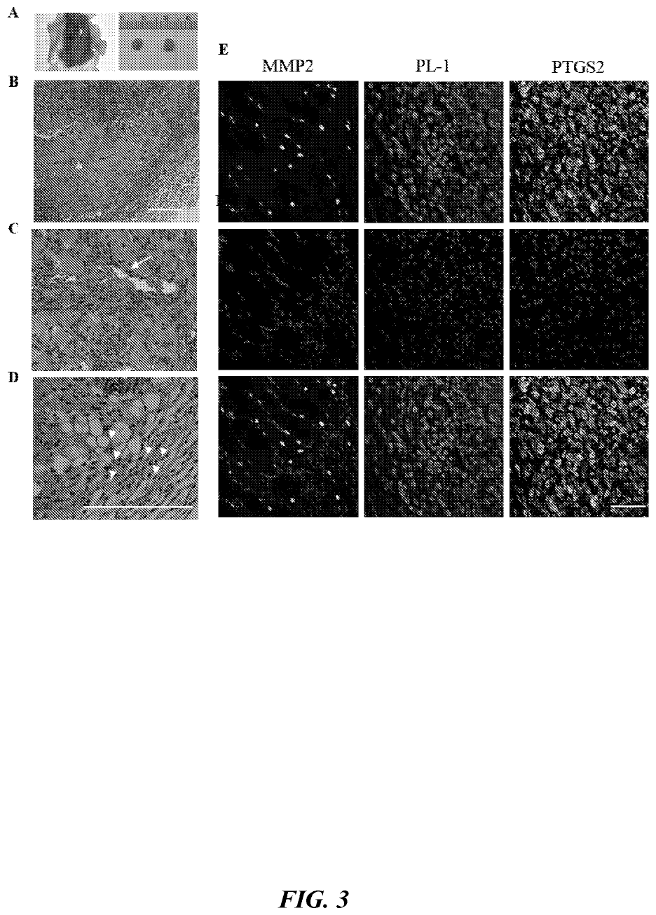

To validate the developmental potential of bTSCs in vivo, we subcutaneously injected 5×106 bTSCs into non-obese diabetic (NOD)-severe combined immunodeficiency (SCID) mice. The injected bTSCs formed ~0.5 cm lesions by day 9 (FIG. 3, panel A) and were then gradually resorbed. Immunobiological staining analysis revealed that the central area of the lesions is necrotic (FIG. 3, panel B), and the lesions contain blood-filled lacunae which mimic the nutrition exchange between fetus and mother (FIG. 3, panel C). The similar structures were also found in lesions formed by mouse and human TSCs [8]. Binuclear cells were also identified in some region of the lesions which represented typical mature trophoblast cells in bovine (FIG. 3, panel D). In addition, PL-1 positive cells and PTGS2 positive cells were observed at the peripheral region of the lesions (FIG. 3, panel E). At the margins of the lesions, we also identified MMP2 positive cells (FIG. 3, panel E). MMP2 expression increased significantly during peri-implantation stage, it is a key factor for trophoblast cells and endometrial epithelia talk and remodeling of endometrial matrices [33]. These data indicated that the bTSCs injected into NOD-SCID mice mimic some pivotal features of trophoblast cells during pregnancy.

Transcriptional Signatures of bTSCs

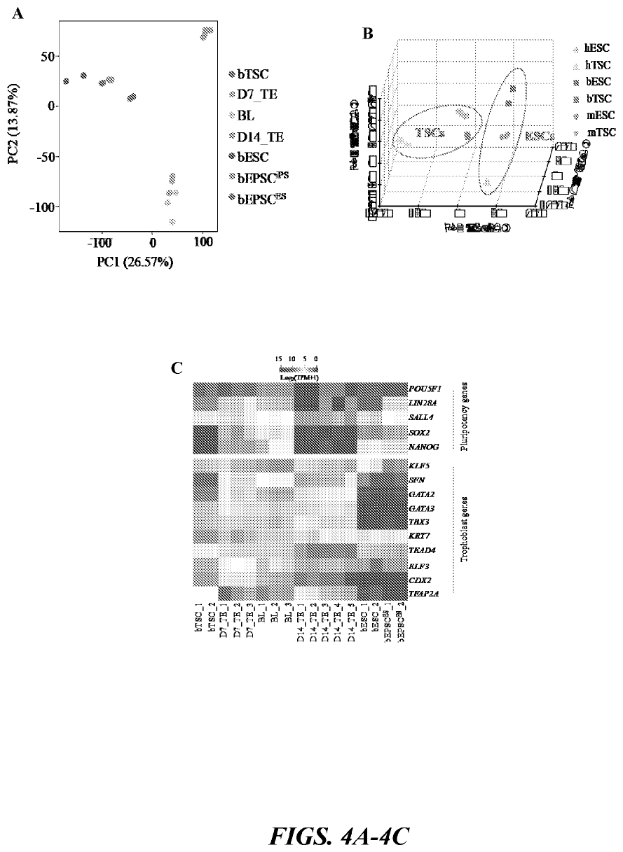

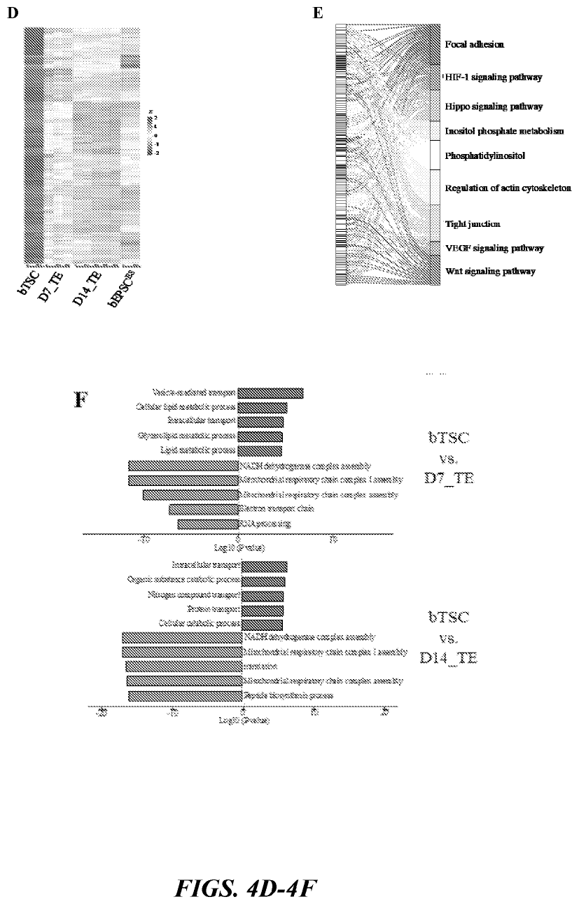

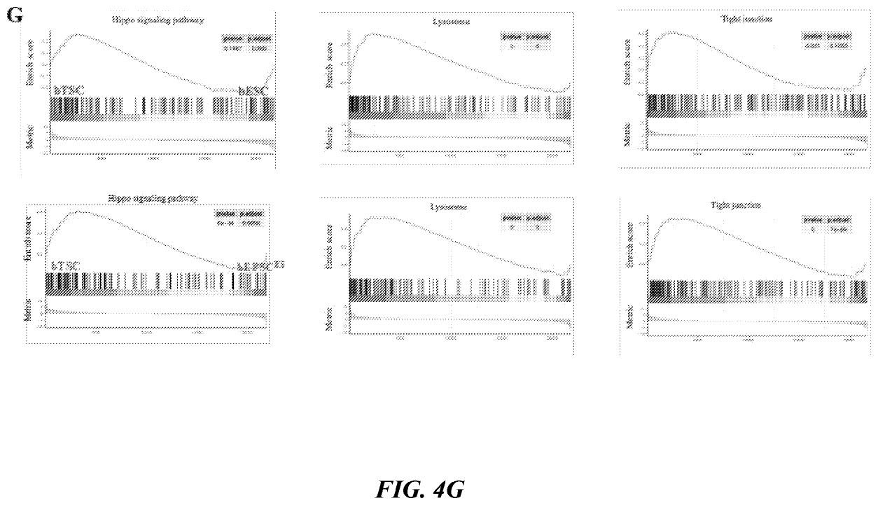

To elucidate the transcriptional features of bTSCs and whether or at which developmental stage bTSCs mimic in vivo cytotrophoblasts, we performed RNA-seq analysis of bTSCs, TE from day 7 blastocysts (D7_TE) and day 14 elongated embryos (D14_TE) in bovine, and compared our findings with those from published RNA-seq dataset of bovine primed ESCs (bESCs) and expanded potential stem cells (bEPSCs) [34, 35]. Day 7 blastocyst (BL, majority cells are trophectoderm cells) was also included. Principal Component Analysis (PCA) and Pearson correlation analysis of transcriptomic data indicated consistent measurements between biological replicates across developmental stage (FIG. 4, panel A and FIG. 10, panel C). The transcriptomic data in the PC1 dimension showed bTSCs clustered tightly as a group separate from both ESCs/EPSCs and TEs/BL (FIG. 4, panel A). The transcriptomic data in the PC2 dimension, appeared to align the bTSCs between two distinct developing trophoblast groupings, namely the stages representing trophectoderm stem cells at preimplantation embryo (D7_TE) and elongation trophoblasts at peri-implantation stage (D14_TE) (FIG. 4, panel A), demonstrating that bTSCs exist in a distinct trophoblast stem cell transition. In addition, PCA analysis of bTSCs, and TSCs/ESCs from human and mouse revealed bTSCs were clustered together with both human and mouse TSCs, but distinct from ESCs (FIG. 4, panel B), further indicating the molecular identify of trophoblast stem cells for bTSCs.

Next, we analyzed the well-known pluripotency markers and trophoblast transcriptional factors in bTSC, TE and ESCs. bTSCs expressed trophoblast markers including KLF5, SFN, GATA2, GATA3, TBX3, KRT7, TEAD4, CDX2, and TEAP2A, but not pluripotency markers (POU5F1, SOX2, and NANOG) with a few of exceptions (e.g., LIN28A and SALL4), while their expression had contrasting trends in ESCs and EPSCs (FIG. 4C). Of note, Lin28A is reported to have a functional role in regulating trophoblast differentiation and function in ruminants [36]. Differences between bTSCs and TEs was also observed, with low or no expression of trophoblast transcriptional factors (KRT7, TEAD3, ELF3, CDX2, and TFAP2A) in TEs (FIG. 4, panel C), indicating the distinct stemness of bTSC in vitro and that bTSC emerge from matured TE.

Furthermore, we identified the genes with specifically expressed in bTSCs compared to TEs and EPSCs (FIG. 4, panel D), their unique transcriptional trends indicate the molecular signatures of bTSC and their specific regulatory function in trophoblast stem cell development. KEGG pathway analysis of these transcripts uncovered key signaling that represent trophoblast cell fate including focal adhesion, HIF-1, Hippo, VEGF, and Wnt signaling pathways, actin cytoskeleton, and tight junction (FIG. 4, panel E). For example, Hippo signaling stimulates initiating TE differentiation among human, bovine and mouse [37]. Actin is one of prominent functional cytoskeletal proteins which maintain the dynamic state and vesicle transport during blastocoel formation, blastocyst hatching and embryo implantation [12]. Trophoblast cells are connected by tight junctions to prevent the exchange of fluid and allow the accumulation of fluid inside of blastocyst. Focal adhesion are upregulated in placental development in goat and sheep [29], dysregulated focal adhesion proteins can affect binuclear organization and trophoblast polarity [38].

Finally, we performed pairwise comparisons of bTSC transcriptome with those of D7_TE, D14_TE, bESCs, and bEPSCs. GO analysis of up-regulated genes in bTSCs compared to D7_TE and D14_TE revealed a significant enrichment of intracellular transport and metabolic process, while GO enriched terms of down-regulated genes involved in mitochondrial function (FIG. 4, panel F). The significantly up-regulated pathways in bTSCs compared to bESCs and bEPSCs included Hippo signaling pathway, lysosome and tight junction (FIG. 4, panel G). These specific enriched signaling and metabolism machinery represent another unique feature of bTSCs.

Together, these data indicate that bTSCs represent a unique stem state of trophoblast fate and resemble matured TE in terms of transcriptome characteristics.

Epigenomic Features of bTSCs

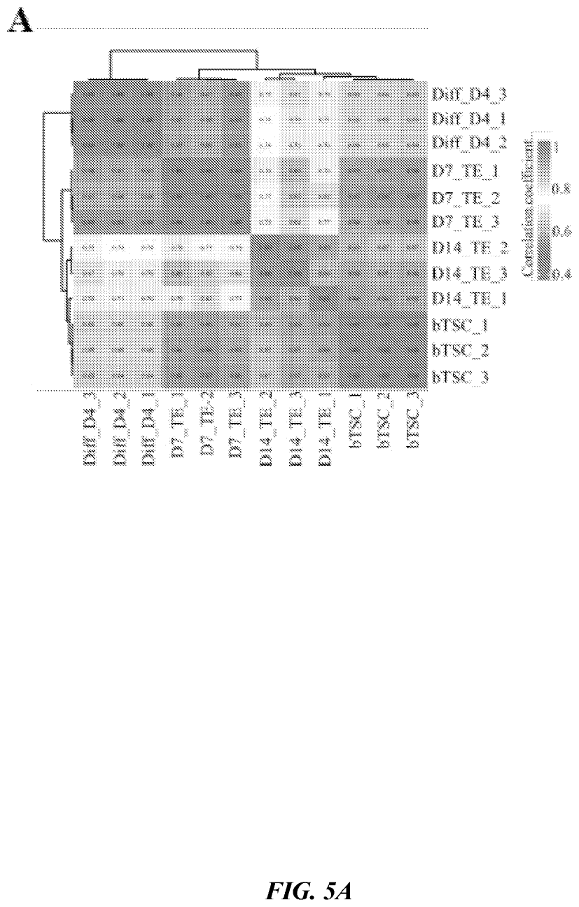

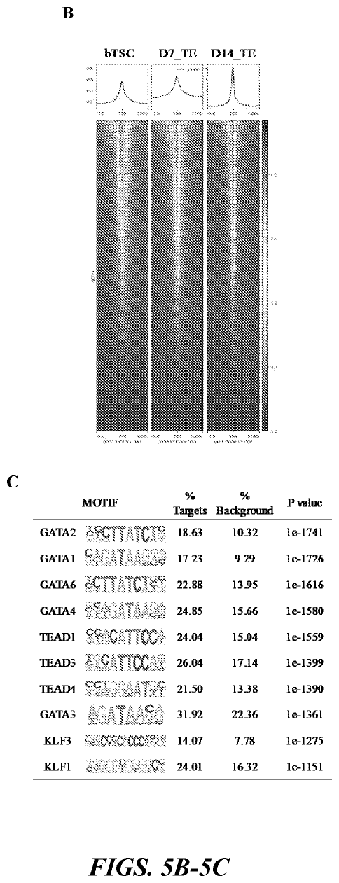

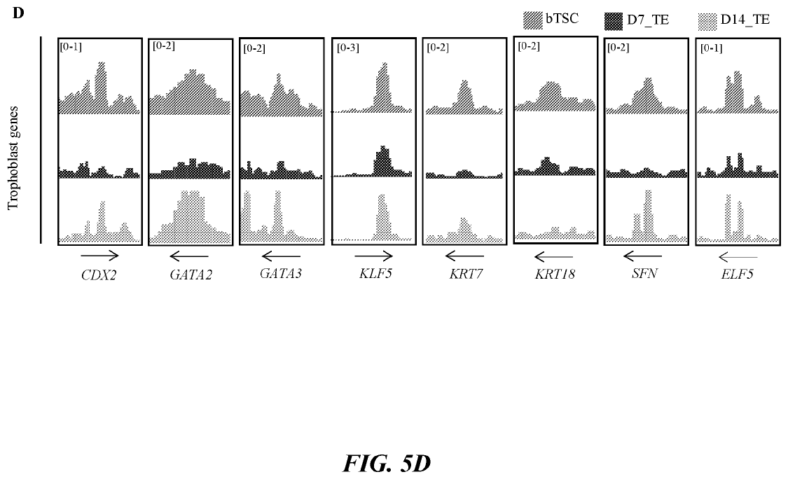

To gain insight into the epigenetic regulation of the bovine trophoblast program, we performed ATAC-seq and WGBS analysis of bTSCs, D7_TE, and D14_TE. ATAC-seq analysis revealed that bTSCs have shown very similar profiles of global chromatin accessibility with those of D7_TE and D14_TE (FIG. 5, panel A and panel B), while distinct with those of differentiated trophoblasts (FIG. 5, panel A). We found the binding motifs of trophectoderm lineage markers including GATA1, 2, 3, 4, 6, TEAD 1, 3, 4, and KLF1 and 3 are top ten strongly enriched in bTSCs (FIG. 5, panel C). By mining the chromatin accessibilities of trophoblast marker genes, we found bTSCs showed more similarity with D14_TE compared to D7_TE (FIG. 5, panel D). Furthermore, we analyzed the differential enrichment of ATAC-seq peaks between bTSCs, D7_TE and D14_TE. Genes with open chromatin accessibilities in bTSCs compared to D7_TE and D14_TE were involved in MAPK, HIF-1, TGF-beta, focal adhesion, and signaling regulating PSCs (FIG. 5, panel E and panel F), which is in agreement with our findings of transcriptome analysis. The genes with lower accessible chromatin in bTSCs compared to D7_TE represented the pathways including cGMP-PKG, Hippo, and calcium signaling pathways, arginine and proline metabolism and cellular senescence, while genes involved in Rap1, oxytocin, apelin, Estrogen, Wnt, and GnRH signaling pathways had more accessible chromatin in D14_TE compared to bTSCs (FIG. 5, panel E and panel F). This analysis identified important candidate regulators and signaling networks directing bovine trophoblast lineage specification.

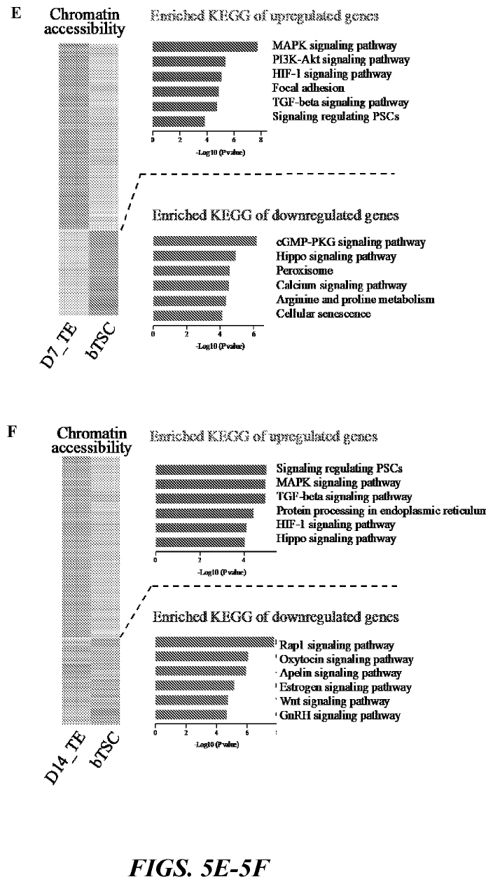

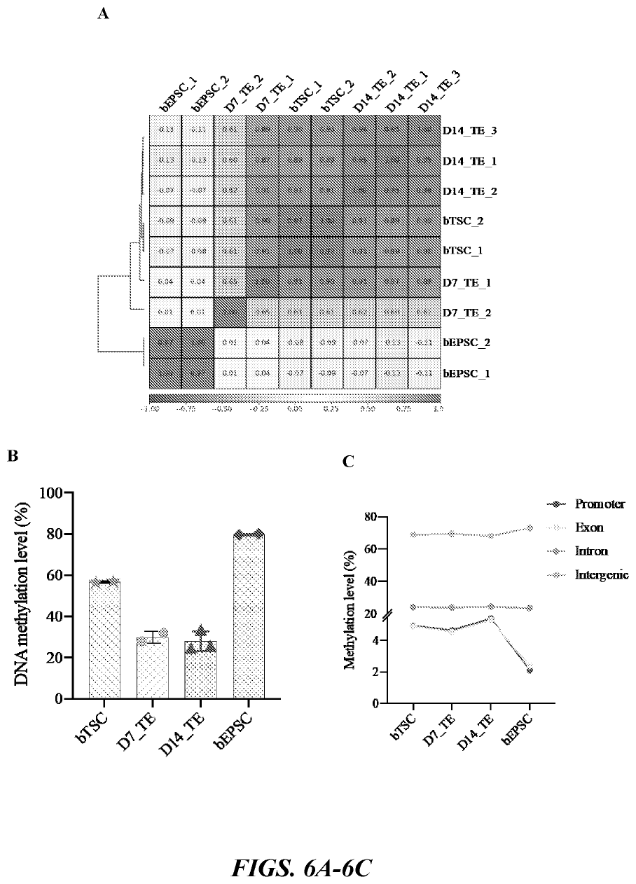

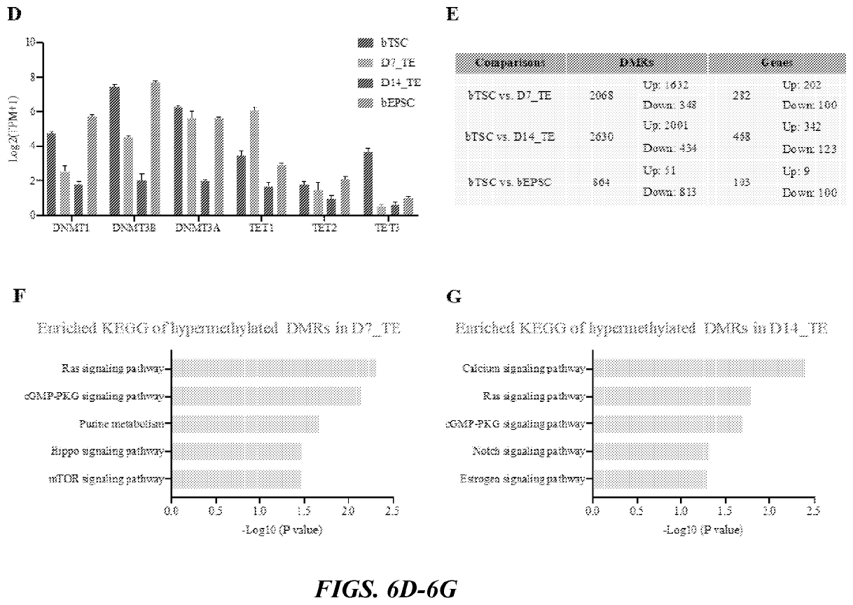

Comparing WGBS data of bTSCs, D7_TE and D14_TE with those of bEPSCs (FIG. 6, panel A), we found that DNA methylomes of bTSCs were closer to those of D7_TE and D14_TE, but significantly distinct with those of bEPSCs. The average methylation levels of bTSCs (56.75%) were substantially higher than that of D7_TE (29.90%) and D14 TE (28.03%), but lower than that of bEPSCs (79.80%) (FIG. 6, panel B). Also, we found that promoter and exon regions were consistently lowly methylated in bTSCs, D7_TE, D14_TE and bEPSCs, and much lower in bEPSCs, while the methylation levels of intron and intergenic regions were high (FIG. 6, panel C). These results indicate that the global methylation levels mainly reflect those of noncoding regions. By analyzing gene expression of DNA methyltransferases (DNMT1, DNMT3A and DNMT3B) and DNA methylcytosine dioxygenase (TET1, TET2 and TET3) according to RNA-seq data, we found that expression levels of DNMT1, DNMT3A and DNMT3B were high in bTSCs and bEPSCs, in line with high DNA methylation levels in bTSCs and bEPSCs (FIG. 6, panel D). We identified differentially methylated regions (DMRs) between bTSCs and D7_TE (2068), D14_TE (2630) and bEPSCs (864), respectively (FIG. 6E). Hypermethylated DMRs in D7_TE compared to bTSCs enriched in Ras signaling, cGMP-PKG signaling, Hippo signal and mTOR signaling (FIG. 6, panel F). While hypermethylated DMRs in D14_TE mainly focused on calcium signaling, Ras signaling, cGMP-PKG signaling, Notch signaling and estrogen signaling (FIG. 6, panel G). These results are consistent with those of transcriptome and chromatin accessibility. Taken together, these signaling are vital players in regulating trophoblast development.

### LCDM Condition Supports the Bovine ESCs

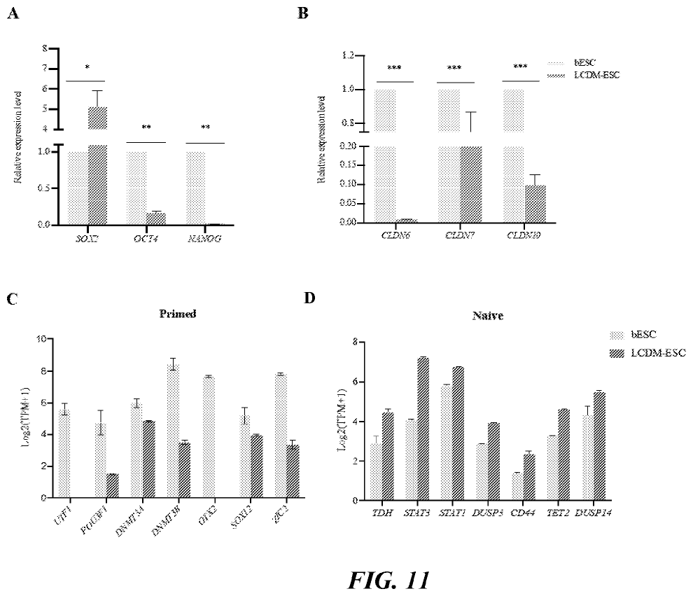

LCDM was previously used to generate mouse EPSC [39], we next sought to test if LCDM can sustain bovine ESCs. Under the LCDM condition, bovine primed ESCs maintained stable growth kinetics and exhibited colony morphology of dome shaped naïve-like ESCs during long-term self-renewal (FIG. 7, panel A). The LCDM-ESCs had an increased expression of SOX2 compared to primed bESCs but were negative for NANOG (FIG. 7, panel B). The mRNA expression level of SOX2 was significantly increased comparing with primed bESCs, while the expression levels of OCT4 and NANOG were significantly decreased (FIG. 11, panel A). The LCDM-ESCs were confirmed no expression of trophoblast markers CDX2 and GATA3, which is distinct from of TSCs cultured in the same condition (FIG. 7, panel B). In human, primed ESCs form tight junctions and express high levels of tight-junction related genes when compared to naïve ESCs [40]. The mRNA expression levels of some tight-junction related genes (CLDN6, CLDN7 and CLDN10) in LCDM-ESCs were significantly decreased when compared with primed ESCs (FIG. 11, panel B). We then performed RNA-seq analysis of LCDM-ESCs and compared to transcriptomes of bESCs, bEPSCs, ICM and bTSCs. PCA analysis showed LCDM-ESCs as a separate group and placed LCDM-ESCs between ICM and bESCs, and more closed to bEPSCs (FIG. 7C), indicating LCDM-ESCs can be a specific embryonic stem cell type between primed and naïve ESCs. To validate the pluripotency state of LCDM-ESCs, we analyzed the expression of some naïve and primed pluripotency markers used in mouse and human [35, 40, 41]. Compared with primed ESCs, LCDM-ESCs expressed higher levels of naïve pluripotency and lower levels of primed pluripotency genes (FIG. 11, panel C and panel D). We identified 2504 genes upregulated in three pairwise groups which were enriched in GO terms including glycosylation, cell projection organization, cell projection assembly and cellular lipid catabolic process (FIG. 7, panel D). When performing GO analysis for genes only specifically upregulated in LCDM-ESCs, we found that these genes were mainly related to cell projection organization, nervous system development, cell part morphogenesis and histone modification (FIG. 7, panel D). These results demonstrate that LCDM-ESCs reside in a pluripotency state distinct from primed ESCs and EPSCs.

### Discussion

In this study, we found LCDM can efficiently establish bovine TSCs from in vitro blastocysts. Bovine TSCs in our system had the capacity to indefinitely self-renew, maintained a normal karyotype in long-term culture, and possessed the potential to differentiate into functional uninucleate and binuclear trophoblasts. In addition to above, the transcriptome and epigenome analysis indicate that bovine TSCs harbored some properties unique to mature trophoblast cells. Furthermore, TSCs injected into NOD-SCID mice mimicked some important processes of trophoblast cells during elongation and implantation.

The transcription factors for bovine TSCs are different from mouse and human TSCs. We found CDX2 expression was high (>209 TPM) in bovine TSCs but much lower in trophoblast cells, and even was undetectable in bESCs and bEPSCs (0 TPM), indicating CDX2 is a vital regulator in bovine TSCs. It's consistent with mouse TSCs, which Cdx2 is required for self-renewal [42]. However, CDX2 expression was very low in human TSCs and cytotrophoblast cells, it is dispensable for proliferation of human TSCs [8]. More strikingly, SOX2 was undetectable in bovine TSCs (0 TPM), while was much higher in bESCs (around 400 TPM) and bEPSCs (>220 TPM), showing SOX2 is dispensable for bovine TSCs maintenance. This notion is supported by expression of SOX2 in bovine starts at the 16-cell stage and then restricts to the ICM of blastocysts [43]. In mouse TSCs, Sox2 can support FGF-independent self-renewal of TSCs [44], also can combine with Tfap2c regulate TSC specific genes [42].

We found that LIF, activator of Wnt (CHIR99021) and inhibitors of PARP or MMP (MiH) and muscarinic and histamine H1 receptors (DiM) are important for derivation and maintenance of bovine TSCs. And transient inhibition of Rho-associated protein kinase (ROCK) is beneficial for passage survival. For human TSCs, activation of Wnt and EGF and inhibition of TFG-β, histone deacetylase (HDAC) and ROCK are important [8], while activating FGF and TGF-β and inhibiting Wnt and ROCK are needed when deriving mouse TSCs [45]. Only Wnt signaling is conservative in human and bovine. There are marked differences in placenta development among mouse, human and bovine. In mouse, the polar TE generates extra-embryonic ectoderm (ExE) and ectoplacental cone (EPC), and there are four different trophoblast cells with their own roles, establishing the exchange surface, lining implantation site, contacting with maternal decidua [46]. In human, primitive syncytiotrophoblast cells finish the initial invasion, migration of mononuclear cytotrophoblast cells form the primary villi [46]. But for bovine, there is a huge difference after blastocyst stage. The embryos will hold the implantation and enter elongation on day 13, morphology of embryos finally transit into filamentous, TE length and weight increase a lot, the length of conceptus can be over 20 cm after elongation [47]. Therefore, it is reasonable mouse, human and bovine require different pathways to support TSC self-renewal.

PARP1 plays diverse roles, such as DNA damage, chromatin modification, transcription and so on [48]. Parp 1 is involved in extraembryonic developmental potency in mouse EPSCs [39], and PARP activity can be detected in bovine placenta [49], but the exact functions still need more work. MiH is also a non-selective inhibitor of MMP. MMP2 and MMP9 are expressed in cohort of tissues in bovine placentome [50], their activity plays a vital role during implantation in cows. When MMP is inhibited, bovine TSC will keep self-renewal, rather than differentiate into binuclear or multinuclear cells. When comparing with differentiated cells, the down-regulated of MAPK signaling was observed in bovine TSCs (FIG. 2, panel I). MAPK signaling is one of the important downstream signaling of muscarinic and histamine receptor [51].

Differentiated TSCs lost their proliferation capacity, but some differentiated cells had two nuclei. IFNT, the signal for pregnancy recognition, the activity was detected in the medium and the expression level was significantly increased during differentiating. PTGS2, regulated by IFNT during early pregnancy, was detected in both transcription and protein level. However, little is known about the molecular mechanisms controlling the balance between uninucleate and binuclear trophoblast cells, which decides the success of elongation. And more precise markers at different pregnancy stages are needed to identify to support pregnancy test.

Further, we found that LCDM can support bovine ESCs growth. Under LCDM, bovine primed ESCs change into tighter and domed-like colonies, which expression of SOX2 is significantly increased. Moreover, LCDM-ESCs have higher expression levels of naïve markers and lower levels of primed markers compared to primed bESCs (FIG. 11, panel C and panel D). Our results indicate that LCDM-ESCs have several distinct cellular and molecular features with primed bESCs and bEPSCs. Recently, Smith proposed that there is a specific stage, called formative phase, existed between naïve and primed pluripotency phased [52]. There are some criteria for evaluating formative pluripotency. The important rules are that formative cells have the competence for forming chimaeras and germ cells.

In conclusion, we have established bovine TSCs from IVF blastocysts, which opens new avenues for studying molecular and functional mechanisms of bovine trophoblast cells. Furthermore, our bovine TSC provides new possibilities for understanding the pathogenesis of failure embryo development associated with trophoblast defects.

### Materials and Methods

### Bovine IVF Embryo Production

The IVM-IVF embryos used in this study were produced as previously described [53]. Briefly, bovine cumulus-oocyte complexes (COCs) were aspirated from selected follicles of slaughterhouse ovaries. BO-IVM medium (IVF Bioscience) was used for oocyte in vitro maturation, after which IVF was performed using cryopreserved semen from a Holstein bull with proven fertility. Embryos were then washed and cultured in BO-IVC medium (IVF Bioscience) at 38.5 °C with 6% CO2. Blastocysts were collected, and were removed the zona pellucida by pipetting, and were processed for TSC derivation.

Mouse trophoblast stem cells (mTSCs) was derived from blastocysts in the presence of fibroblast growth factor 4 (FGF4) [7]. mTSCs are the best in vitro model to study mouse trophoblast cells in molecular and functional analysis. Recently, human trophoblast stem cells (hTSCs) have been established from blastocysts and cytotrophoblast (CT) cells by activating WNT and EGF and inhibiting TFG-β, histone deacetylase (HDAC) and Rho-associated protein kinase (ROCK) [8]. hTSCs have the capacity to give rise to CT, extravillous CT (EVT) and syncytiotrophoblast (ST) in vitro. For mouse, Cdx2, Gata3, Eomes and Elf5 are essential to maintain the undifferentiated state of mTSCs [42], while for human, TP63, GATA3 and TEAD4 are reported as important players for the undifferentiated state [8].

### Derivation and Culture of Bovine TSCs

Each blastocyst was placed in a separate well of a 12-well plate that was seeded with mitomycin C-treated mouse embryonic fibroblast (MEF) cells. The embryos were cultured in bovine TSC medium containing DMEM: F12 (Gibco) and Neurobasal medium (Gibco) (1:1), 0.5× N2-supplement (Gibco), 0.5× B27-supplement (Gibco), 1×NEAA (Gibco), 1× GlutaMAX (Gibco), 0.1 mM 2-mercaptoethanol (Gibco), 0.1% BSA (MP biomedicals), 10 ng/mL LIF (Peprotech, 300-05), 3 μM CHIR99021 (Sigma, SML1046), 2 μM Dimethindene maleate (DM) (Tocris, 1425) and 2 μM Minocycline hydrochloride (MH) (Santa cruz, sc-203339). The cells were incubated at 38.5 °C and 5% CO2. After 48 hours of plating, the unattached embryos were pressed against to the bottom of the plates with needles under microscope. The culture medium was changed daily. At day 7 or 8, outgrowths were dissociated by Dispase (STEMCELL Technologies) for 5-10 mins at 38.5 °C, followed by twice washes with DMEM/F12. bTSC were passaged mechanically under a microscope. For optimal survival rate, 10 μM Rho-associated protein kinase (ROCK) inhibitor Y-27632 (Tocris, 1254) was added to the culture medium for 24 hours.

Once established, bTSCs were passaged every 6 days at a 1:6 split ratio using Accutase (Gibco, A1110501). Each well of bTSCs was dissociated by 1 mL Accutase for 5 mins at 38.5 °C, the same volume of bTSCs medium was used to dilute Accutase for neutralizing the reaction. bTSCs were cryopreserved by ProFreeze Freezing medium (Lonza, 12-769E) according to the manufacturer's instructions.

For feeder free condition, bTSCs cultured on feeder cells were passaged to Matrigel (Corning, 354234)-coated plates using MEF-conditioned-bTSC-medium (MEF-bTSC).

### Differentiation of Bovine TSCs

Bovine TSCs were grown to 80-90% confluence in the bTSCs medium and dissociated with TrypLE (Gibco, 12605-010) for 15 min at 38.5 °C. Then, bTSCs were seeded in a 6-well plate which was coated with 2.5 μg/mL Col IV (Corning, 354233) at a density of 1-1.5×105 cells per well and cultured in 2 mL differentiation medium containing DMEM: F12 and Neurobasal medium (1:1), with 0.5× N2-supplement, 0.5× B27-supplement, 1× NEAA, 1× GlutaMAX, 0.1 mM 2-mercaptoethanol, 0.1% BSA, 2.5 μM Y27632, 2 μM Forskolin (Sigma, F3917) and 4% KSR (Invitrogen, 10828028). The medium was changed every two days.

### Culture of Bovine Primed ESCs

Bovine primed ESCs (bESCs) were maintained on mitomycin C-treated MEF in mTeSR1 (STEMCELL Technologies, 85851) supplemented with 2.5 μM IWR1 (Sigma, 10161) and 20 ng/mL FGF2 (Perotech, 100-18B). bESCs were passaged every 4 days at a 1:3 ratio using TrypLE (Gibco, 12605-010), fresh medium was changed every day. Cells were incubated at 37 °C and 5% CO2.

### LCDM Culture bESCs

24 hours after passaging bESCs, the medium was changed to LCDM. At day 4, medium was aspirated and 1 mL 0.5 mM EDTA (Invitrogen, 15575020) was added to each well, then incubated at 37 °C for 4-5 min. Aspirated EDTA and washed the wells with PBS twice. LCDM was added to collect cells by pipetting. The cells were centrifuged at 1000 rpm for 5 min. LCDM plus 10 μM Y27632 was used to resuspend the cell pellet. The cells were plated on mitomycin C-treated MEF. At day 7, domed colonies appeared and enlarged. These colonies were picked for purifying and expanding. The purified LCDM-ESCs were passaged every 3 days at a ratio of 1:4.

### Immunofluorescence Staining

Cells or blastocysts were fixed in 4% paraformaldehyde (PFA) for 20 min at room temperature, and then rinsed in wash buffer (0.1% Triton X-100 and 0.1% polyvinyl pyrrolidone in PBS) for three times. Following fixation, cells were permeabilized with 1% Triton X-100 in PBS for 30 min and then rinsed with wash buffer. Cells were then transferred to blocking buffer (0.1% Triton X-100, 1% BSA and 0.1 M glycine) for 2 hours at room temperature. Subsequently, the cells were incubated with the primary antibodies overnight at 4 °C. The primary antibodies used in this experiment include anti-SOX2 (Biogenex, an833), anti-CDX2 (Biogenex, MU392A; 1:200), anti-GATA3 (Cellsignaling, D13C9; 1:200), and anti-KRT8 (Origene, BP5075; 1:300). For secondary antibody incubation, the cells were incubated with Fluor 488- or 555- or 647-conjugated secondary antibodies 1 hour at room temperature. ProLong Diamond Antifade (DAPI included) was used to stain nuclei. The images were taken with a fluorescence confocal microscope (Leica).

Paraffin sections were deparaffinized and then boiled in sodium citrate buffer (pH 6.0) for 20 min for antigen retrieval. Sections were blocked in 5% goat serum in TBST for 1 hour and incubated with primary antibodies at 4 °C overnight. Then, the sections were incubated with fluorescence-conjugated secondary antibodies for one hour at room temperature. Nuclei were stained with DAPI (Invitrogen, D1306).

### Quantitative Real-Time PCR

Total RNA was extracted from cells using RNeasy Micro Kit (Qiagen) according to the manufacture's protocol. First-strand cDNA was synthesized using the iScript cDNA Synthesis Kit (BIO-RAD). The qRT-PCR was performed using SYBR Green PCR Master Mix (BIO-RAD) WITH specific primers (Table S1). Data were analyzed using the BIO-RAD software provided with the instrument. The relative gene expression values were calculated using the ΔΔCT method and normalized to internal control GAPDH.

### IFNT Activity Analysis

IFNT activity was measured by a established IFN stimulatory response element-reporter assay [24]. Briefly, 5-10×105 Madin-Darby bovine kidney cells (MDBK) that are stably transduced with an ISRE-Luc reporter were plated into a well of 96-well polystyrene plates with opaque walls and optically clear bottoms (Corning) and cultured in MDBK growth medium (high glucose DMEM, 10% FBS and 1% Pen/Strep) at 37 °C for 4 hours. After removal of MDBK growth medium, 50 μL of sample or standard (Recombinant human IFN-«, IFNA: Millipore, IF007) were added. The standard curve was generated by a 1:3 serial dilution of IFNA. Cells were incubated at 37 °C for 16 hours, then 50 μL One-Glow Luciferase reagent (Promega Corp; E6120) were added into each well, with a final volume of 100 μL. After mixture at a shaker platform for 10 minutes, the measurement was performed in a plate reader.

### TSCs Lesion Assay

bTSCs cells were grown to about 80% confluence in the bTSCs medium and dissociated with TrypLE. 5×106 bovine TS cells were resuspended in 200 μL 1:1 of bTSC medium and Matrigel, and subcutaneously injected into 6-month-old non-obese diabetic (NOD)-severe combined immunodeficiency (SCID) mice. Lesions were collected at day 7 and 9, fixed in 4% PFA overnight at 4 °C for analysis.

### Karyotyping Assay

bTSCs were incubated with bTSC medium containing 1 mL KaryoMAX colcemid solution (Gibco, 15212012) at 38.5 °C for 4-5 hours. Cells were then dissociated using 1 mL Trypsin (Gibco, 25200-056) at 38.5 °C and centrifuged at 300×g for 5 min. The cells were resuspended in 1 mL PBS solution and centrifuged at 400×g for 2 min. The supernatant was aspirated and 500 μL 0.56% KCl was added to resuspend the cells. The cells were incubated for 15 min, then centrifuged at 400×g for 2 min. 1 mL cold fresh Carnoy's fixative (3:1 methanol: acetic acid) was added to resuspend the cells, followed by a 10 min incubation on ice. After centrifuge, 200 μL Carnoy's fixative was added to resuspend the cells. Cells were dropped on the clean slides and air dried, and soaked in a solution (1:25 of Giemsa stain (Sigma, GS500): deionized water) for 9 min. Slides were rinsed with deionized water and air dried. The images were taken by Leica DM6B at 1000× magnification under oil immersion.

### RNA-Seq Analysis

Total RNA of bovine TSCs and ESCs was extracted using RNeasy Micro Kit (Qiagen). Pure trophectoderm of day 7 were isolated by placing embryos in a Petri dish with phosphate-buffered saline and performing microsurgery using a microblade under a microscope. The RNA-seq libraries were generated by using the Smart-seq2 v4 kit with minor modification from manufacturer's instructions. Briefly, mRNA was captured and amplified with the Smart-seq2 v4 kit (Clontech). After AMPure XP beads purification, amplified RNAs were quality checked by using Agilent High Sensitivity D5000 kit (Agilent Technologies). High-quality amplified RNAs were subject to library preparation (Nextera XT DNA Library Preparation Kit; Illumina) and multiplexed by Nextera XT Indexes (Illumina). After purification of library with AMPure XP beads (Beckman Coulter), the concentration of sequencing libraries was determined by using Qubit dsDNA HS Assay Kit (Life Technologies). The size of sequencing libraries was determined by means of High Sensitivity D5000 Assay in at Tapestation 4200 system (Agilent). Pooled indexed libraries were then sequenced on the Illumina NovoSeq platform with 150-bp paired-end reads.

The Salmon tool was applied to quantify the gene expression profile from the raw sequencing data, by using the Ensembl bovine genome annotation (ARS-UCD1.2). Transcript per million reads (TPM) was used as the unit of gene expression. The edgeR tool [55] was applied to identify differentially expressed genes. The TMM algorithm implemented in the edgeR package was used to perform normalization of the read counts and estimation of the effective library sizes. Differential expression analysis was performed by the likelihood ratio test implemented in the edgeR package. The conventional statistical analyses were performed based on the R platform. The “cor.test” function was used to perform Spearman's rank correlation test. Principal component analysis (PCA) on the gene expression profile was performed by using the “dudi.pca” function within the package “ade4”. The heatmaps were plotted by the “heatmap.2” function within the package “gplots”. The gene ontology and pathway analysis were performed by means of the David tool [56].

In total, we sequenced two replicates of bTSCs, trophoblasts differentiated at day 2, 3, 4, 5 and 6, three replicates of whole blastocysts and day 7 trophectoderm cells selected from the same batch used for bTSCs derivation, and three replicates of bovine ESCs cultured in LCDM condition. The RNA-seq datasets of bovine day 14 trophectoderm [57], ESCs [35] and EPSCs [34] were downloaded from previous publications, respectively.

### ATAC-Seq Analysis

The ATAC-seq libraries from fresh cells were prepared as previously described [53]. Shortly, cells or embryos were lysed on ice, then incubated with the Tn5 transposase (TDE1, Illumina) and tagmentation buffer. Tagmentated DNA was purified using MinElute Reaction Cleanup Kit (Qiagen). The ATAC-seq libraries were amplified by Illumina TrueSeq primers and multiplexed by index primers. Finally, high quality indexed libraries were then pooled together and sequenced on Illumina NovoSeq platform with 150-bp paired-end reads.

The ATACseq analysis was followed our established analysis pipeline [53]. Quality assessed ATAC-seq reads were aligned to the bovine reference genome using Bowtie 2.3 with following options: -very-sensitive -X 2000 -no-mixed -no-discordant. Alignments resulted from PCR duplicates or locations in mitochondria were excluded. Only unique alignments within each sample were retained for subsequent analysis. ATAC-seq peaks were called separately for each sample by MACS2 with following options: -keep-dup all -nolambda -nomodel. The ATAC-seq bigwig files were generated using bamcoverage from deeptools. The ATAC-seq signals were visualised in the Integrative Genome Viewer genome browser. The annotations of genomic features, including transcription start sites, transcription end sites (TES), promoters, CDS, introns, 5′ UTR, 3′ UTR and intergenic regions were downloaded from UCSC genome browser. The enrichment of transcriptional factor motifs in peaks was evaluated using HOMER (http://homer.ucsd.edu/homer/motif/). For downstream analysis, we normalised the read counts by computing counts scaled by the number of sequenced fragments multiplied by one million (CPM).

### Whole Genome Bisulfite Sequencing (WGBS) Analysis

WGBS libraries were prepared using the TruSeq DNA Methylation Library Preparation Kit (Illumina). Briefly, genomic DNA was isolated using the DNeasy Blood & Tissue Kit (Qiagen) according to the manufacturer's guide. Then, approximately 500 ng DNA were bisulfite treated using EZ DNA Methylation Kit (Zymo Research). Bisulfite-converted DNA was end-repaired, dA-tailed, and ligated with adapters following instructions of the TruSeq DNA Methylation Library Preparation Kit. Finally, high quality indexed libraries were then pooled and sequenced on Illumina NovoSeq platform with 150-bp paired-end reads.

WGBS data analysis was followed our established analysis pipelines [58, 59]. Briefly, WGBS raw data were removed first 12-bp at the 5′ end of both pairs, and reads with adapters and low-quality bases by using TrimGalore-0.4.3. The trimmed sequences were mapped to the bovine genome (ARS-UCD1.2) using Bismark. Uniquely mapped reads were then removed PCR duplicated reads and non-converted reads using deduplicate_bismark and filter_non_conversion. For avoiding the sequencing bias, only reads with 10× coverage was used in the downstream analysis. Methylation of each CpG site was calculated and methylation DNA methylation of each sample was calculated by averaging the consecutive genomic window of 300-bp tiles' methylation. Genomic features, including promoters (1000 bp upstream of transcription start site), exons, introns, CpG islands, intergenic regions were downloaded from University of California, Santa Cruz (UCSC) genome browser. The gene ontology and pathway analysis were performed by means of the David tool [56].

### REFERENCES CITED IN THIS EXAMPLE

1. Wooding, F. B. and D. C. Wathes, Binucleate cell migration in the bovine placentome. J Reprod Fertil, 1980. 59(2): p. 425-30.
2. Wooding, F. B., Current topic: the synepitheliochorial placenta of ruminants: binucleate cell fusions and hormone production. Placenta, 1992. 13(2): p. 101-13.
3. Haeger, J.-D., N. Hambruch, and C. Pfarrer, The bovine placenta in vivo and in vitro. Theriogenology, 2016. 86(1): p. 306-312.
4. Sigdel, A., R. S. Bisinotto, and F. Peñagaricano, Genes and pathways associated with pregnancy loss in dairy cattle. Sci Rep, 2021. 11(1): p. 13329.
5. Gao, G., et al., Transcriptome-wide analysis of the SCNT bovine abnormal placenta during mid to late gestation. Sci Rep, 2019. 9(1): p. 20035.
6. Davies, C. J., et al., Major histocompatibility antigen expression on the bovine placenta: its relationship to abnormal pregnancies and retained placenta. Anim Reprod Sci, 2004. 82-83: p. 267-80.
7. Tanaka, S., et al., Promotion of trophoblast stem cell proliferation by FGF4. Science, 1998. 282(5396): p. 2072-5.
8. Okae, H., et al., Derivation of Human Trophoblast Stem Cells. Cell Stem Cell, 2018. 22(1): p. 50-63.e6.
9. Matsumoto, S., et al., Establishment of macaque trophoblast stem cell lines derived from cynomolgus monkey blastocysts. Sci Rep, 2020. 10(1): p. 6827.
10. Talbot, N. C., et al., Bovine blastocyst-derived trophectoderm and endoderm cell cultures: interferon tau and transferrin expression as respective in vitro markers. Biol Reprod, 2000. 62(2): p. 235-47.
11. Shimada, A., et al., Isolation and characterization of a bovine blastocyst-derived trophoblastic cell line, BT-1: development of a culture system in the absence of feeder cell. Placenta, 2001. 22(7): p. 652-62.
12. Pillai, V. V., et al., Physiological profile of undifferentiated bovine blastocyst-derived trophoblasts. Biol Open, 2019. 8(5).
13. Yang, Y., et al., Derivation of Pluripotent Stem Cells with In Vivo Embryonic and Extraembryonic Potency. Cell, 2017. 169(2): p. 243-257 e25.
14. Io, S., et al., Capturing human trophoblast development with naive pluripotent stem cells in vitro. Cell Stem Cell, 2021. 28(6): p. 1023-1039 e13.
15. Guo, G., et al., Human naive epiblast cells possess unrestricted lineage potential. Cell Stem Cell, 2021. 28(6): p. 1040-1056 e6.
16. Posfai, E., et al., Evaluating totipotency using criteria of increasing stringency. Nat Cell Biol, 2021. 23(1): p. 49-60.
17. Yang, Y., et al., Derivation of Pluripotent Stem Cells with In Vivo Embryonic and Extraembryonic Potency. Cell, 2017. 169(2): p. 243-257.e25.
18. Gao, X., et al., Establishment of porcine and human expanded potential stem cells. Nat Cell Biol, 2019. 21(6): p. 687-699.
19. Costa, C. B., et al., Influence of cAMP modulator supplementation of in vitro culture medium on Bos taurus indicus embryos. Theriogenology, 2020. 141: p. 134-141.
20. Msheik, H., et al., Transcriptomic profiling of trophoblast fusion using BeWo and JEG-3 cell lines. Mol Hum Reprod, 2019. 25(12): p. 811-824.
21. Mamo, S., et al., RNA sequencing reveals novel gene clusters in bovine conceptuses associated with maternal recognition of pregnancy and implantation. Biology of reproduction, 2011. 85(6): p. 1143-1151.
22. Nakaya, Y., et al., The characterization of DNA methylation-mediated regulation of bovine placental lactogen and bovine prolactin-related protein-1 genes. BMC Molecular Biology, 2009. 10(1): p. 1-14.
23. Bazer, F. W., G. Wu, and G. A. Johnson, Pregnancy recognition signals in mammals: the roles of interferons and estrogens. Animal Reproduction (AR), 2018. 14(1): p. 7-29.
24. McCoski, S. R., et al., Validation of an interferon stimulatory response element reporter gene assay for quantifying type I interferons. Domest Anim Endocrinol, 2014. 47: p. 22-6.
25. Koshi, K., et al., Bovine trophoblastic cell differentiation and binucleation involves enhanced endogenous retrovirus element expression. Reprod Biol Endocrinol, 2012. 10: p. 41.
26. Mamo, S., et al., RNA sequencing reveals novel gene clusters in bovine conceptuses associated with maternal recognition of pregnancy and implantation. Biol Reprod, 2011. 85(6): p. 1143-51.
27. Polei, M., et al., Trophoblast cell differentiation in the bovine placenta: differentially expressed genes between uninucleate trophoblast cells and trophoblast giant cells are involved in the composition and remodeling of the extracellular matrix and O-glycan biosynthesis. BMC Mol Cell Biol, 2020. 21(1): p. 1.
28. Brooks, K., G. Burns, and T. E. Spencer, Conceptus elongation in ruminants: roles of progesterone, prostaglandin, interferon tau and cortisol. J Anim Sci Biotechnol, 2014. 5(1): p. 53.
29. Luo, N., et al., Screening Candidate Genes Regulating Placental Development from Trophoblast Transcriptome at Early Pregnancy in Dazu Black Goats (Capra hircus). Animals (Basel), 2021. 11(7).
30. You, Y., et al., TNF-α Regulated Endometrial Stroma Secretome Promotes Trophoblast Invasion. Front Immunol, 2021. 12: p. 737401.
31. Li, W., et al., Tumor necrosis factor stimulates matrix metalloproteinase 9 secretion from cultured human chorionic trophoblast cells through TNF receptor 1 signaling to IKBKB-NFKB and MAPK1/3 pathway. Biol Reprod, 2010. 83(3): p. 481-7.
32. Kibschull, M., et al., Connexin31-deficient trophoblast stem cells: a model to analyze the role of gap junction communication in mouse placental development. Dev Biol, 2004. 273(1): p. 63-75.
33. Kizaki, K., et al., Gelatinase (MMP-2 and -9) expression profiles during gestation in the bovine endometrium. Reproductive Biology and Endocrinology, 2008. 6(1): p. 1-11.
34. Zhao, L., et al., Establishment of bovine expanded potential stem cells. Proc Natl Acad Sci USA, 2021. 118(15).
35. Bogliotti, Y. S., et al., Efficient derivation of stable primed pluripotent embryonic stem cells from bovine blastocysts. Proc Natl Acad Sci USA, 2018. 115(9): p. 2090-2095.
36. Ali, A., et al., The Role of LIN28-let-7-ARID3B Pathway in Placental Development. Int J Mol Sci, 2020. 21(10).
37. Gerri, C., et al., Initiation of a conserved trophectoderm program in human, cow and mouse embryos. Nature, 2020. 587(7834): p. 443-447.
38. Saeed-Zidane, M., et al., Hyaluronic acid and epidermal growth factor improved the bovine embryo quality by regulating the DNA methylation and expression patterns of the focal adhesion pathway. PLOS One, 2019. 14(10): p. e0223753.
39. Yang, Y., et al., Derivation of pluripotent stem cells with in vivo embryonic and extraembryonic potency. Cell, 2017. 169(2): p. 243-257. e25.
40. Yu, L., et al., Derivation of intermediate pluripotent stem cells amenable to primordial germ cell specification. Cell Stem Cell, 2021. 28(3): p. 550-567. e12.
41. Mikkelsen, T. S., et al., Genome-wide maps of chromatin state in pluripotent and lineage-committed cells. Nature, 2007. 448(7153): p. 553-560.
42. Latos, P. A. and M. Hemberger, From the stem of the placental tree: trophoblast stem cells and their progeny. Development, 2016. 143(20): p. 3650-3660.
43. Goissis, M. D. and J. B. Cibelli, Functional characterization of SOX2 in bovine preimplantation embryos. Biology of Reproduction, 2014. 90(2): p. 30, 1-10.
44. Adachi, K., et al., Context-dependent wiring of Sox2 regulatory networks for self-renewal of embryonic and trophoblast stem cells. Molecular cell, 2013. 52(3): p. 380-392.
45. Ohinata, Y. and T. Tsukiyama, Establishment of trophoblast stem cells under defined culture conditions in mice. PloS one, 2014. 9(9): p. e107308.
46. Hemberger, M., C. W. Hanna, and W. Dean, Mechanisms of early placental development in mouse and humans. Nature Reviews Genetics, 2020. 21(1): p. 27-43.
47.Simintiras, C. A., et al., The biochemistry surrounding bovine conceptus elongation. Biology of Reproduction, 2019. 101(2): p. 328-337.
48. Hassa, P. O. and M. O. Hottiger, The diverse biological roles of mammalian PARPS, a small but powerful family of poly-ADP-ribose polymerases. Frontiers in Bioscience-Landmark, 2008. 13(8): p. 3046-3082.
49. Kankofer, M. and L. Guz, Is poly(ADP-ribose) polymerase involved in bovine placental retention? Domestic animal endocrinology, 2003. 25(1): p. 61-67.
50. Walter, I. and A. Boos, Matrix metalloproteinases (MMP-2 and MMP-9) and tissue inhibitor-2 of matrix metalloproteinases (TIMP-2) in the placenta and interplacental uterine wall in normal cows and in cattle with retention of fetal membranes. Placenta, 2001. 22(5): p. 473-83.
51. Ockenga, W., et al., Non-neuronal functions of the m2 muscarinic acetylcholine receptor. Genes, 2013. 4(2): p. 171-197.
52. Smith, A., Formative pluripotency: the executive phase in a developmental continuum. Development, 2017. 144(3): p. 365-373.
53. Ming, H., et al., The landscape of accessible chromatin in bovine oocytes and early embryos. Epigenetics, 2021. 16(3): p. 300-312.
54. Patro, R., et al., Salmon provides fast and bias-aware quantification of transcript expression. Nat Methods, 2017. 14(4): p. 417-419.
55. Robinson, M. D., D. J. McCarthy, and G. K. Smyth, edgeR: a Bioconductor package for differential expression analysis of digital gene expression data. Bioinformatics, 2010. 26(1): p. 139-40.
56. Huang da, W., B. T. Sherman, and R. A. Lempicki, Bioinformatics enrichment tools: paths toward the comprehensive functional analysis of large gene lists. Nucleic Acids Res, 2009. 37(1): p. 1-13
57. Gutierrez-Castillo, E., et al., Effect of vitrification on global gene expression dynamics of bovine elongating embryos. Reprod Fertil Dev, 2021. 33(5): p. 338-348.
58. Jiang, Z., et al., DNA methylomes of bovine gametes and in vivo produced preimplantation embryos. Biol Reprod, 2018. 99(5): p. 949-959.
59. Duan, J. E., et al., Methylome Dynamics of Bovine Gametes and in vivo Early Embryos. Front Genet, 2019. 10: p. 512.

### Example 6

### Establishment of Bovine Trophoblast Stem Cells

### Summary

Here, we report that a chemical cocktail (LCDM: leukemia inhibitory factor [LIF], CHIR99021, dimethinedene maleate [DiM], minocycline hydrochloride), previously developed for extended pluripotent stem cells (EPSCs) in mice and humans, allows for de novo derivation and long-term culture of bovine trophoblast stem cells (TSCs). Bovine TSCs retain developmental potency to differentiate into mature trophoblast cells and exhibit transcriptomic and epigenetic (chromatin accessibility and DNA methylome) features characteristic of trophectoderm cells from early bovine embryos. The bovine TSCs established in this study will provide a model to study bovine placentation and early pregnancy failure.

### Introduction

Trophoblasts are specialized cells in the placenta that mediate maternal-fetal crosstalk and are originated from the trophectoderm (TE) of the blastocyst. Pregnancy establishment in cattle requires TE elongation, a unique process in ruminants prior to apposition, attachment, and implantation.¹ During this process, undifferentiated TE cells, or so-called trophoblast progenitor cells, will differentiate to mononucleated trophoblast cells and subsequently binucleate giant cells^{2,3} to drive embryo elongation and will eventually fuse with uterine epithelial cells to establish the interface for fetus and mother.⁴

Embryo loss and early pregnancy failure are major causes of infertility in cattle,^{5,6} where the majority of losses occur during the first few weeks of pregnancy.^{7 9} Proper trophoblast development and function are pivotal for the success of pregnancy. However, due to technical and logistic difficulties associated with in vivo experiments in cattle and a lack of manipulatable cell culture models that recapitulate placental cell differentiation in vitro, we still lack a complete understanding of early bovine placental development. To date, trophoblast stem cells (TSCs) have been established from several rodent and primate species including mice,¹⁰ humans,¹¹ and nonhuman primates.¹² Despite several attempts,¹³⁻¹⁵ bona fide bovine TSCs that withstand the rigor of long-term culture have yet to be derived.

In this study, we discovered that the LCDM condition (composed of leukemia inhibitory factor [LIF], CHIR99021, dimethindene maleate [DiM], minocycline hydrochloride [MiH]), which was previously reported for mouse and human extended pluripotent stem cells (EPSCs),¹⁶ supported de novo derivation of TSCs from bovine blastocysts generated through in vitro fertilization (IVF). Bovine TSCs grown in the LCDM condition maintained stable morphology, karyotype, transcriptomic and epigenomic features, and in vitro and in vivo developmental potential after long-term passaging. The bovine TSCs generated and characterized in this study provide an invaluable source of material to study trophoblast development and function in the ruminants in vitro and make possible the assembly of bovine blastocyst-like structures (blastoids).¹⁷

### Results

De Novo Derivation of Bovine TSCs from Blastocysts

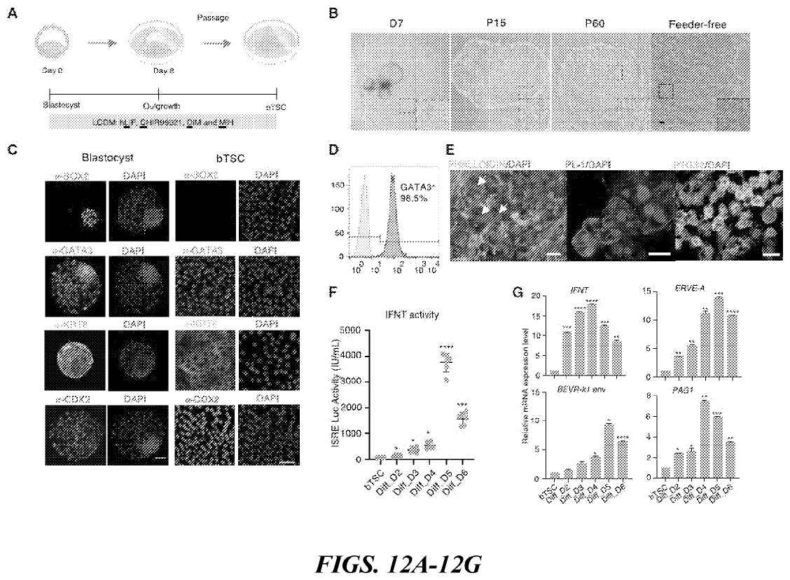

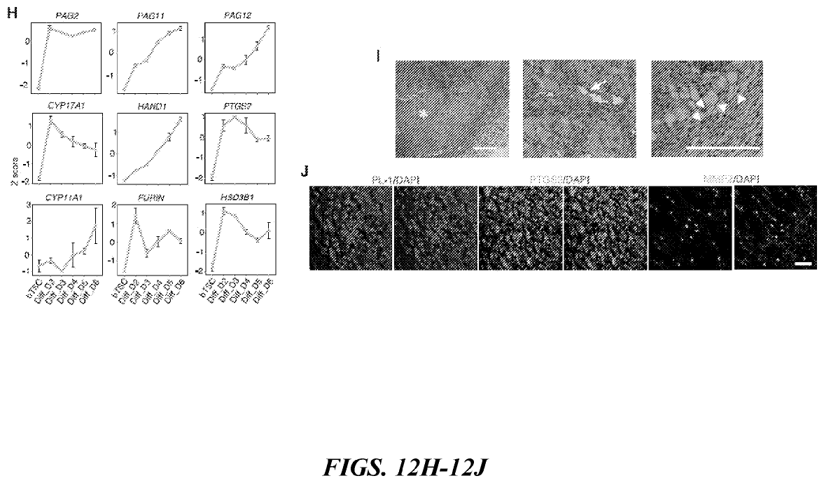

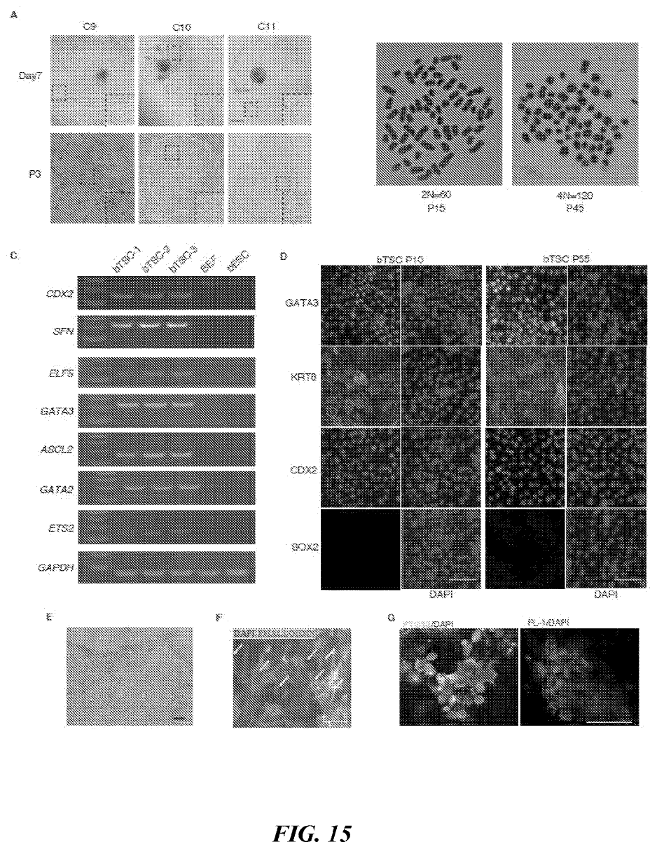

TE cells of bovine blastocysts retain the plasticity to generate inner cell mass (ICM) cells, and vice versa,^{18,19} which prompted us to test de novo derivation of bovine TSCs with different combinations of basal media, growth factors, and chemicals that were previously used for culturing pluripotent stem cells (PSCs) (Table 1). We identified four conditions that can support bovine blastocyst outgrowth for several passages on mouse embryonic fibroblast (MEF) feeder cells (FIG. 12, panel A and panel B; FIG. 15, panel A).

TABLE 1
Culture condition screened for the derivation of bovine TSCs.

| | | | C1 | | C3 | | | | C7 | C8 | | | C11 |
|---|---|---|---|---|---|---|---|---|---|---|---|---|---|
| | | | [S1] | C2 | [S2] | C4 | C5 | C6 | [S3] | [S4] | C9 | C10 | [S5] |
| Basal | N2B27 | | ✓ | | ✓ | ✓ | ✓ | ✓ | | ✓ | ✓ | ✓ | ✓ |
| Mediums | mTeSR | | | ✓ | | | | | | | | | |
| | RPMI1640 | | | | | | | | ✓ | | | | |
| FBS or | FBS (%) | | | | | | | | 20 | | | | |
| KSR | KSR (10%) | | | | | | | | | | 10 | 10 | |
| Factors | TGF-β1 | | ✓ | ✓ | | | | | | | | | |
| | LIF | | ✓ | ✓ | ✓ | ✓ | ✓ | ✓ | | ✓ | ✓ | ✓ | |
| | bFGF | | ✓ | ✓ | | | ✓ | ✓ | | | ✓ | ✓ | ✓ |
| | FGF4 | | | | | | | | ✓ | | | | |
| | Heparin | | | | | | | | ✓ | | | | |
| Small | WNT | CIIIR99021 | ✓ | ✓ | ✓ | ✓ | ✓ | ✓ | | ✓ | ✓ | ✓ | ✓ |
| molecule | MEK1/ | PD0325901 | ✓ | ✓ | | | | | | | ✓ | | |
| | MEK2 | | | | | | | | | | | | |
| | JNK | SP600125 | ✓ | ✓ | | | | ✓ | | | | | |
| | MAPK | SB203580 | ✓ | ✓ | | | | ✓ | | | | | |
| | FGFR1 | SU5402 | | | ✓ | ✓ | ✓ | | | | | | |
| | MEK | PD184352 | | | ✓ | ✓ | ✓ | | | | | | |
| | TGF-β | ActivinA | | | | ✓ | ✓ | | | | ✓ | ✓ | ✓ |
| | muscarinic | DiM | | | | | | | | ✓ | | | |
| | M2/ | | | | | | | | | | | | |
| | histamine | | | | | | | | | | | | |
| | H1 | | | | | | | | | | | | |
| | MMP | MiH | | | | | | | | ✓ | | | |

| | | | | | | | | | | | |
|---|---|---|---|---|---|---|---|---|---|---|---|
| Outgrowth rate (%) | 0 | 0 | 0 | 0 | 20 | 0 | 0 | 89.3 | 58.3 | 66.7 | 53.3 |
| | (0/3) | (0/3) | (0/3) | (0/3) | (1/5) | (0/3) | (0/3) | (25/28) | (7/12) | (8/12) | (8/15) |

Interestingly, an extended pluripotent stem cell (EPSC) culture condition, LCDM (human LIF [hLIF], CHIR99021, DiM, and MiH),¹⁶ was most effective in supporting long-term (>70) passage of bovine TSC-like cells (bTSC-LCs) from blastocyst outgrowth. Removing each hLIF, CHIR99021, DiM, and MiH failed to maintain the morphology and self-renewal of bTSCLCs. bTSC-LCs can also be maintained feeder free on Matrigel in the presence of MEF-conditioned LCDM medium (FIG. 12, panel B).

Further characterization revealed that (1) bTSC-LCs maintained stable colony morphology and a normal diploid number of chromosomes (60) after long-term in vitro culture (FIG. 12, panel B; FIG. 15, panel B); (2) bTSC-LCs highly expressed TE-related transcription factors (TFs) (CDX2, SFN, ELF5, GATA3, ASCL2, GATA2, and ETS2) (FIG. 15, panel C); (3) similar to TE cells in bovine blastocysts, at the protein level, bTSC-LCs expressed CDX2, GATA3, and KRT8 but not SOX2 (FIG. 12, panel C; FIG. 15, panel D); and (4) the majority of bTSC-LCs were found GATA3⁺ (FIG. 12, panel D).

Collectively, we discover that an EPSC culture condition supports de novo derivation and long-term self-renewal of stable bTSC-LCs in vitro. bTSC-LCs express bona fide TE-related TFs at mRNA and protein levels, and hereafter we refer to them as bTSCs.

Directed Differentiation of bTSCs into Functional Trophoblast Cells

We next assessed the differentiation potential of bTSCs in vitro. By using a condition containing forskolin, Y27632, and 4% knockout serum replacement (KSR), we were able to differentiate bTSCs into binucleated cells, which expressed trophoblast markers PTGS2 and placental lactogen 1 (PL-1) (FIG. 12, panel E; FIG. 15, panels E-G). In ruminants, interferon τ (IFNτ) produced by mature trophoblast cells is known as a signal for maternal recognition of pregnancy.²⁰ By using a luciferase-based IFN stimulatory response element (ISRE) assay,²¹ we found that IFNτ production significantly increased during bTSC differentiation and peaked around day 5 (FIG. 12, panel F). qRT-PCR analysis further showed that the expression of IFNτ and mature trophoblast markers (BEVR-k1 env, bEPVE-A,²² and pregnancy associated glycoproteins 1 [PAG1]²³) were significantly upregulated following bTSC differentiation (FIG. 12, panel G).

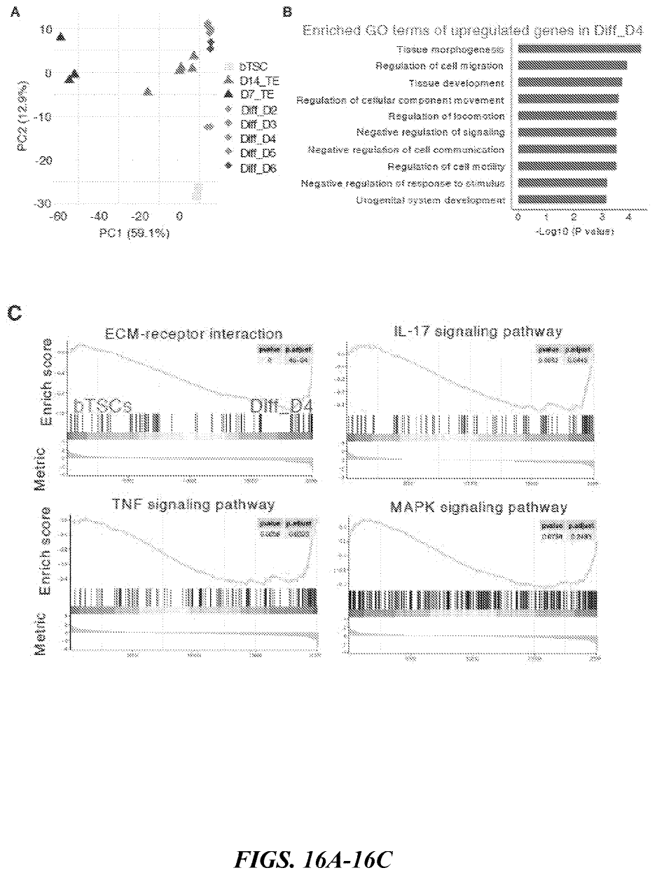

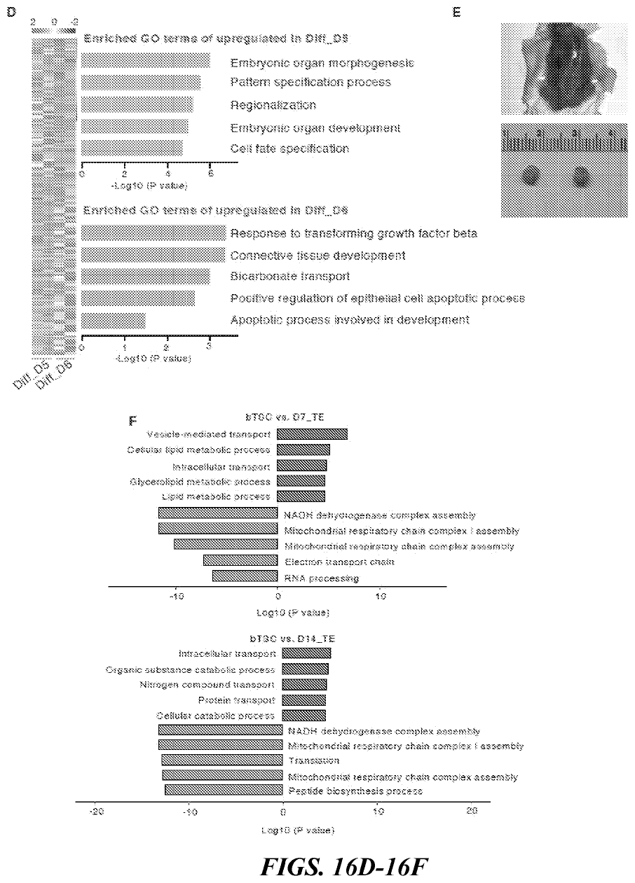

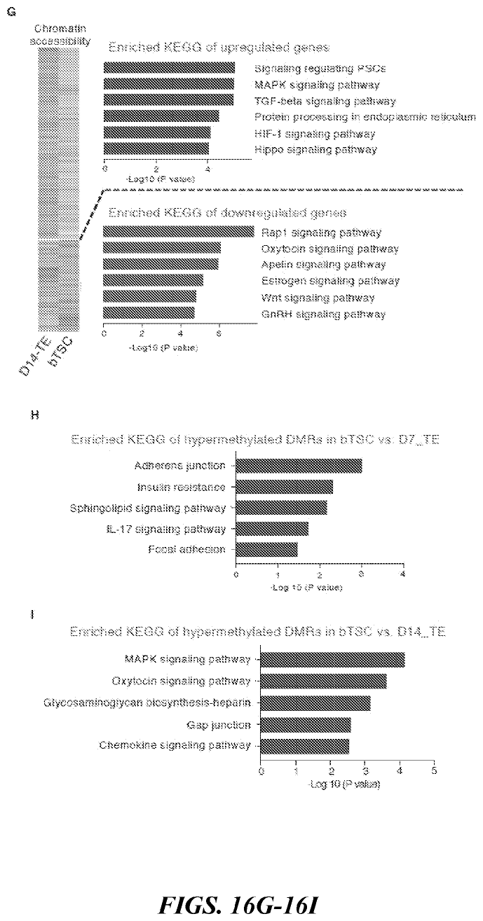

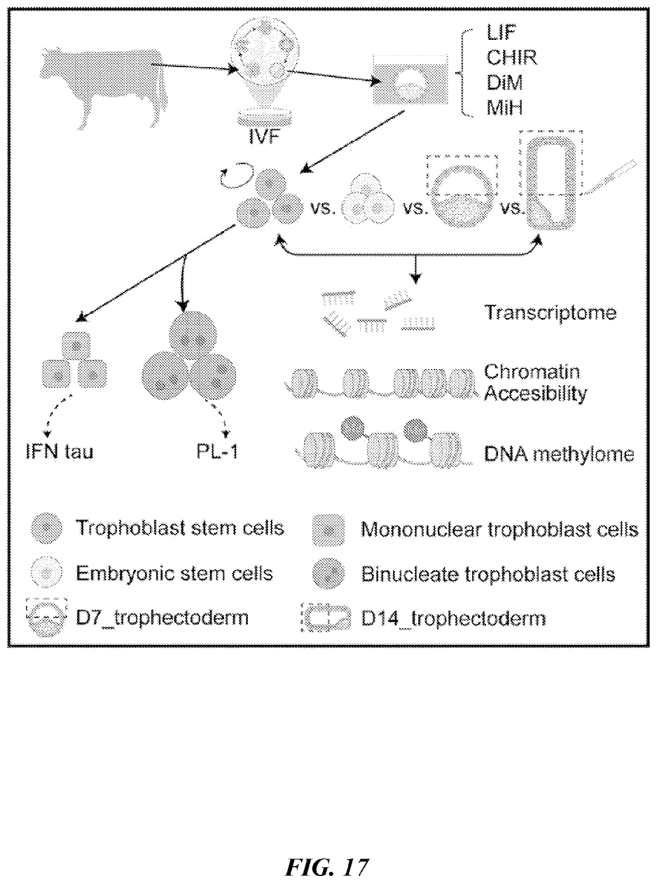

We performed RNA sequencing (RNA-seq) across six time points during bTSC differentiation and found that bTSCs transitioned through an intermediate stage on day 2 before further differentiation into more mature trophoblast cells between days 3 and 6 (FIG. 16, panel A). RNA-seq analysis showed that PAG family genes (PAG2, PAG11, and PAG12) and well known bovine placental marker genes (CYP11A1, CYP17A1, FURIN, HAND1, PTGS2, and HSD3B1^{) 23-25} were upregulated during differentiation (FIG. 12, panel H). Differentiated trophoblasts (day 4) had an upregulation of genes enriched in Gene Ontology (GO) terms related to morphogenesis, cell migration, and locomotion (FIG. 16, panel B), indicating the presence of invasive trophoblast cells. In addition, differentiated trophoblast cells expressed a number of genes involved in extracellular matrix (ECM)-receptor interaction, tumor necrosis factor (TNF), interleukin-17 (IL-17), and MAPK signaling pathways (FIG. 16, panel C), which is consistent with the increase of these signaling activities during implantation and placental development in ruminants and humans.²⁶⁻²⁸ Of note, top GO terms enriched in upregulated genes in day 6 versus day 5 during bTSC differentiation were related to cell apoptosis (FIG. 16, panel D), indicating reduced cell viability. These coincided with the drop of IFNT activity (FIG. 12, panel F) and downregulation of mature trophoblast marker gene expression (FIG. 12, panel G).

We also determined the differentiation potential of bTSCs by subcutaneously injecting them into NOD-SCID mice. By day 9, the injected bTSCs formed ~0.5 cm lesions (FIG. 16, panel E). Immunohistological analysis revealed that the central area of the lesions was necrotic, and the lesions contained blood-filled lacunae-like structures (FIG. 12, panel I), similar to the lesions formed by mouse²⁹ and human TSCs.¹¹ Binucleated cells were identified in the peripheral regions of the lesions and expressed PL-1 and PTGS2, indicating trophoblast maturation (FIG. 12, panel I and panel J). We also identified cells stained positive for MMP2 (a key factor for trophoblast-endometrial epithelia crosstalk and remodeling of endometrial matrices³⁰) located at the peripherals of the lesions (FIG. 12, panel J).

Taken together, these results reveal bTSCs' differentiation potential and demonstrate the ability of bTSCs to generate mature trophoblast cells in vitro and in vivo.

Transcriptional and Epigenomic Features of bTSCs

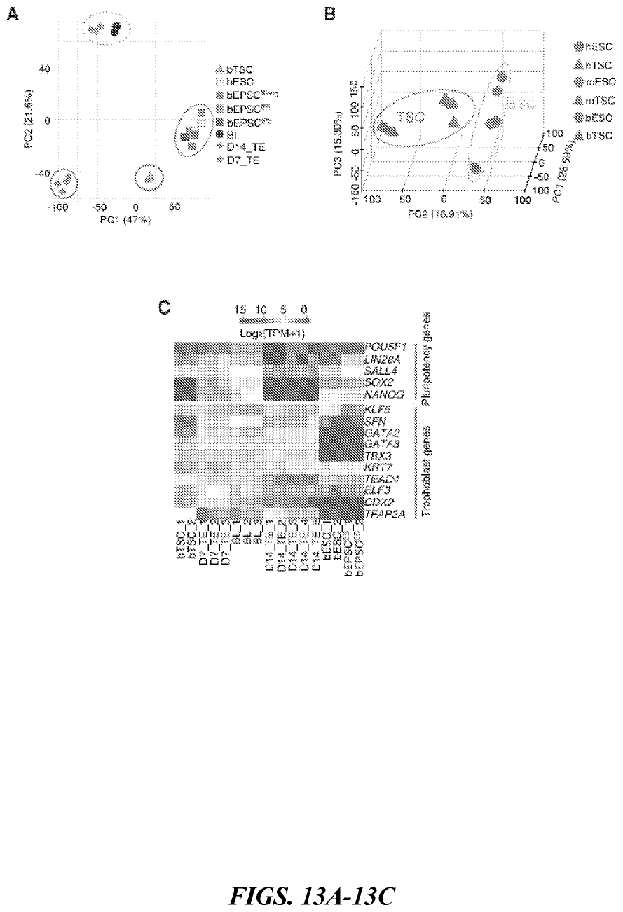

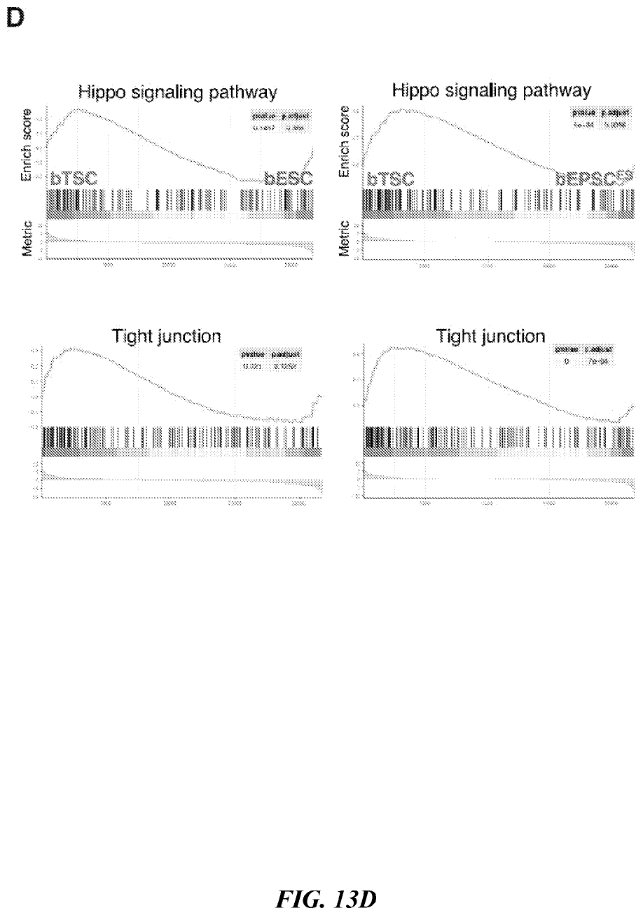

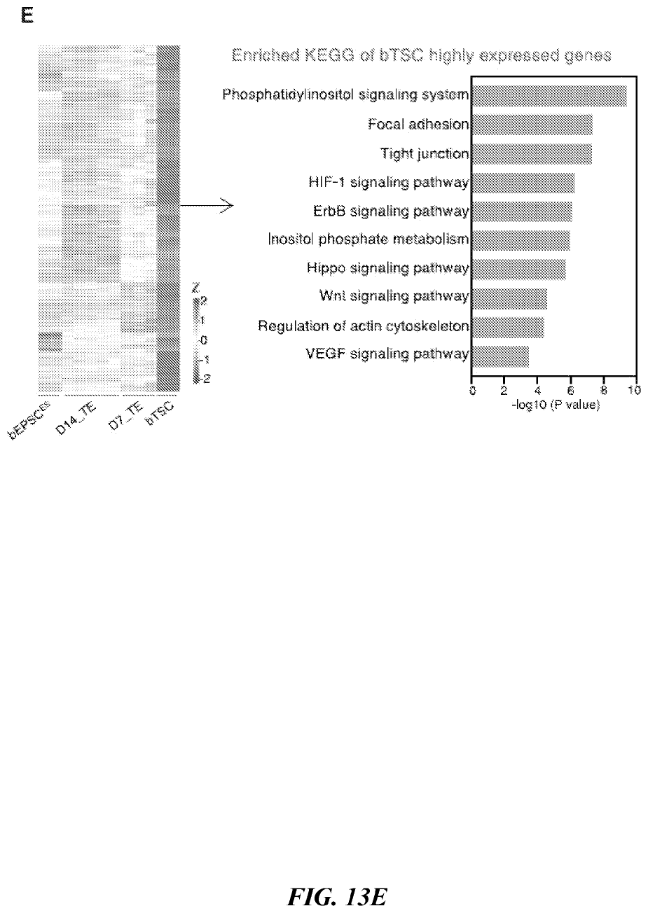

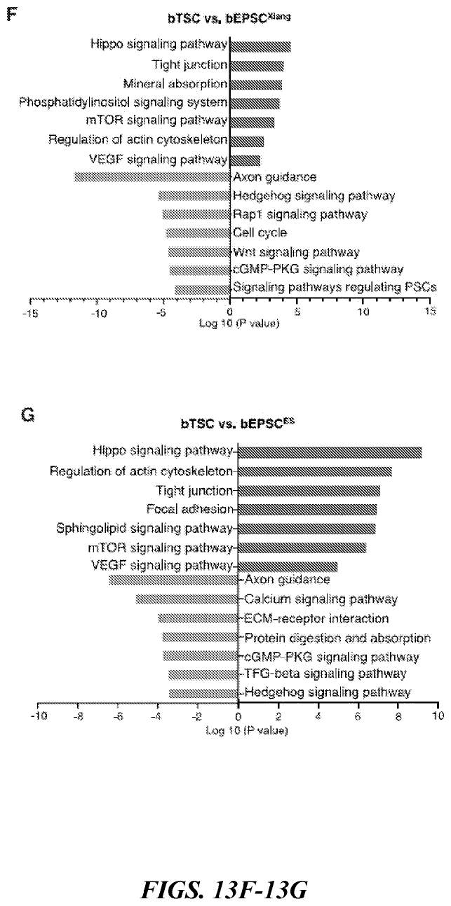

We compared the transcriptomes of bTSCs with those derived from (1) early placental cells at two different developmental stages: TE of preimplantation blastocysts (D7_TE) and day 14 conceptuses (D14_TE),³¹ (2) day 7 IVF blastocysts, and (3) two types of pluripotent stem cells: bovine expanded pluripotent stem cells (bEPSCs: bEPSCs^{ES}, bEPSCs^{iPS,32} and bEPSCs³³) and primed bovine ESCs (bESCs)³⁴ (FIG. 13, panel A). Principal-component analysis revealed that bTSCs were separated from D7_TE, day 7 IVF blastocysts, D14_TE, bESCs, and bEPSCs (FIG. 13, panel A). In addition, bTSCs were distinct from bEPSC³³ that were cultured in the LCDM condition supplemented with KSR serum and higher concentrations of DiM (2 mM), MiH (2 mM), and CHIR99021 (1 mM) (FIG. 13, panel A, panel F, and panel G), while both bEPSCs^{ES} and bEPSCs^{Xiang} that derived from two different conditions showed similar transcriptomic profiles (FIG. 13, panel A).

Additional transcriptomic comparisons of TSCs and ESCs among cattle,³⁴ humans,^{11,35} and mice^{36,37} confirmed the lineage identity of bTSCs (FIG. 13, panel B). bTSCs highly expressed two pluripotency-related genes, LIN28A and SALL4 (FIG. 13, panel C), and trophoblast-related genes KRT7, TEAD3, ELF3, CDX2, and TFAP2A, which is in contrast with TE cells of early embryos (FIG. 13, panel C). In addition, bTSC transcriptomes were enriched with GO terms related to intracellular transport and metabolic process (FIG. 16, panel F) when compared with D7_TE and D14_TE and to Hippo signaling pathway and tight junction when compared with bESCs and bEPSCs^{ES} (FIG. 13, panel D). Of note, signaling pathways including focal adhesion and HIF-1 were also uniquely enriched in bTSCs (FIG. 13, panel E).

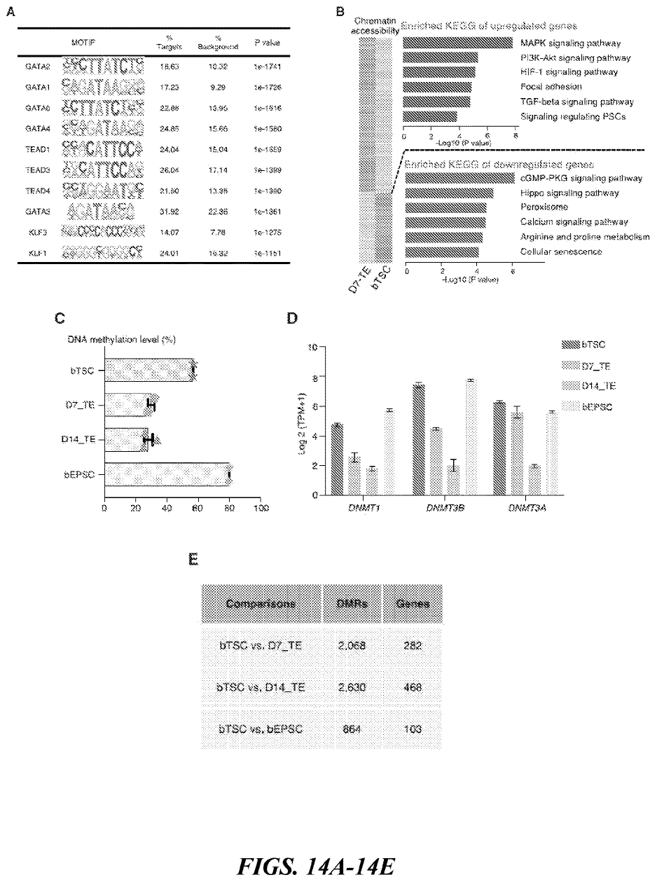

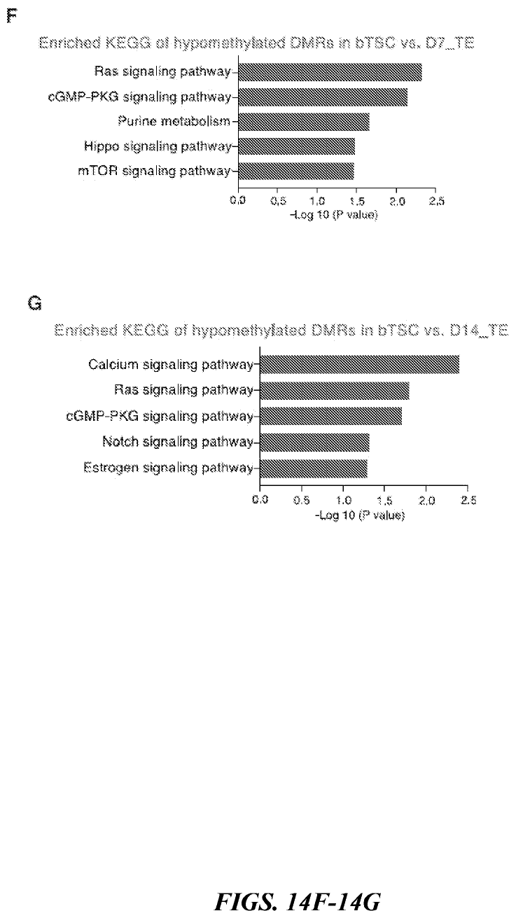

We also performed assay for transposase-accessible chromatin using sequencing (ATAC-seq) and whole-genome bisulfite sequencing (WGBS) analyses and studied epigenomic features of bTSCs. We confirmed that trophoblast TFs were among top enriched binding motifs in bTSCs (FIG. 14, panel A). Analysis of differential enrichment of ATAC-seq peaks between bTSCs and D7_TE/D14_TE further confirmed the overrepresentation of focal adhesion and the HIF-1 signaling pathway in bTSCs (FIG. 14, panel B; FIG. 16, panel G). WGBS analysis showed that the overall methylation level of bTSCs (56.75%) was much higher than those of D7_TE (29.90%) and D14_TE (28.03%) but lower than that of bEPSCs32 (79.80%) (FIG. 14, panel C). This is in line with the higher levels of DNA methyltransferases (DNMT1, DNMT3A, and DNMT3B) in bTSCs and bEPSCs (FIG. 14, panel D). We were able to identify differentially methylated regions (DMRs) between bTSCs and D7_TE/D14_TE (FIG. 14, panel E). Hypomethylated regions in bTSCs compared to D7_TE and D14_TE included genes that were involved in metabolism including Ras, cGMP-PKG, calcium signaling, and purine metabolism (FIG. 14, panel F and panel G). On the contrary, the hypermethylated regions in bTSCs compared with D7_TE were enriched in adherens junction, insulin resistance, sphingolipid, and IL-17 signaling, while MARK, oxytocin, glycosaminoglycan biosynthesis, gap junction, and chemokine signaling were enriched in the hypermethylated regions in bTSCs compared with D14_TE (FIG. 16, panel H and panel I).

Together, our RNA-seq, ATAC-seq, and WGBS analyses provide comprehensive transcriptomic and epigenomic profiles of bTSCs and shed light on the molecular features during the earliest steps of placenta development in bovine.

### Discussion

Here, we demonstrated that an EPSC culture condition (LCDM) 16 can support de novo derivation of stable bTSCs from blastocysts. LCDM-derived bTSCs showed the capacity to self-renew long term in culture while retaining the potential to differentiate into mature trophoblast cells. Comprehensive transcriptome and epigenome analyses of bTSCs and TEs revealed the molecular features during bovine early placenta development and predicted regulators of bovine trophoblast differentiation. As a resource for the community, the data presented here fill a gap and add a reliable stem cell model for research into placenta development of an ungulate species.

The LCDM condition was originally developed for the derivation and long-term culture of mouse and human EPSCs with intra- and interspecies developmental potency, respectively, toward both embryonic and extraembryonic tissues.¹⁶ More recently, the LCDM condition has been successfully used to generate porcine induced pluripotent stem cells (iPSCs)³⁸ from pericytes and embryonic fibroblasts, as well as porcine PSCs (pLCDM) from in vivo blastocysts.³⁹ Interestingly, unlike mouse and human EPSCs, pLCDM is prone to trophoblast differentiation, and TSC-LCs can be generated from pLCDM using the human TSC conditions.¹¹ The LCDM condition was also recently tested in bovine. One study showed that bovine iPSCs can be culture adapted in LCDM medium into EPSCs, as well as directly reprogramed from fetal fibroblasts that exhibit embryonic and extraembryonic potency in bovine-mouse chimeras.³³ Interestingly, however, the LCDM condition failed to support the derivation of EPSCs directly from bovine blastocysts.³³ Consistent with this, under the LCDM condition, we did not find EPSC-like colonies, but TSC-like cells can be readily observed from blastocyst outgrowth. Thus, similar to pigs, it seems that trophoblast lineages are favored in LCDM-cultured bovine blastocysts, which is due to the effect of DiM (inhibitor of muscarinic and histamine H1) and/or MiH (inhibitor of PARP and MMP).³⁹ However, the ability of LCDM to support the generation and long-term culture of bovine iPSCs indicates, with further optimization in future studies, that bovine EPSCs can potentially be derived from blastocysts. The ability to support both TSCs and PSCs demonstrates the “permissive” nature of the LCDM culture condition, which was recently observed in other cultures.⁴⁰⁻⁴² The ability to grow more than one embryonic and extraembryonic stem cell in the same condition will help facilitate the study of lineage crosstalk during early development.

In this study, we found that LIF/STAT3, CHIR99021 (a GSK3 inhibitor that activates the canonical Wnt signaling pathway), DiM, and MiH were indispensable for the derivation and maintenance of bTSCs. It is noted that derivation and culture of human TSCs also requires the activation of Wnt signaling in addition to EGF pathway activation and constitutive inhibition of TGF-β, HDAC, and Rho-associated protein kinase (ROCK) activities.¹¹ In contrast, although transient ROCK inhibition during passaging is beneficial, long-term maintenance of bTSCs does not require constant ROCK inhibition. MiH is known to inhibit PARP, and its family member, PARP1, that plays diverse roles, such as DNA damage, chromatin modification, transcription regulation, and histone modification.⁴³ PARP1 facilitates SOX2 to bind to intractable genomic loci, which drive the expressions of key pluripotency genes.⁴⁴ Inhibition of PARP1 is required for maintenance of extraembryonic developmental potency in mouse EPSCs but did not affect the self-renewal.¹⁶ MiH is also a nonselective inhibition of MMP9 that is expressed in the trophoblast cells. MMP9 can degrade components of the ECM to provide a suitable environment for tissue remodeling and migration of binuclear trophoblast cells.⁴⁵ The supplementation of DiM in porcine IVF embryo culture increased the proportion of trophoblast cells and the total cell number of blastocysts,³⁹ indicating that DiM promotes the differentiation toward the trophoblast lineage. Moreover, MAPK signaling has been reported as one of the important downstream pathways of muscarinic and histamine receptor signaling, which can be inhibited by DiM.⁴⁶ In line with this, we also observed that MAPK signaling-related genes were downregulated in bTSCs. The exact roles of both MiH and DiM in stem cell biology remain elusive and warrant future investigations.

In conclusion, we have established stable bTSCs from IVF blastocysts, which not only can serve as a model to study the unique placentation process in the ruminants and early pregnancy failure but also have allowed for the first generation of blastocyst-like structures (blastoids) from a large livestock species.¹⁷

### REFERENCES CITED IN THIS EXAMPLE

1. Spencer, T. E., Forde, N., and Lonergan, P. (2016). Insights into conceptus elongation and establishment of pregnancy in ruminants. Reprod. Fertil. Dev. 29, 84-100. https://doi.org/10.1071/RD16359.
2. Wooding, F. B., and Wathes, D. C. (1980). Binucleate cell migration in the bovine placentome. J. Reprod. Fertil. 59, 425-430. https://doi.org/10.1530/jrf.0.0590425.
3. Wooding, F. B. (1992). Current topic: the synepitheliochorial placenta of ruminants: binucleate cell fusions and hormone production. Placenta 13, 101-113. https://doi.org/10.1016/0143-4004(92)90025-o.
4. Haeger, J.-D., Hambruch, N., and Pfarrer, C. (2016). The bovine placenta in vivo and in vitro. Theriogenology 86, 306-312.
5. Reese, S. T., Franco, G. A., Poole, R. K., Hood, R., Fernadez Montero, L., Oliveira Filho, R. V., Cooke, R. F., and Pohler, K. G. (2020). Pregnancy loss in beef cattle: a meta-analysis. Anim. Reprod. Sci. 212, 106251. https://doi.org/10.1016/j.anireprosci.2019.106251.
6. Wiltbank, M. C., Baez, G. M., Garcia-Guerra, A., Toledo, M. Z., Monteiro, P. L. J., Melo, L. F., Ochoa, J. C., Santos, J. E. P., and Sartori, R. (2016). Pivotal periods for pregnancy loss during the first trimester of gestation in lactating dairy cows. Theriogenology 86, 239-253. https://doi.org/10.1016/j.theriogenology.2016.04.037.
7. Inskeep, E. K., and Dailey, R. A. (2005). Embryonic death in cattle. Vet. Clin. North Am. Food Anim. Pract. 21, 437-461. https://doi.org/10.1016/j.cvfa.2005.02.002.
8. Boyd, H., Bacsich, P., Young, A., and McCracken, J. A. (1969). Fertilization and embryonic survival in dairy cattle. Br. Vet. J. 125, 87-97. https://doi.org/10.1016/s0007-1935(17)49112-3.
9. Dunne, L. D., Diskin, M. G., and Sreenan, J. M. (2000). Embryo and foetal loss in beef heifers between day 14 of gestation and full term. Anim. Reprod. Sci. 58, 39-44. https://doi.org/10.1016/s0378-4320(99)00088-3.
10. Tanaka, S., Kunath, T., Hadjantonakis, A. K., Nagy, A., and Rossant, J. (1998). Promotion of trophoblast stem cell proliferation by FGF4. Science 282, 2072-2075. https://doi.org/10.1126/science.282.5396.2072.
11. Okae, H., Toh, H., Sato, T., Hiura, H., Takahashi, S., Shirane, K., Kabayama, Y., Suyama, M., Sasaki, H., and Arima, T. (2018). Derivation of human trophoblast stem cells. Cell Stem Cell 22, 50-63.e6. https://doi.org/10.1016/j.stem.2017.11.004.
12. Matsumoto, S., Porter, C. J., Ogasawara, N., Iwatani, C., Tsuchiya, H., Seita, Y., Chang, Y.-W., Okamoto, I., Saitou, M., Ema, M., et al. (2020). Establishment of macaque trophoblast stem cell lines derived from cynomolgus monkey blastocysts. Sci. Rep. 10, 1-15.
13. Talbot, N. C., Caperna, T. J., Edwards, J. L., Garrett, W., Wells, K. D., and Ealy, A. D. (2000). Bovine blastocyst-derived trophectoderm and endoderm cell cultures: interferon tau and transferrin expression as respective in vitro markers. Biol. Reprod. 62, 235-247.
14. Shimada, A., Nakano, H., Takahashi, T., Imai, K., and Hashizume, K. (2001). Isolation and characterization of a bovine blastocyst-derived trophoblastic cell line, BT-1: development of a culture system in the absence of feeder cell. Placenta 22, 652-662. https://doi.org/10.1053/plac.2001.0702.
15. Pillai, V. V., Siqueira, L. G., Das, M., Kei, T. G., Tu, L. N., Herren, A. W., Phinney, B. S., Cheong, S. H., Hansen, P. J., and Selvaraj, V. (2019). Physiological profile of undifferentiated bovine blastocyst-derived trophoblasts. Biol. Open 8, bio037937. https://doi.org/10.1242/bio.037937.
16. Yang, Y., Liu, B., Xu, J., Wang, J., Wu, J., Shi, C., Xu, Y., Dong, J., Wang, C., Lai, W., et al. (2017). Derivation of pluripotent stem cells with in vivo embryonic and extraembryonic potency. Cell 169, 243-257.e25. https://doi.org/10.1016/j.cell.2017.02.005.
17. Pinzo' n-Arteaga, C. A., Wang, Y., Wei, Y., Ribeiro Orsi, A. E., Li, L., Scatolin, G., Liu, L., Sakurai, M., Ye, J., Ming, H., Yu, L., Li, B., Jiang, Z., and Wu, J. (2023). Bovine-blastocyst-like structures derived from stem cell cultures. Cell Stem Cell 30, 611-616.
18. Berg, D. K., Smith, C. S., Pearton, D. J., Wells, D. N., Broadhurst, R., Donnison, M., and Pfeffer, P. L. (2011). Trophectoderm lineage determination in cattle. Dev. Cell 20, 244-255. https://doi.org/10.1016/j.devcel.2011.01.003.
19. Kohri, N., Akizawa, H., Iisaka, S., Bai, H., Yanagawa, Y., Takahashi, M., Komatsu, M., Kawai, M., Nagano, M., and Kawahara, M. (2019). Trophectoderm regeneration to support full-term development in the inner cell mass isolated from bovine blastocyst. J. Biol. Chem. 294, 19209-19223. https://doi.org/10.1074/jbc.RA119.010746.
20. Bazer, F. W., Wu, G., and Johnson, G. A. (2017). Pregnancy recognition signals in mammals: the roles of interferons and estrogens. Anim. Reprod. 14, 7-29.
21. McCoski, S. R., Xie, M., Hall, E. B., Mercadante, P. M., Spencer, T. E., Lonergan, P., and Ealy, A. D. (2014). Validation of an interferon stimulatory response element reporter gene assay for quantifying type I interferons. Domest. Anim. Endocrinol. 47, 22-26. https://doi.org/10.1016/j.domaniend.2013.12.003.
22. Koshi, K., Suzuki, Y., Nakaya, Y., Imai, K., Hosoe, M., Takahashi, T., Kizaki, K., Miyazawa, T., and Hashizume, K. (2012). Bovine trophoblastic cell differentiation and binucleation involves enhanced endogenous retrovirus element expression. Reprod. Biol. Endocrinol. 10, 41. https://doi.org/10.1186/1477-7827-10-41.
23. Mamo, S., Mehta, J. P., McGettigan, P., Fair, T., Spencer, T. E., Bazer, F. W., and Lonergan, P. (2011). RNA sequencing reveals novel gene clusters in bovine conceptuses associated with maternal recognition of pregnancy and implantation. Biol. Reprod. 85, 1143-1151. https://doi.org/10.1095/biolreprod.111.092643.
24. Polei, M., Geunther, J., Koczan, D., and Feurbass, R. (2020). Trophoblast cell differentiation in the bovine placenta: differentially expressed genes between uninucleate trophoblast cells and trophoblast giant cells are involved in the composition and remodeling of the extracellular matrix and O-glycan biosynthesis. BMC Mol. Cell Biol. 21, 1. https://doi.org/10.1186/s12860-020-0246-8.
25. Brooks, K., Burns, G., and Spencer, T. E. (2014). Conceptus elongation in ruminants: roles of progesterone, prostaglandin, interferon tau and cortisol. J. Anim. Sci. Biotechnol. 5, 53. https://doi.org/10.1186/2049-1891-5-53.
26. Luo, N., Cheng, W., Zhou, Y., Gu, B., Zhao, Z., and Zhao, Y. (2021). Screening candidate genes regulating placental development from trophoblast transcriptome at early pregnancy in dazu black goats (Caprahircus). Animals. 11, 2132. https://doi.org/10.3390/ani11072132.
27. You, Y., Stelzl, P., Joseph, D. N., Aldo, P. B., Maxwell, A. J., Dekel, N., Liao, A., Whirledge, S., and Mor, G. (2021). TNF-A regulated endometrial stroma secretome promotes trophoblast invasion. Front. Immunol. 12, 737401. https://doi.org/10.3389/fimmu.2021.737401.
28. Li, W., Li, H., Bocking, A. D., and Challis, J. R. G. (2010). Tumor necrosis factor stimulates matrix metalloproteinase 9 secretion from cultured human chorionic trophoblast cells through TNF receptor 1 signaling to IKBKBNFKB and MAPK1/3 pathway. Biol. Reprod. 83, 481-487. https://doi.org/10.1095/biolreprod. 109.082578.
29. Kibschull, M., Nassiry, M., Dunk, C., Gellhaus, A., Quinn, J. A., Rossant, J., Lye, S. J., and Winterhager, E. (2004). Connexin31-deficient trophoblast stem cells: a model to analyze the role of gap junction communication in mouse placental development. Dev. Biol. 273, 63-75. https://doi.org/10.1016/j.ydbio.2004.04.037.
30. Kizaki, K., Ushizawa, K., Takahashi, T., Yamada, O., Todoroki, J., Sato, T., Ito, A., and Hashizume, K. (2008). Gelatinase (MMP-2 and -9) expression profiles during gestation in the bovine endometrium. Reprod. Biol. Endocrinol. 6. 66-11.
31. Gutierrez-Castillo, E., Ming, H., Foster, B., Gatenby, L., Mak, C. K., Pinto, C., Bondioli, K., and Jiang, Z. (2021). Effect of vitrification on global gene expression dynamics of bovine elongating embryos. Reprod. Fertil. Dev. 33, 338-348.
32. Zhao, L., Gao, X., Zheng, Y., Wang, Z., Zhao, G., Ren, J., Zhang, J., Wu, J., Wu, B., Chen, Y., et al. (2021). Establishment of bovine expanded potential stem cells. Proc. Natl. Acad. Sci. USA 118, e2018505118. https://doi.org/10.1073/pnas.2018505118.
33. Xiang, J., Wang, H., Zhang, Y., Wang, J., Liu, F., Han, X., Lu, Z., Li, C., Li, Z., Gao, Y., et al. (2021). LCDM medium supports the derivation of bovine extended pluripotent stem cells with embryonic and extraembryonic potency in bovine-mouse chimeras from iPSCs and bovine fetal fibroblasts. FEBS J. 288, 4394-4411.
34. Bogliotti, Y. S., Wu, J., Vilarino, M., Okamura, D., Soto, D. A., Zhong, C., Sakurai, M., Sampaio, R. V., Suzuki, K., Izpisua Belmonte, J. C., and Ross, P. J. (2018). Efficient derivation of stable primed pluripotent embryonic stem cells from bovine blastocysts. Proc. Natl. Acad. Sci. USA 115, 2090-2095. https://doi.org/10.1073/pnas.1716161115.
35. Takashima, Y., Guo, G., Loos, R., Nichols, J., Ficz, G., Krueger, F., Oxley, D., Santos, F., Clarke, J., Mansfield, W., et al. (2014). Resetting transcription factor control circuitry toward ground-state pluripotency in human. Cell 158, 1254-1269.
36. Buecker, C., Srinivasan, R., Wu, Z., Calo, E., Acampora, D., Faial, T., Simeone, A., Tan, M., Swigut, T., and Wysocka, J. (2014). Reorganization of enhancer patterns in transition from naive to primed pluripotency. Cell Stem Cell 14, 838-853.
37. Sun, S., Yano, S., Nakanishi, M. O., Hirose, M., Nakabayashi, K., Hata, K., Ogura, A., and Tanaka, S. (2021). Maintenance of mouse trophoblast stem cells in KSR-based medium allows conventional 3D culture. J. Reprod. Dev. 67, 197-205.
38. Xu, J., Yu, L., Guo, J., Xiang, J., Zheng, Z., Gao, D., Shi, B., Hao, H., Jiao, D., Zhong, L., et al. (2019). Generation of pig induced pluripotent stem cells using an extended pluripotent stem cell culture system. Stem Cell Res. Ther. 10, 1-16.
39. Yu, Z., Zhou, S., Liu, M., Chen, L., Zhang, X., Yang, X., Cui, H., Jiang, C., Lv, Y., Yan, T., et al. (2022). Porcine pluripotent stem cells established from LCDM medium with characteristics differ from human and mouse extended pluripotent stem cells. Stem Cell. 40, 751-762.
40. Sathyanarayanan, A., Ing-Simmons, E., Chen, R., Jeong, H. W., Ozguldez, H. O., Fan, R., Duethorn, B., Kim, K. P., Kim, Y. S., Stehling, M., et al. (2022). Early developmental plasticity enables the induction of an intermediate extraembryonic cell state. Sci. Adv. 8, eab19583. https://doi.org/10.1126/sciadv.ab19583.
41. Wei, Y., Zhang, E., Yu, L., Ci, B., Guo, L., Sakurai, M., Takii, S., Liu, J., Schmitz, D. A., Ding, Y., et al. (2023). Dissecting Embryonic and Extra-embryonic Lineage Crosstalk with Stem Cell Co-culture. Preprint at bioRxiv. https://doi.org/10.1101/2023.03.07.531525.
42. Wu, B., Yang, Z., Liu, Y., Li, J., Chen, C., Li, X., and Bao, S. (2023). A chemically defined system supports two distinct types of stem cell from a single blastocyst and their self-assembly to generate blastoid. Cell Prolif.e13396 https://doi.org/10.1111/cpr.13396.
43. Hassa, P. O., and Hottiger, M. O. (2008). The diverse biological roles of mammalian PARPS, a small but powerful family of poly-ADP-ribose polymerases. Front. Biosci. 13, 3046-3082.
44. Liu, Z., and Kraus, W. L. (2017). Catalytic-independent functions of PARP-1 determine Sox2 pioneer activity at intractable genomic loci. Mol. Cell 65, 589-603.e9.
45. Dilly, M., Hambruch, N., Haeger, J. D., and Pfarrer, C. (2010). Epidermal growth factor (EGF) induces motility and upregulates MMP-9 and TIMP-1 in bovine trophoblast cells. Mol. Reprod. Dev. 77, 622-629.
46. Ockenga, W., K€uhne, S., Bocksberger, S., Banning, A., and Tikkanen, R. (2013). Non-neuronal functions of the m2 muscarinic acetylcholine receptor. Genes 4, 171-197.
47. Kim, D., Langmead, B., and Salzberg, S. L. (2015). HISAT: a fast spliced aligner with low memory requirements. Nat. Methods 12, 357-360.
48. Pertea, M., Pertea, G. M., Antonescu, C. M., Chang, T.-C., Mendell, J. T., and Salzberg, S. L. (2015). StringTie enables improved reconstruction of a transcriptome from RNA-seq reads. Nat. Biotechnol. 33, 290-295.
49. Love, M. I., Huber, W., and Anders, S. (2014). Moderated estimation of fold change and dispersion for RNA-seq data with DESeq2. Genome Biol. 15, 1-21.
50. Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. (2005). Gene set enrichment analysis: a knowledge-based approach for interpreting genome-wide expression profiles. Proc. Natl. Acad. Sci. USA 102, 15545-15550.
51. Langmead, B., and Salzberg, S. L. (2012). Fast gapped-read alignment with Bowtie 2. Nat. Methods 9, 357-359.
52. Zhang, Y., Liu, T., Meyer, C. A., Eeckhoute, J., Johnson, D. S., Bernstein, B. E., Nusbaum, C., Myers, R. M., Brown, M., Li, W., and Liu, X. S. (2008). Model-based analysis of ChIP-seq (MACS). Genome Biol. 9, R137. https://doi.org/10.1186/gb-2008-9-9-r137.
53. Robinson, J. T., Thorvaldsdo' ttir, H., Winckler, W., Guttman, M., Lander, E. S., Getz, G., and Mesirov, J. P. (2011). Integrative genomics viewer. Nat. Biotechnol. 29, 24-26.
54. Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol. Cell 38, 576-589.
55. Ming, H., Sun, J., Pasquariello, R., Gatenby, L., Herrick, J. R., Yuan, Y., Pinto, C. R., Bondioli, K. R., Krisher, R. L., and Jiang, Z. (2021). The landscape of accessible chromatin in bovine oocytes and early embryos. Epigenetics 16, 300-312. https://doi.org/10.1080/15592294.2020.1795602.
56. Huang, D. W., Sherman, B. T., and Lempicki, R. A. (2009). Bioinformatics enrichment tools: paths toward the comprehensive functional analysis of large gene lists. Nucleic Acids Res. 37, 1-13.
57. Jiang, Z., Lin, J., Dong, H., Zheng, X., Marjani, S. L., Duan, J., Ouyang, Z., Chen, J., and Tian, X. C. (2018). DNA methylomes of bovine gametes and in vivo produced preimplantation embryos. Biol. Reprod. 99, 949-959.
58. Duan, J. E., Jiang, Z. C., Alqahtani, F., Mandoiu, I., Dong, H., Zheng, X., Marjani, S. L., Chen, J., and Tian, X. C. (2019). Methylome dynamics of bovine gametes and in vivo early embryos. Front. Genet. 10, 512.

KEY RESOURCES TABLE

| | | |
|---|---|---|
| REAGENT or | | |
| RESOURCE | SOURCE | IDENTIFIER |

ANTIBODIES

| | | |
|---|---|---|
| Anti-SOX2 | Biogenex | Cat# an833; RRID: not available |
| Anti-CDX2 | Biogenex | Cat# MU392A; RRID: AB_2650531 |
| Anti-GATA3 | Cell signaling | Cat# D13C9; RRID: AB_10835690 |
| Anti-KRT8 | Origene | Cat# BP5075; RRID: AB_979823 |
| Anti-MMP2 | Cell signaling | Cat# 40994; RRID: AB_2799191 |
| Anti-PL-1 | Santa Cruz | Cat# sc-376436; RRID: AB_11151405 |
| Anti-PTGS2 | Sigma | Cat# SAB2500267; RRID: AB_10603695 |
| Alexa Fluor 488 | Invitrogen | Cat# A21206; RRID: AB_2535792 |
| anti-rabbit antibody | | |
| Alexa Fluor 555 | Invitrogen | Cat# A31570; RRID: AB_2536180 |
| anti-mouse antibody | | |
| Alexa Fluor 633 | Invitrogen | Cat# A21082; RRID: AB 10562400 |
| anti-goat antibody | | |
| Phalloidin-iFluor 488 | Abcam | Cat# ab 176753; RRID: not available |

CHEMICALS, PEPTIDES, AND RECOMBINANT PROTEINS

| | | |
|---|---|---|
| Recombinant human LIF | Peprotech | Cat# 300-05 |
| CHIR99021 | Sigma | Cat# SML1046 |
| Dimethinedene maleate | Tocris | Cat# 1425 |
| Minocycline | Santa cruz | Cat# sc-203339 |
| hydrochloride | | |
| Y-27632 | Tocris | Cat# 1254 |
| Forskolin | Sigma | Cat# F3917 |
| Recombinant human | Millipore | Car# IF007 |
| IFN-α | | |
| DMEM/F12 | Cytiva hyclone | Car# sh30023.02 |
| Neurobasal medium | Gibco | Car# 21103-049 |
| N2 supplement | Gibco | Car# 17502-048 |
| B27 supplement | Gibco | Car# 17504-044 |
| NEAA | Gibco | Car# 11140-050 |
| GlutaMax | Gibco | Car# 35050-061 |
| 2-Mercaptoethanol | Gibco | Car# 21985023 |
| Dispase | Stem cell tech | Car# 07923 |
| Accutase | Gibco | Car# A1110501 |
| Matrigel | Corning | Car# 354234 |
| TrypLE | Gibco | Car# 12605-010 |
| Col IV | Corning | Car# 354233 |

CRITICAL COMMERCIAL ASSAYS

| | | |
|---|---|---|
| One-glow luciferase | Promega Corp | Car# E6120 |
| reagen | | |
| KaryoMax colccmid | Gibco | Car# 15212012 |
| solution | | |
| Feeder removal | Miltenybiotec | Car# 130-095-531 |
| MicroBeads, mouse | | |

DEPOSITED DATA

| | | |
|---|---|---|
| RNAseq datasets | This study | GEO: GSE220923 |
| ATACseq datasets | This study | GEO: GSE220922 |
| WGBS datasets | This study | GEO: GSE220924 |
| D14_TE RNAseq | Gutierrez-Castillo et al.³¹ | GEO: GSE159891 |
| datasets | | |
| Bovine EPSCs RNAseq | Zhao et al. ³² | GEO: GSE129760 |
| dataset | | |
| Bovine EPSCs WGBS | Zhao et al.³² | GEO: GSE129760 |
| dataset | | |
| Bovine ESCs RNAseq | Bogliotti et al.³⁴ | GEO: GSE110040 |
| dataset | | |
| Human TSC RNAscq | Okac ct al.¹¹ | JGA: JGA00000000122 |
| dataset | | |
| Human ESCs RNAseq | Takashima et al.³⁵ | GEO: GSE60945 |
| dataset | | |
| Mouse ESCs RNAseq | Buecker et al.³⁶ | GEO: GSE56138 |
| dataset | | |
| Mouse TSCs RNAseq | Sun et al.³⁷ | GEO: GSE147023 |
| dataset | | |

SOFTWARE AND ALGORITHMS

| | | |
|---|---|---|
| HISAT2 version2.1.0 | Kim et al.⁴⁷ | http://daehwankimlab.github.io/hisat2/ |
| StringTie version 2.1.4 | Pertea et al.⁴⁸ | https://ccb.jhu.edu/software/stringtie/ |
| DESeq2 | Love et al.⁴⁹ | https://bioconductor.org/packages/ |
| | | release/bioc/html/DESeq2.html |
| GSEA | Subramanian et al.⁵⁰ | https://www.gsea- |
| | | msigdb.org/gsea/index.jsp |
| bowtie2 version 2.3.4.3 | Langmead and Salzberg⁵¹ | https://bowtie- |
| | | bio.sourceforge.net/bowtie2/index.shtml |
| MACS2 | Zhang et al.⁵² | https://pypi.org/project/MACS2/ |
| IGV | Robinson ct al.⁵³ | https://software.broadinstitute. |
| | | org/software/igv/ |
| HOMER | Heinz et al.⁵⁴ | http://homer.ucsd.edu/homer/motif/ |

### Experimental Model and Subject Details

### Animal Care and Use

In vivo embryos were collected from non-lactating, 3-year-old cross-breed (Bos taurus x Bos indicus) cows. 6-month-old NOD-SCID mice (both males and females) were used for TSCs lesion assay. Mice were housed in 12-hr light/12-hr dark cycle.

### Method Details

### Bovine IVF Embryo Production

The IVF embryos used in this study were produced as previously described.⁵⁵ Briefly, bovine cumulus-oocyte complexes (COCs) were aspirated from selected follicles of slaughterhouse ovaries. BO-IVM medium (IVF Bioscience) was used for oocyte in vitro maturation. IVF was performed using cryopreserved semen from a Holstein bull with proven fertility. Embryos were then washed and cultured in BO-IVC medium (IVF Bioscience) at 38.5 °C with 6% CO2. Day 7 blastocysts were collected with the zona pellucida removed and were processed for TSC derivation.

### Bovine Day 14 Elongated Embryo Production

Day 14 elongated embryo were collected from cross-breed cows as previously described.⁵⁵ Briefly, superovulation was achieved using five doses of intramuscular injections of FSH beginning five days after insertion of a Controlled Intra-vaginal Drug Release (CIDR) device. Two doses of prostaglandin F2 alpha were given along with the last two FSH treatments, followed by CIDR removal. Standing estrus (Day 0) was seen approximately 48 h post-prostaglandin injection. GnRH was then administered at estrus. Each cow was inseminated 12- and 24-hours post-standing heat. Elongated embryos were collected by routine non-surgical uterine flushing on day 14 (D14).

### Derivation and Culture of Bovine TSCs

Each blastocyst was placed in a separate well of a 12-well plate that was seeded with mitomycin C-treated mouse embryonic fibroblast (MEF) cells. The embryos were cultured in bovine TSC medium containing DMEM: F12 (Gibco) and Neurobasal medium (Gibco) (1:1), 0.5× N2-supplement (Gibco), 0.5× B27-supplement (Gibco), 1× NEAA (Gibco), 1× GlutaMAX (Gibco), 0.1 mM 2-mercaptoethanol (Gibco), 0.1% BSA (MP biomedicals), 10 ng/mL LIF (Peprotech, 300-05), 3 mM CHIR99021 (Sigma, SML1046), 2 mM Dimethindene maleate (DiM) (Tocris, 1425) and 2 mM Minocycline hydrochloride (MiH) (Santa cruz, sc-203339). The cells were cultured at 38.5 °C and 5% CO2. After 48 hours of plating, the unattached embryos were pressed against to the bottom of the plates with needles under microscope. The culture medium was changed daily. At day 7 or 8, outgrowths were dissociated by Dispase (STEMCELL Technologies) for 5-10 mins at 38.5 °C, followed by twice washes with DMEM/F12. bTSC were passaged mechanically under a microscope. For optimal survival rate, 10 mM Rho-associated protein kinase (ROCK) inhibitor Y-27632 (Tocris, 1254) was added to the culture medium for 24 hours.

Once established, bTSCs were passaged every 6 days at a 1:6 split ratio using Accutase (Gibco, A1110501). Each well of bTSCs was dissociated by 1 mL Accutase for 5 mins at 38.5 °C, the same volume of bTSCs medium was used to dilute Accutase for neutralizing the reaction. bTSCs were cryopreserved by ProFreeze Freezing medium (Lonza, 12-769E) according to the manufacturer's instructions.

For feeder free condition, bTSCs cultured on feeder cells were passaged to Matrigel (Corning, 354234)-coated plates using MEFconditioned-bTSC-medium (MEF-bTSC).

### Differentiation of Bovine TSCs

Bovine TSCs were grown to 80-90% confluence in the bTSCs medium and dissociated with TrypLE (Gibco, 12605-010) for 15 min at 38.5 °C. Then, bTSCs were seeded in a 6-well plate which was coated with 2.5 μg/mL Col IV (Corning, 354233) at a density of 1-1.5×10⁵ cells per well and cultured in 2 mL differentiation medium containing DMEM: F12 and Neurobasal medium (1:1), with 0.5× N2-supplement, 0.5× B27-supplement, 1× NEAA, 1× GlutaMAX, 0.1 mM 2-mercaptoethanol, 0.1% BSA, 2.5 μM Y27632, 2 μM Forskolin (Sigma, F3917) and 4% KSR (Invitrogen, 10828028). The medium was changed every two days.

### Immunofluorescence Analysis

Cells or blastocysts were fixed in 4% paraformaldehyde (PFA) for 20 min at room temperature, and then rinsed in wash buffer (0.1% Triton X-100 and 0.1% polyvinyl pyrrolidone in PBS) for three times. Following fixation, cells were permeabilized with 1% Triton X-100 in PBS for 30 min and then rinsed with wash buffer. Cells were then transferred to blocking buffer (0.1% Triton X-100, 1% BSA and 0.1 Mglycine) for 2 hours at room temperature. Subsequently, the cells were incubated with the primary antibodies overnight at 4 °C. The primary antibodies used in this experiment include anti-SOX2 (Biogenex, an833), anti-CDX2 (Biogenex, MU392A; 1:200), anti-GATA3 (Cellsignaling, D13C9; 1:200), and anti-KRT8 (Origene, BP5075; 1:300). For secondary antibody incubation, the cells were incubated with Fluor 488- or 555- or 633-conjugated secondary antibodies 1 hour at room temperature. ProLong Diamond Antifade (DAPI included) was used to stain nuclei. The images were taken with a fluorescence confocal microscope (Leica).

Paraffin sections were deparaffinized and then boiled in sodium citrate buffer (pH 6.0) for 20 min for antigen retrieval. Sections were blocked in 5% goat serum in TBST for 1 hour and incubated with primary antibodies at 4 °C overnight. The primary antibodies used in this experiment including anti-MMP2 (Cellsignaling, 40994; 1:200), anti-PL-1 (Santa Cruz, sc-376436; 1:200) and anti-PTGS2 (Sigma, SAB2500267; 1:100-1:200). Then, the sections were incubated with fluorescence-conjugated secondary antibodies for one hour at room temperature. Nuclei were stained with DAPI (Invitrogen, D1306).

### Quantitative Real-Time PCR

Total RNA was extracted from cells using RNeasy Micro Kit (Qiagen) according to the manufacture's protocol. First-strand cDNA was synthesized using the iScript cDNA Synthesis Kit (BIO-RAD). The qRT-PCR was performed using SYBR Green PCR Master Mix (BIORAD) with specific primers (Table 2). Data were analyzed using the BIO-RAD software provided with the instrument. The relative gene expression values were calculated using the ΔΔCT method and normalized to internal control GAPDH.

TABLE 2
Primer lists.

| Gene Name | Forward (5′-3′) | Reverse (5′-3′) |
|---|---|---|
| GAPDH | GCCATCAATGACCCCTTCAT | TGCCGTGGGTGGAATCA |
| PAG1 | TCCACTTTCCGGCTTACCAA | CCTTTCATTCTCCCAGATCCAT |
| ERVE-A | GGATCTGACGGGAGACACAAA | CACCAATCCGGGAATCTTCA |
| BERV-K1 env | GGAAATCACCGGGATGTCCT | GGAGAGGAGGCGCTTACCTG |
| IFNT | GCCCTGGTGCTGGTCAGCTA | CATCTTAGTCAGCGAGAGTC |
| CDX2 | AAGACAAATACCGGGTCGTG | CTGCGGTTCTGAAACCAAAT |
| SFN | CACCCAGAACCTGACCACTT | GCAGACATGCTTTCCCTCTC |
| ELF5 | CGAACAAGCCTCCAGAGTTC | TCCTTTGTCCCCACATCTTC |
| GATA3 | CCACCTACCCACCATACGTC | CGGTTCTGTCCGTTCATCTT |
| ASCL2 | ACCCAAGGCTAGTGTGCAAG | CGTCGTCATAAAGCCCTCTC |
| GATA2 | CTACAGCAGTGGGCTCTTCC | GTTCTGCCCGTTCATCTTGT |
| ETS2 | TGTGGCCAGCAGTTACAGAG | TGCTCCTTTTTGAAGCCACT |

### IFNT Activity Analysis

IFNT activity was measured by an established IFN stimulatory response element-reporter assay.²¹ Briefly, 5-10×10⁵ Madin-Darby bovine kidney cells (MDBK) that are stably transduced with an ISRE-Luc reporter were plated into a well of 96-well polystyrene plates with opaque walls and optically clear bottoms (Corning) and cultured in MDBK growth medium (high glucose DMEM, 10% FBS and 1% Pen/Strep) at 37 °C for 4 hours. After removal of MDBK growth medium, 50 μL of sample or standard (Recombinant human IFN-α, IFNA: Millipore, IF007) were added. The standard curve was generated by a 1:3 serial dilution of IFNA. Cells were incubated at 37 °C for 16 hours, then 50 μL One-Glow Luciferase reagent (Promega Corp; E6120) were added into each well, with a final volume of 100 μL. After mixture at a shaker platform for 10 minutes, the measurement was performed in a plate reader.

### TSCs Lesion Assay

bTSCs cells were grown to about 80% confluence in the bTSCs medium and dissociated with TrypLE. 5×10⁶ bovine TS cells were resuspended in 200 μL 1:1 of bTSC medium and Matrigel, and subcutaneously injected into 6-month-old non-obese diabetic (NOD)-severe combined immunodeficiency (SCID) mice. Lesions were collected at day 7 and 9, fixed in 4% PFA overnight at 4 °C for analysis.

### Karyotyping Assay

bTSCs were incubated with bTSC medium containing 1 mL KaryoMAX colcemid solution (Gibco, 15212012) at 38.5 °C for 4-5 hours. Cells were then dissociated using 1 mL Trypsin (Gibco, 25200-056) at 38.5 °C and centrifuged at 300×g for 5 min. The cells were resuspended in 1 mL PBS solution and centrifuged at 400×g for 2 min. The supernatant was aspirated and 500 μL 0.56% KCl was added to resuspend the cells. The cells were incubated for 15 min, then centrifuged at 400×g for 2 min. 1 mL cold fresh Carnoy's fixative (3:1 methanol: acetic acid) was added to resuspend the cells, followed by a 10 min incubation on ice. After centrifuge, 200 μL Carnoy's fixative was added to resuspend the cells. Cells were dropped on the clean slides and air dried and soaked in a solution (1:25 of Giemsa stain (Sigma, GS500): deionized water) for 9 min. Slides were rinsed with deionized water and air dried. The images were taken by Leica DM6B at 1000× magnification under oil immersion.

### RNA Sequencing Analysis

Total RNA of bovine TSCs was extracted using RNeasy Micro Kit (Qiagen). Trophectoderm from day 7 blastocyst was isolated by placing embryos in a Petri dish with phosphate-buffered saline and performing microsurgery using a microblade under a microscope. The RNA-seq libraries were generated by using the Smart-seq2 v4 kit with minor modification from manufacturer's instructions. Briefly, mRNA was captured and amplified with the Smart-seq2 v4 kit (Clontech). After AMPure XP beads purification, amplified RNAs were quality checked by using Agilent High Sensitivity D5000 kit (Agilent Technologies). High-quality amplified RNAs were subject to library preparation (Nextera XT DNA Library Preparation Kit; Illumina) and multiplexed by Nextera XT Indexes (Illumina). After purification of library with AMPure XP beads (Beckman Coulter), the concentration of sequencing libraries was determined by using Qubit dsDNA HS Assay Kit (Life Technologies). The size of sequencing libraries was determined by means of High Sensitivity D5000 Assay in at Tapestation 4200 system (Agilent). Pooled indexed libraries were then sequenced on the Illumina NovaSeq platform with 150-bp paired-end reads.

The String Tie⁴⁸ was applied to quantify the gene expression profile from the raw sequencing data, by using the Ensembl bovine genome annotation (ARS-UCD1.2). Transcript per million reads (TPM) was used as the unit of gene expression. The DESeq2⁴⁹ was applied to identify differentially expressed genes. The TMM algorithm implemented in the DESeq2 package was used to perform normalization of the read counts and estimation of the effective library sizes. Differential expression analysis was performed by the likelihood ratio test implemented in the DESeq2 package. The conventional statistical analyses were performed based on the R platform. The “cor.test” function was used to perform Spearman's rank correlation test. Principal component analysis (PCA) on the gene expression profile was performed by using the “dudi.pca” function within the package “ade4”. The heatmaps were plotted by the “heatmap.2” function within the package “gplots”. The gene ontology and pathway analysis were performed by means of the David tool.⁵⁶

In total, we sequenced two replicates of bTSCs, trophoblasts differentiated at day 2, 3, 4, 5 and 6, three replicates of whole blastocysts and day 7 trophectoderm cells selected from the same batch used for bTSCs derivation. The RNA-seq datasets of bovine day 14 trophectoderm,³¹ ESCs³⁴ and EPSCs³² were downloaded from previous publications, respectively.

### ATAC-Seq Analysis

The ATAC-seq libraries from fresh cells were prepared as previously described.⁵⁵ Shortly, cells or embryos were lysed on ice, then incubated with the Tn5 transposase (TDE1, Illumina) and tagmentation buffer. Tagmentated DNA was purified using MinElute Reaction Cleanup Kit (Qiagen). The ATAC-seq libraries were amplified by Illumina TrueSeq primers and multiplexed by index primers. Finally, high quality indexed libraries were then pooled together and sequenced on Illumina NovaSeq platform with 150-bp paired-end reads.

The ATACseq analysis was followed our established analysis pipeline.³⁵ Quality assessed ATAC-seq reads were aligned to the bovine reference genome using Bowtie 2.3 with following options: -very-sensitive -X 2000 -no-mixed -no-discordant. Alignments resulted from PCR duplicates or locations in mitochondria were excluded. Only unique alignments within each sample were retained for subsequent analysis. ATAC-seq peaks were called separately for each sample by MACS2 with following options: -keep-dup all -nolambda -nomodel. The ATAC-seq bigwig files were generated using bamcoverage from deeptools. The ATAC-seq signals were normalized in the Integrative Genome Viewer genome browser. The annotations of genomic features, including transcription start sites, transcription end sites (TES), promoters, CDS, introns, 5′ UTR, 3′ UTR and intergenic regions were downloaded from UCSC genome browser. The enrichment of transcriptional factor motifs in peaks was evaluated using HOMER (http://homer.ucsd.edu/homer/motif/). For downstream analysis, we normalized the read counts by computing counts scaled by the number of sequenced fragments multiplied by one million (CPM).

### Whole Genome Bisulfite Sequencing (WGBS) Analysis

WGBS libraries were prepared using the TruSeq DNA Methylation Library Preparation Kit (Illumina). Briefly, genomic DNA was isolated using the DNeasy Blood & Tissue Kit (Qiagen) according to the manufacturer's guide. Then, approximately 500 ng DNA were bisulfite treated using EZ DNA Methylation Kit (Zymo Research). Bisulfite-converted DNA was end-repaired, dA-tailed, and ligated with adapters following instructions of the TruSeq DNA Methylation Library Preparation Kit. Finally, high quality indexed libraries were then pooled and sequenced on Illumina NovaSeq platform with 150-bp paired-end reads.

WGBS data analysis was followed our established analysis pipelines.^{57,58} Briefly, WGBS raw data were removed first 12-bp at the 5′ end of both pairs, and reads with adapters and low-quality bases by using TrimGalore-0.4.3. The trimmed sequences were mapped to the bovine genome (ARS-UCD1.2) using Bismark. Uniquely mapped reads were then removed PCR duplicated reads and nonconverted reads using deduplicate_bismark and filter_non_conversion. For avoiding the sequencing bias, only reads with 10× coverage was used in the downstream analysis. Methylation of each CpG site was calculated and methylation DNA methylation of each sample was calculated by averaging the consecutive genomic window of 300-bp tiles' methylation. Differentially methylated regions (DMRs) were defined as common 300-bp tiles between two compared groups, which methylation levels ≥75% in one group, while ≤25% in another, and were significantly different by Fisher's exact test (P-value≤0.05, FDR≤0.05). Hyper- and hypo-methylated tiles were those with DNA methylation levels ≥75% and ≤25%, respectively. The gene ontology and pathway analysis were performed by means of the David tool.⁵⁶

### Quantification and Statistical Analysis

Statistical differences between pairs of datasets were analyzed by two-tailed unpaired t-tests. Values of p<0.05 were considered statistically significant. Quantitative data are presented as the mean±SD. Repeated number was indicated as “n” in figure legends.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

We claim:

1. A method of culturing, expanding or growing a population of cells derived from a mammalian blastocyst, the method comprising culturing the cells derived from a mammalian blastocyst for a period of time in a culture medium, wherein the culture medium comprises human leukemia inhibitory factor (hLIF), an inhibitor of glycogen synthase kinase-3 (GSK-3), an antagonist of muscarinic M2 and histamine H1 receptors, an inhibitor of matrix metalloproteinase (MMP), or any combination thereof.

2. The method of claim 1, wherein the cultured cells remain in an undifferentiated state.

3. The method of claim 1, wherein the cultured cells are capable of differentiation.

4. The method of claim 1, wherein the method further comprises placing a mammalian blastocyst in a vessel seeded with fibroblast cells and adding the culture medium, thereby providing a population of cells derived from a mammalian blastocyst.

5. The method of claim 1, wherein the population of cells comprises trophoblast stem cells, trophoblast stem-like cells, or derivatives thereof.

6. The method of claim 1, wherein the mammal is a bovine.

7. The method of claim 2, wherein the fibroblast cells comprise mouse embryonic fibroblast cells.

8. The method of claim 1, wherein the GSK-3 inhibitor comprises CHIR99021.

9. The method of claim 1, wherein the antagonist of muscarinic M2 and histamine H1 receptors comprises dimethindene maleate (DiM).

10. The method of claim 1, wherein the inhibitor of matrix metalloproteinase (MMP) comprises minocycline hydrochloride (MiH).

11. The method of claim 1, wherein the amount of the human leukemia inhibitory factor (hLIF) is about 10 ng/ml.

12. The method of claim 1, wherein the amount of the glycogen synthase kinase-3 inhibitor is about 3 uM.

13. The method of claim 1, wherein the amount of the muscarinic M2 and histamine H1 receptor antagonist is about 2 uM.

14. The method of claim 1, wherein the amount of the matrix metalloproteinase inhibitor is about 2 uM.

15. The method of claim 2, wherein the vessel comprises a dish, a flask, a well, a tube, or a plate.

16. The method of claim 2, wherein the vessel comprises a solid surface or a porous surface.

17. The method of claim 1, wherein the cells are cultured on a surface coated with extracellular matrix or a component of extracellular matrix.

18. The method of claim 15, wherein the extracellular matrix is Matrigel™ or a Matrigel™-like substance.

19. The method of claim 1, wherein the surface is not Matrigel™.

20. The method of claim 1, wherein the trophoblast stem cells are cultured without fibroblast feeder cells.

21. An in vitro cell culture comprising a population of cells derived from a mammalian blastocyst produced by the method of claim 1.

22. The in vitro cell culture of claim 21, wherein the cells comprise trophoblast stem cells, trophoblast stem cell-like cells, or derivatives.

23. The in vitro cell culture of claim 21, wherein the cells comprise undifferentiated cells.

24. The in vitro cell culture of claim 21, wherein the cells are capable of self-renewal.

25. An isolated cell derived from a mammalian blastocyst, wherein the isolated cell expresses at least one marker of pluripotency.

26. The isolated cell of claim 25, wherein the isolated cell comprises a trophoblast stem cell, a trophoblast stem cell-like cell, or a derivative thereof.

27. The isolated cell of claim 25, wherein the at least one marker comprises GATA3, CDX2, ELF3, TFAP2A, KLF5, KRT8, SFN, DNMT1, DNMT3A, PAG2, PAG11, PAG12, CYP17A1, HSD3B1, HAND1, or any combination thereof.

28. The isolated cell of claim 25, wherein the at least one marker comprises a marker of the Wnt signaling pathway, the LIF signaling pathway, the HIF-1 signaling pathway, the AMPK signaling pathway, or any combination thereof.

29. The isolated cell of claim 25, wherein the cell is undifferentiated.

30. The isolated cell of claim 25, wherein the cell is capable of self-renewal.

31. The isolated cell of claim 25, wherein the cell is capable of differentiation into cells of the trophoblast lineage in vitro and in vivo.

32. The isolated cell of claim 25, wherein the mammal is a bovine.

33. A method of evaluating a candidate compound, the method comprising contacting the in vitro cell culture of claim 21 or the isolated cell of claim 25 with an amount of the candidate compound and evaluating a characteristic of the in vitro cell culture or isolated cell.

34. The method of claim 33, wherein the characteristic is cell growth, cell development, differentiation, apoptosis, trophoblast development, trophoblast activity, or any combination thereof.

35. A cell culture comprising a population of bovine embryonic stem cells in a medium, wherein the medium comprises one or more factors selected from the group consisting of a human leukemia inhibitory factor (hLIF), an inhibitor of glycogen synthase kinase-3 (GSK-3), an antagonist of muscarinic M2 and histamine H1 receptors, and an inhibitor of matrix metalloproteinase (MMP), or any combination thereof.

36. The cell culture of claim 35, wherein the medium comprises a human leukemia inhibitory factor (hLIF), an inhibitor of glycogen synthase kinase-3 (GSK-3), an antagonist of muscarinic M2 and histamine H1 receptors, and an inhibitor of matrix metalloproteinase (MMP).

37. The cell culture of claim 35, wherein the amount of the human leukemia inhibitory factor (hLIF) is about 10 ng/ml.

38. The cell culture of claim 35, wherein the amount of the glycogen synthase kinase-3 inhibitor is about 3 μM.

39. The cell culture of claim 35, wherein the amount of the muscarinic M2 and histamine H1 receptor antagonist is about 2 μM.

40. The cell culture of claim 35, wherein the amount of the matrix metalloproteinase inhibitor is about 2 μM.

41. The cell culture of claim 35, wherein the inhibitor of glycogen synthase kinase-3 (GSK-3) is CHIR99021, the antagonist of muscarinic M2 and histamine H1 receptors is dimethindene maleate (DiM), and the inhibitor of matrix metalloproteinase (MMP) is minocycline hydrochloride (MiH).

42. The cell culture of claim 35, wherein the culture is in a microwell plate.

43. The cell culture of claim 35, wherein the cell culture further comprises a population of trophoblast stem cells.

44. The cell culture of claim 43, wherein the population of trophoblast stem cells comprises a population of bovine trophoblast stem cells.
